(19) United States
(12) Patent Application Publication
(10) Pub. No.: US 2003/0178031 A1
(43) Pub. Date: Sep. 25, 2003

(54) Method for cancer pain treatment

(75) Inventors: Stuart L. Du Pen, Bainbridge Island, WA (US); Anna R. Du Pen, Bainbridge Island, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Du Pen, Inc., Bainbridge Island, WA

(21) Appl. No.: 10/268,179

(22) Filed: Oct. 9, 2002

Related U.S. Application Data
(63) Continuation of application No. 09/565,644, filed on May 5, 2000, now abandoned.
(60) Provisional application No. 60/133,044, filed on May 7, 1999.

(51) Int. Cl. A61B 19/00

(52) U.S. Cl. 128/898

(57) ABSTRACT

A patient pain management system and method that includes assessing patient history; determining a drug treatment in response to assessing patient history; and repeatedly reassessing the pain and assessing side-effects and adjusting the drug treatment to minimize patient pain. The system includes pain assessment tools for assessing patient pain and treatment history; treatment choice tools for determining a pain treatment protocol; pain reassessment tools for reassessing patient pain in response to the pain treatment protocol; and side-effect assessment tools for assessing side-effects experienced by the patient to enable a caregiver to continuously reassess patient pain and comfort and adjust treatment to minimize patient pain and discomfort.

### CROSS-REFERENCE TO RELATED APPLICATION

This application claims the benefit of U.S. Provisional Patent Application No. 60/133,044 filed May 7, 1999, where this provisional application is incorporated herein by reference in its entirety.

### STATEMENT OF GOVERNMENT INTEREST

This invention was made with government support under 5 RO1 CA64877-03 awarded by The National Institutes of Health, National Cancer Institute. The government has certain rights in the invention.

### TECHNICAL FIELD

The present invention pertains to the treatment of pain, and, more particularly to a cancer pain management system that implements an algorithmic process for assessment, treatment decisions, and reassessment that is continuous over time.

### BACKGROUND OF THE INVENTION

Numerous organizations, including academic, private sector, and government agencies, have developed guidelines, standards, and “templates” for pain and symptom management in oncology and end-of-life care. Recent medicolegal controversies surrounding physican-assisted suicide have increased calls for practice guidelines for palliative care. The practical aspects of implementation of such guidelines for pain management have not bee addressed, and subsequent outcome studies measuring guideline effects on the practice have been essentially nonexistent.

The Agency for Health Care Policy and Research (AHCPR) has developed and published Guidelines for Cancer Pain Management. These guidelines provide an evidence-based foundation for treatment recommendations, but they do not provide explicit treatment protocols that can be clinically tested.

### SUMMARY OF THE INVENTION

A system for pain management using an algorithm for cancer patient treatment based on a decision-tree model is provided. The basic decision-process template of the cancer pain algorithm is pain assessment, analgesic drug choice decisions, and reassessment in a reiterative-cycle design that anchors around the balance of analgesic efficacy versus toxicity. Assessment incorporates a differential diagnosis of the pain and reinforces the need to use palliative antitumor therapy, including chemotherapy, hormonal therapy, and radiation therapy as appropriate. The analgesic drug decision-making intervention is a longitudinal process. There is no single intervention, but rather the algorithm process of assessment, treatment decisions, and reassessment is continuous over time. The algorithm is designed to be a process intervention used for the full duration of pain treatment rather than a one-time treatment approach. The process is operationalized with a set of tools, starting from the initial assessment. A clinic flow sheet is used to document the intensity of the pain, note the presence of any neuropathic pain character, and note the presence of any pain- or analgesic-related side effects in much the same way that oncology flow sheets document hematologic trends and chemotherapy dose adjustments. A bulleted set of analgesic guiding principles for opioids, nonsteriodal anti-inflammatory drugs, tricyclic antidepressants, and anticonvulsants is available for the oncology clinic staff for reference. Each drug category guideline contains supplemental information on indications for the drug, specific agents that are recommended, and practical drug administration guidelines that include dosage and titration, route-specific information, and special attention to pain crisis intervention.

The algorithm decision tree directs the oncologist and oncology nurse to comprehensive side-effect protocols, equianalgesic conversion charts, and a primer for intractable pain. Side-effect protocols cover constipation, nausea, dry mouth, sedation, myoclonus, and gastrointestinal distress.

A flow sheet for each patient's chart was created to monitor significant pain and symptom indicators against their analgesic therapy. All of these tools were designed with the goal of maximum ease of use in the outpatient oncology setting as well as the inpatient environment.

In accordance with one aspect of the present invention, a patient pain management method is provided that includes assessing patient history, including assessing the patient's pain and analgesic history; determining an analgesic drug choice in response to assessing the patient history; and reassessing patient pain and assessing side effects experienced by the patient after receiving the analgesic drug choice, and adjusting the analgesic drug choice, repeatedly, to minimize patient pain.

In accordance with another aspect of the present invention, a process for pain treatment is provided. The process includes assessing pain and analgesic treatment history; administering a pain treatment regimen in accordance with and in response to the assessment of pain and analgesic treatment history; and continuously reassessing pain and side effects of the pain treatment regimen and adjusting the pain treatment regimen to minimize pain and negative side effects.

In accordance with yet another aspect of the present invention, a system for pain management is provided. This system includes pain assessment tools for assessing patient pain and treatment history; treatment choice tools for determining a pain treatment protocol in response to the assessment of patient pain and treatment history; pain reassessment tools for reassessing patient pain in response to the pain treatment protocol; and side-effect assessment tools for assessing the side-effects of the pain treatment protocol.

### BRIEF DESCRIPTION OF THE DRAWINGS

The following description will be more readily appreciated and better understood when taken in conjunction with the accompanying drawings, wherein:

FIG. 1 is a body template chart;

FIGS. 2A-C are pain pattern charts for continuous pain, continuous pain with episodic pain, and episodic pain, respectively;

FIG. 3 is three pain scales for assessing pain in accordance with one embodiment of the present invention;

FIG. 4 is a sample cancer pain algorithm progress note;

FIG. 5 is a key for interpreting flowcharts;

FIG. 6 is a pain history and physical flowchart;

FIG. 7 is a pain assessment flowchart;

FIG. 8 is a side effects assessment flowchart;

FIG. 9 is a pain reassessment flowchart;

FIG. 10 is a side effects reassessment flowchart;

FIG. 11 is a drug choice decisions for constant pain with or without episodic pain flowchart;

FIG. 12 is an opioids flowchart;

FIG. 13 is a sequential opiode trials flowchart;

FIG. 14 is an OTFC flowchart;

FIG. 15 is an NSAID flowchart;

FIG. 16 is a tricyclic and other analgesic antidepressants flowchart;

FIG. 17 is a co-analgesic drug choice flowchart;

FIG. 18 is an anticonvulsants flowchart;

FIG. 19 is a constipation flowchart;

FIG. 20 is a nausea/vomiting flowchart;

FIG. 21 is an over-sedation flowchart;

FIG. 22 is a dry mouth flowchart;

FIG. 23 is a delirium flowchart;

FIG. 24 is a Myoclonus flowchart;

FIG. 25 is a GI distress flowchart;

FIG. 26 is a paranteral opiods administration flowchart;

FIG. 27 is a regional flowchart;

FIG. 28 is a spinal administration/procedures flowchart;

FIG. 29 is a transdermal opioids administration flowchart.

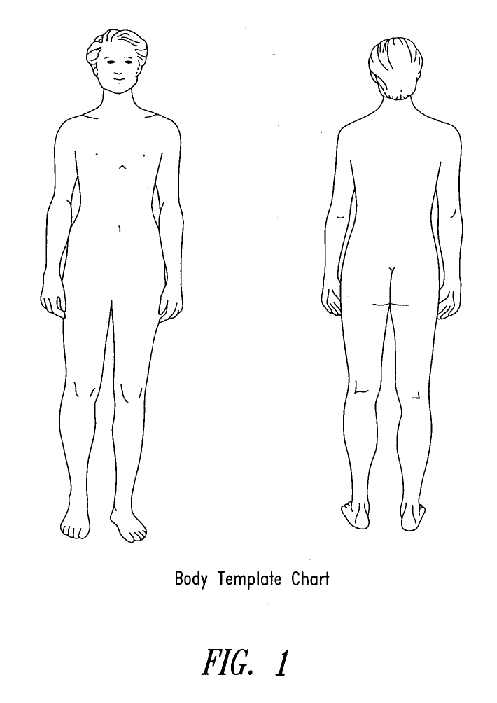

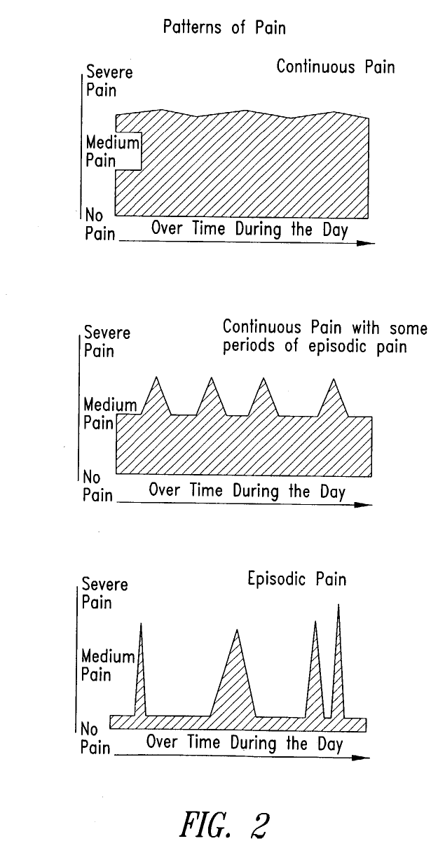

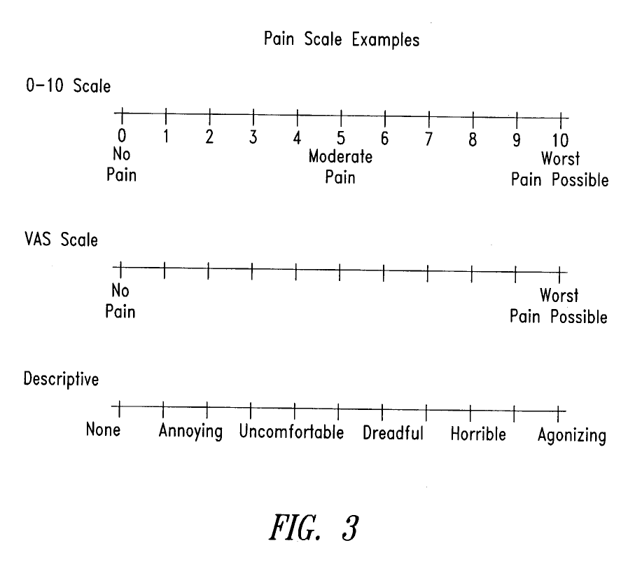

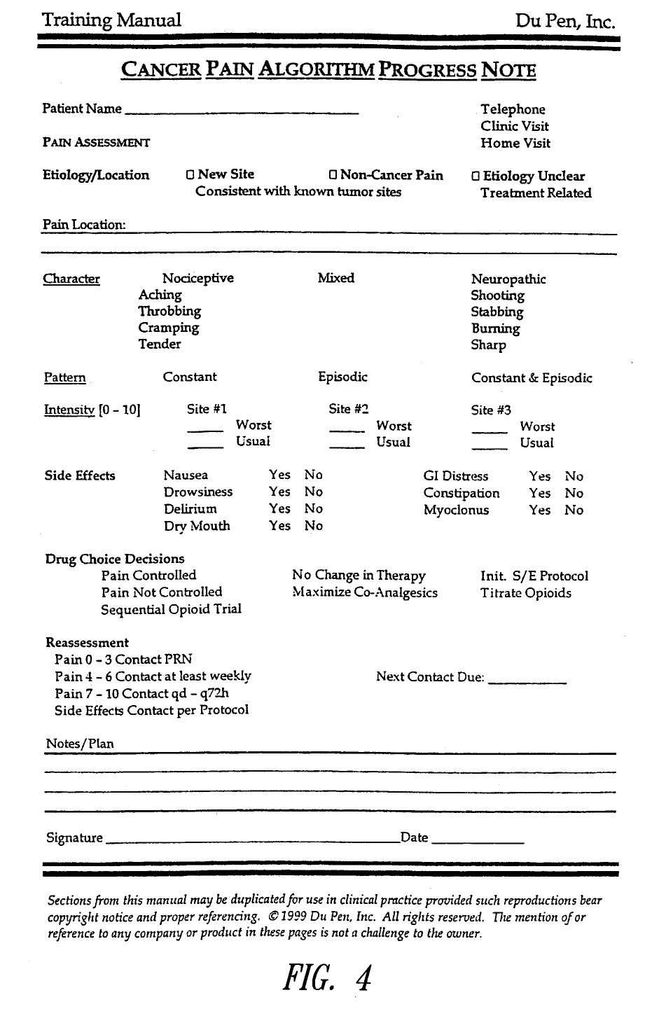

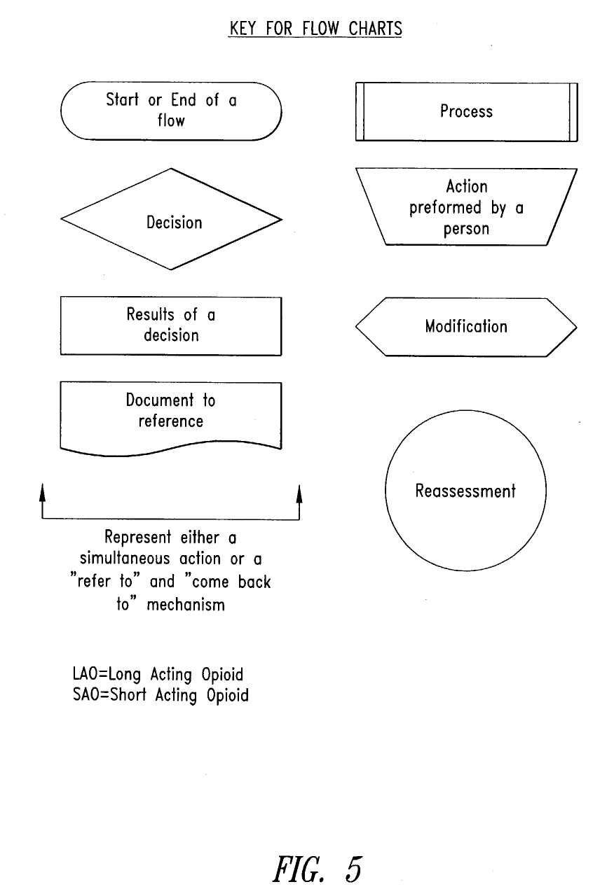

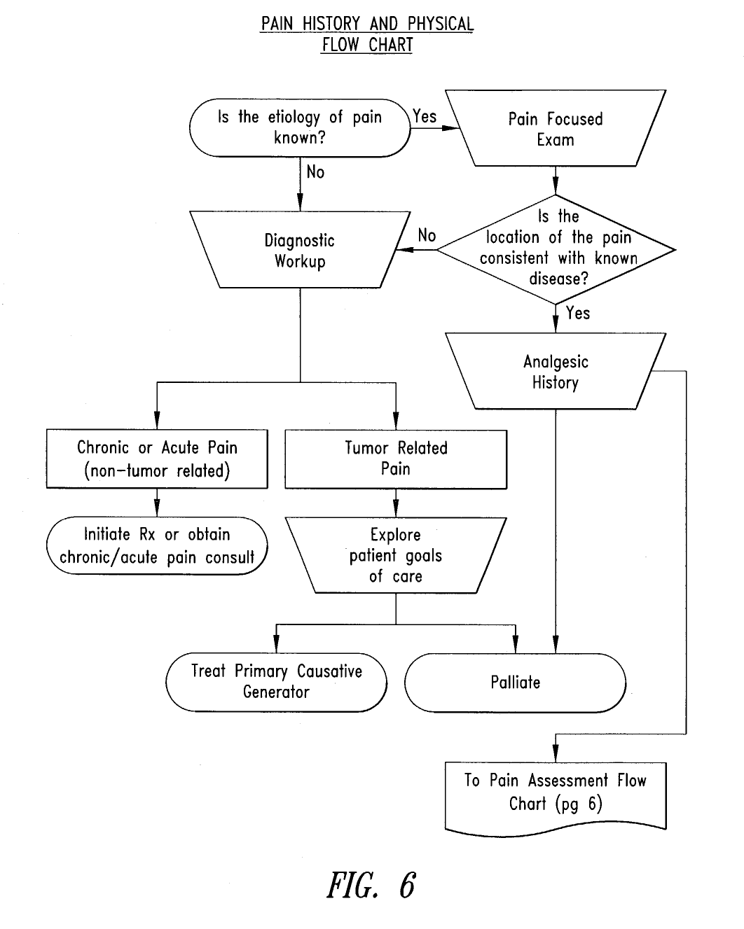

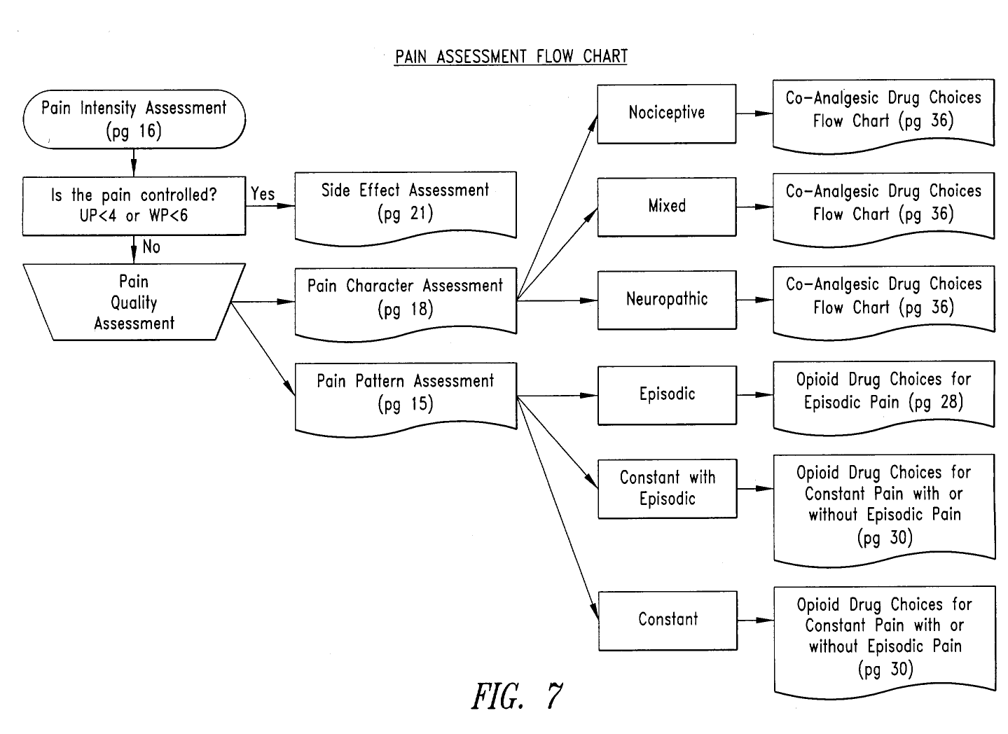

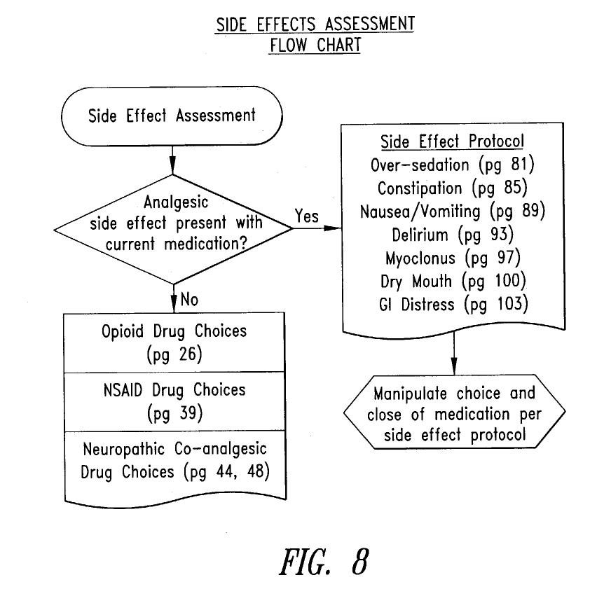

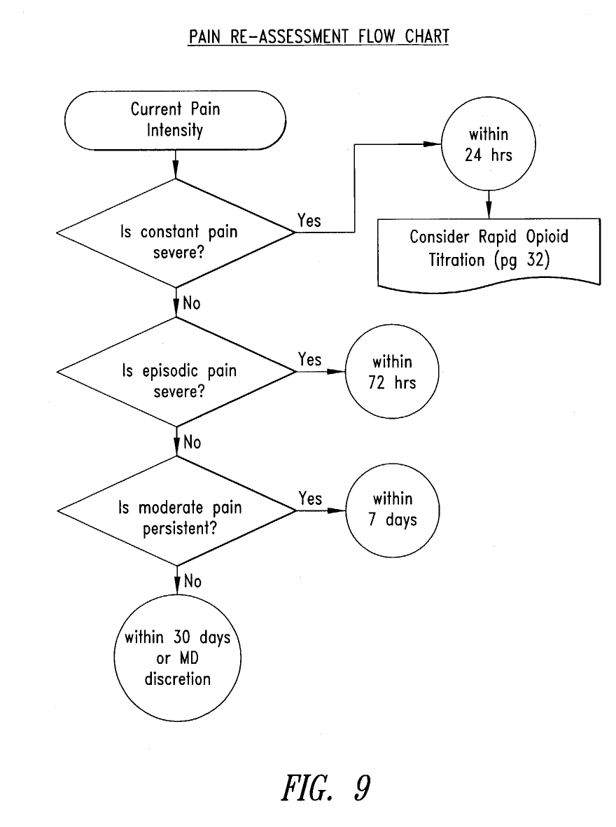

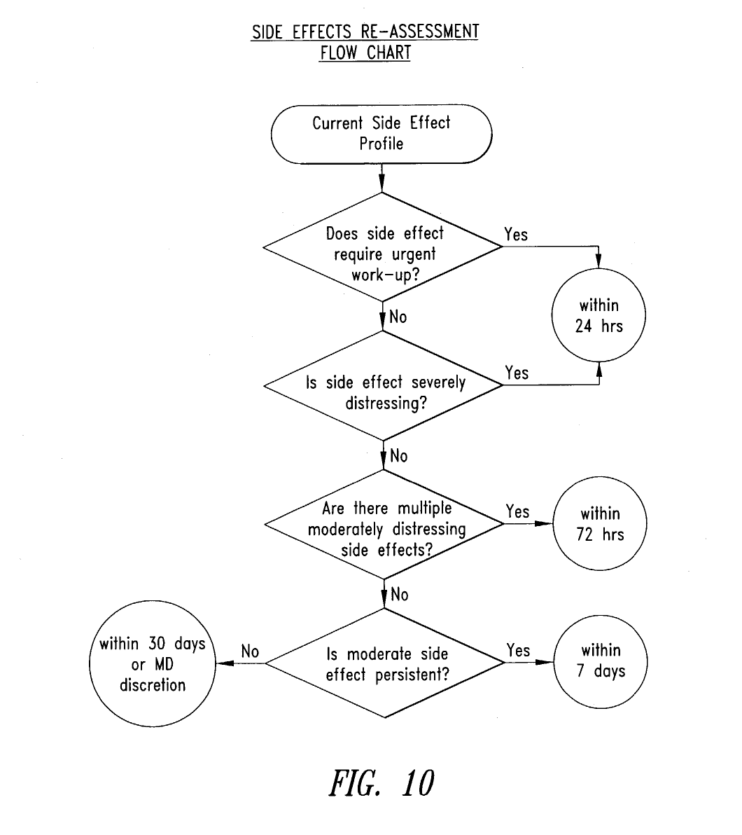

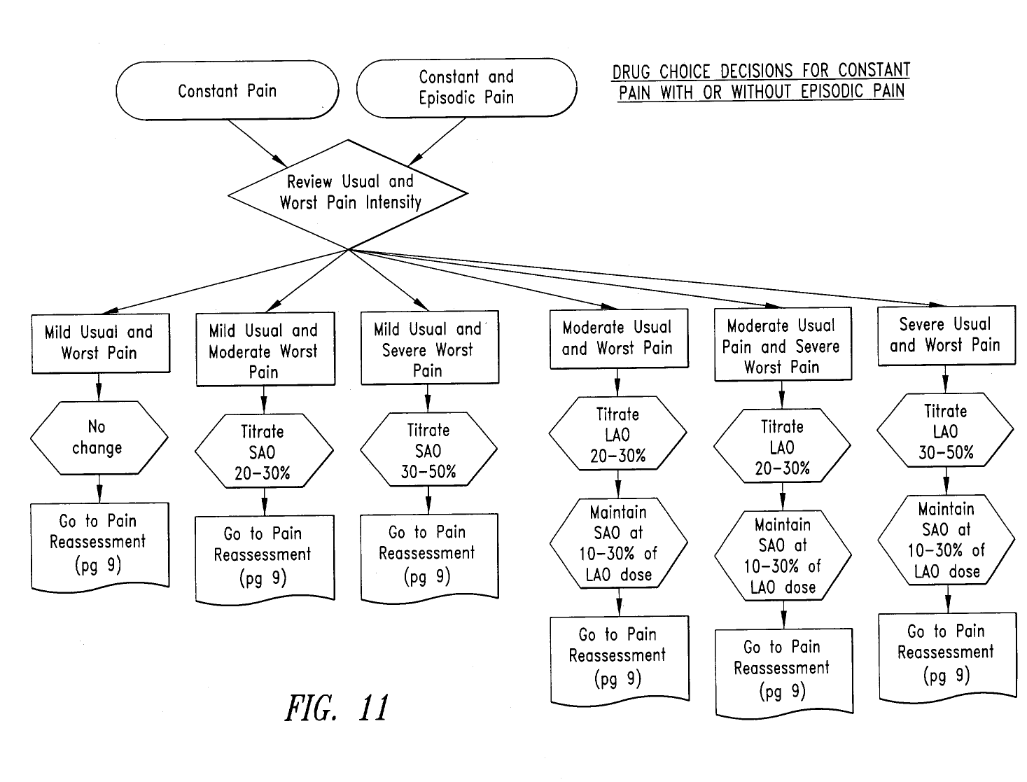

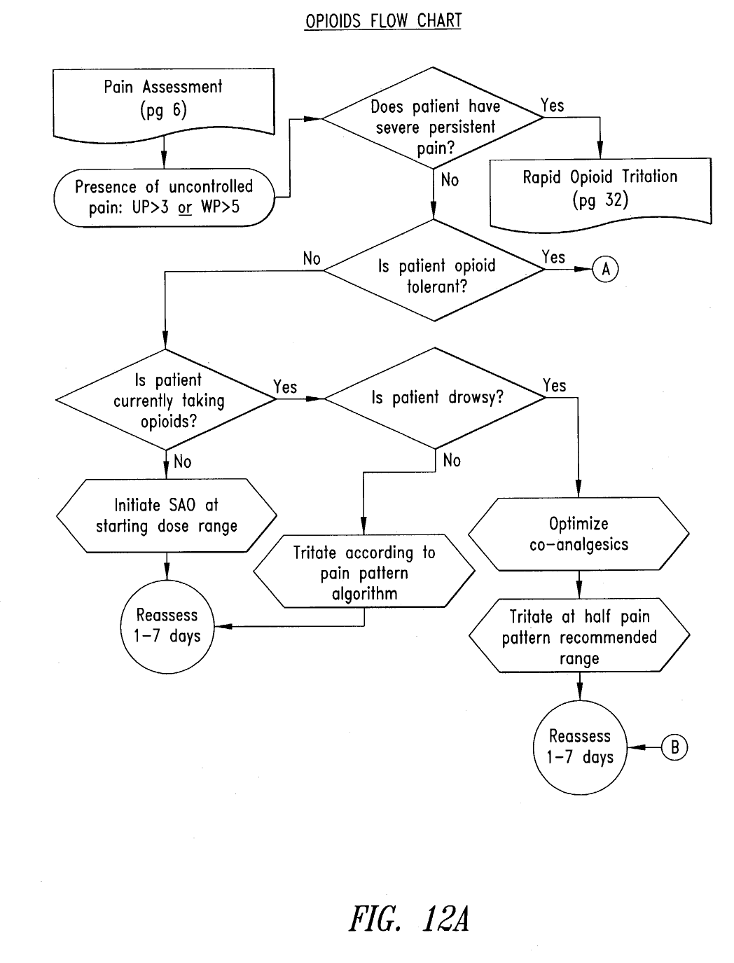

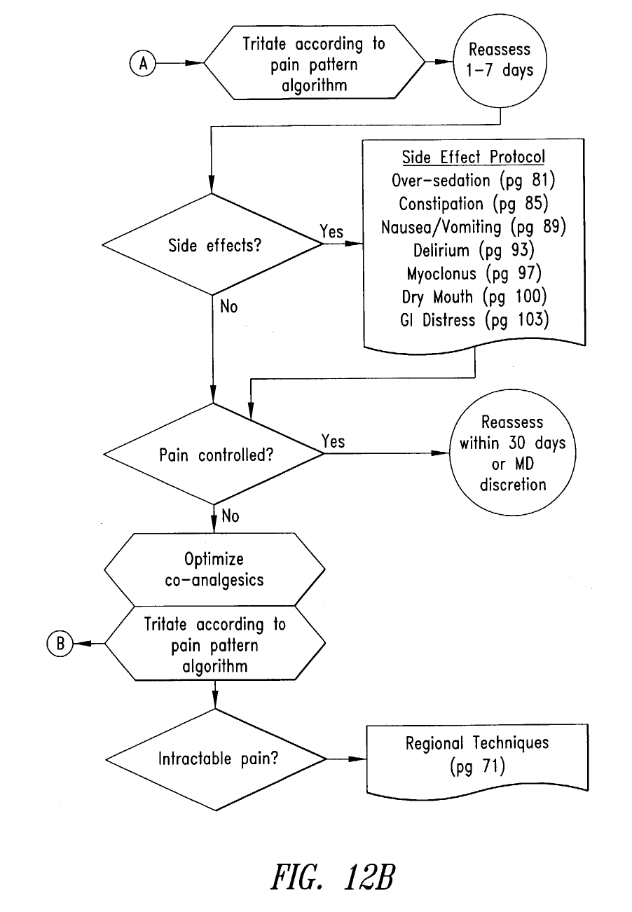

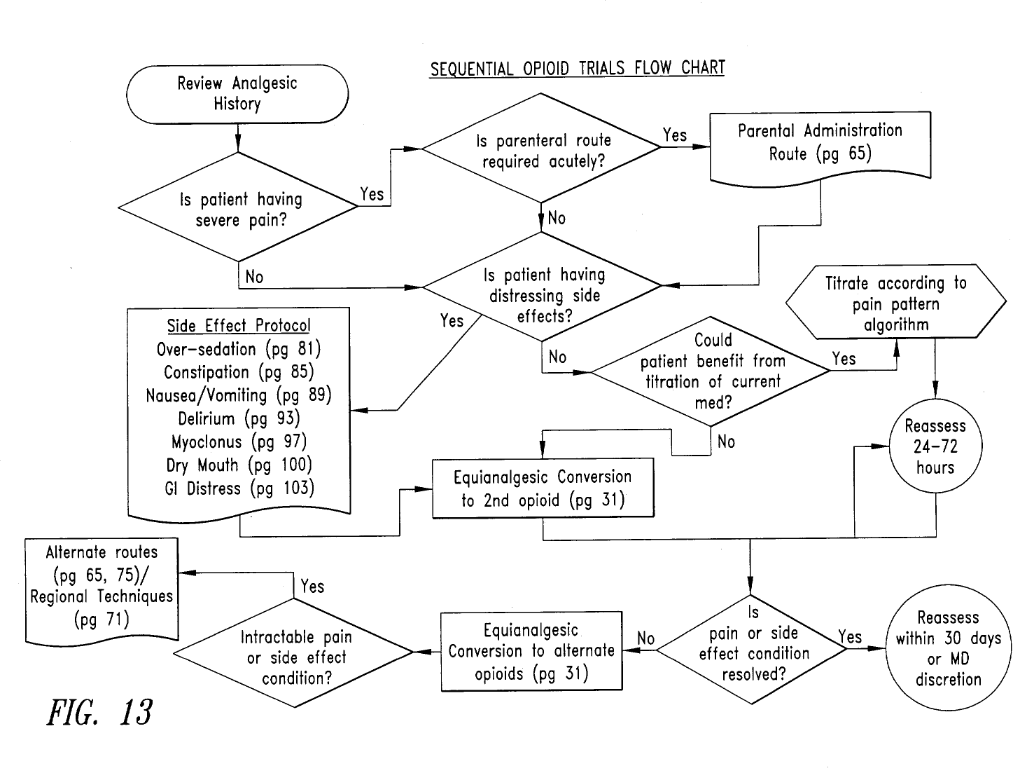

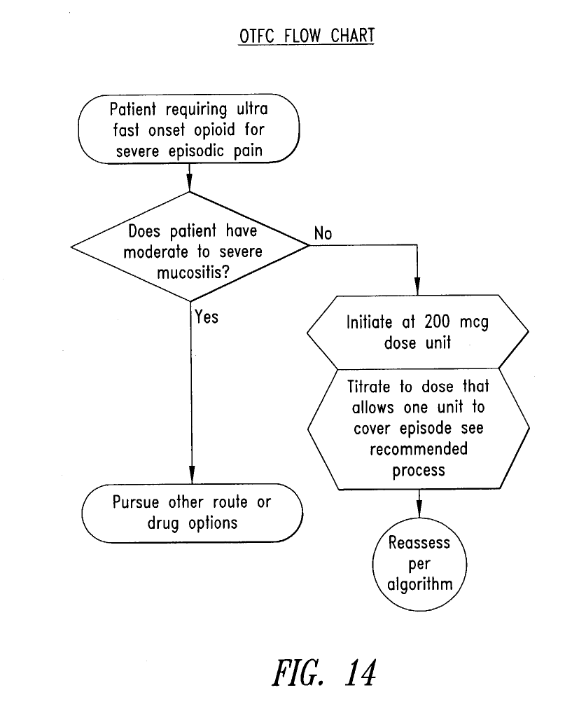

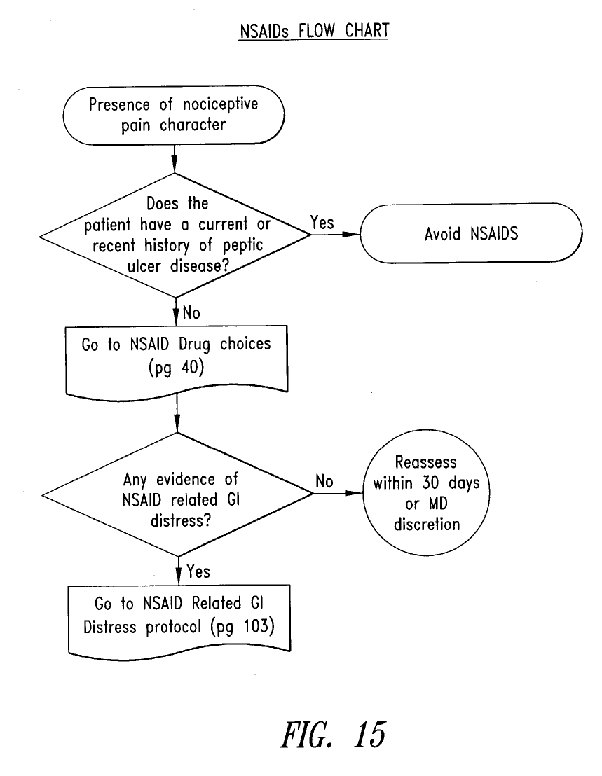

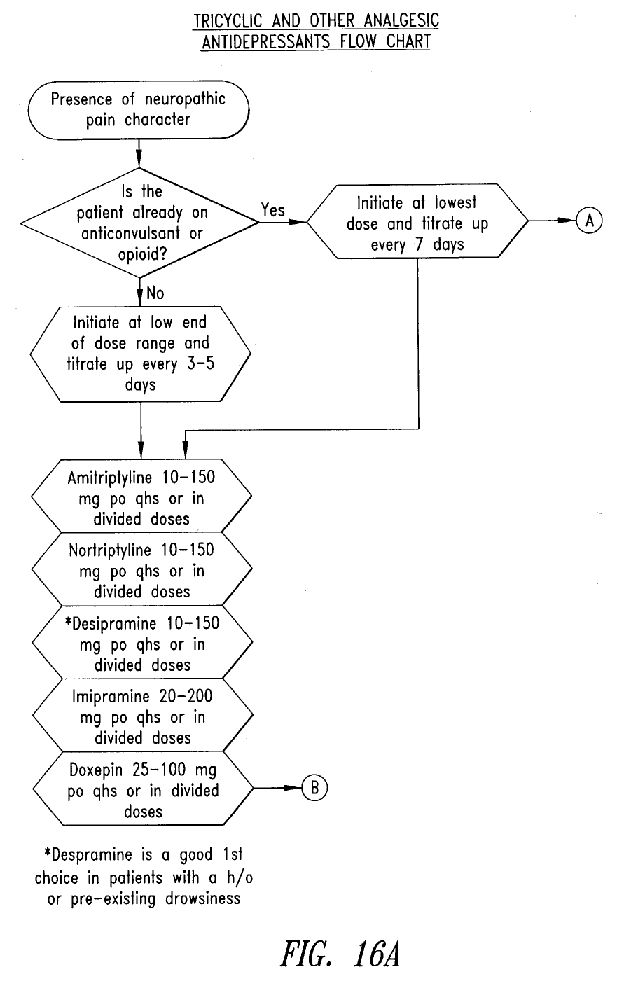

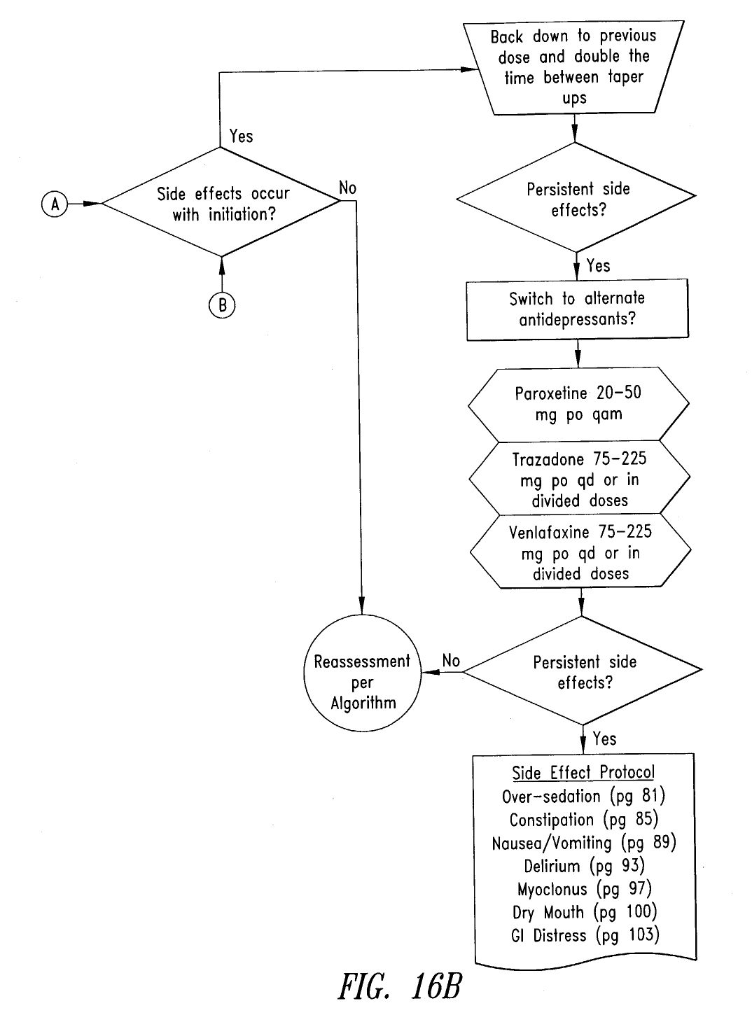

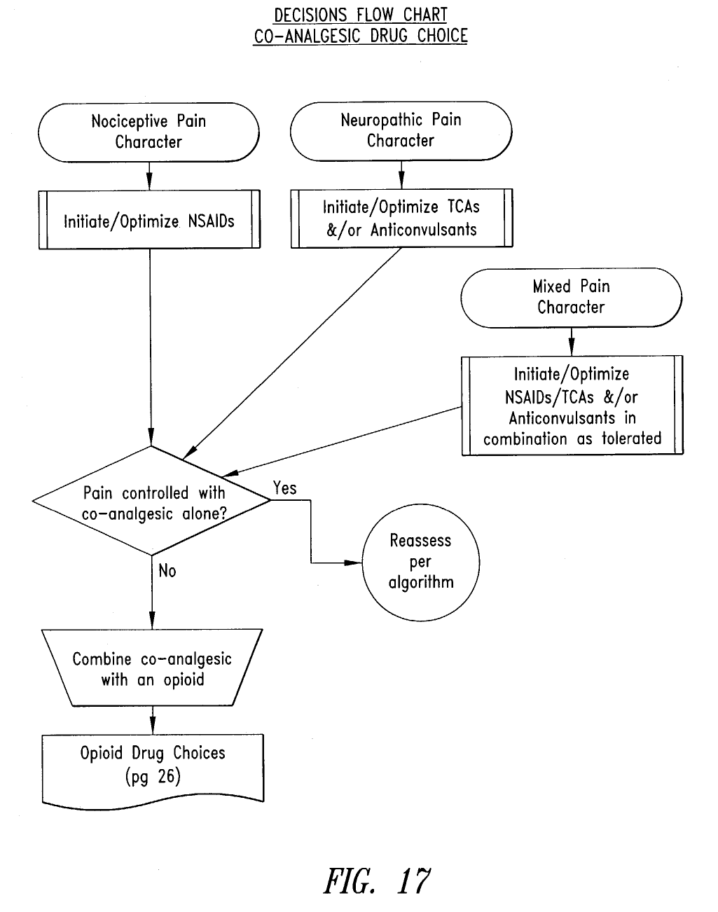

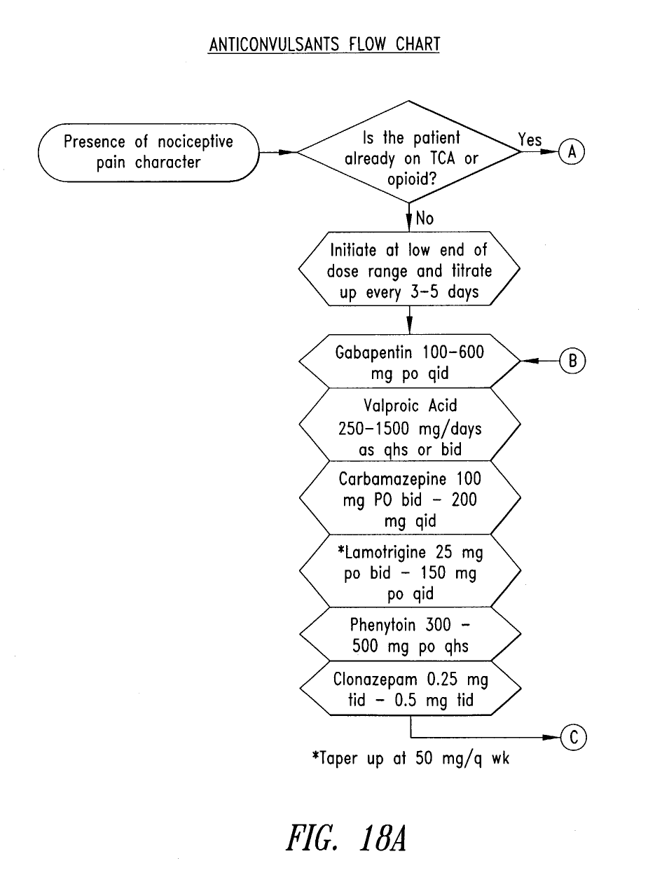

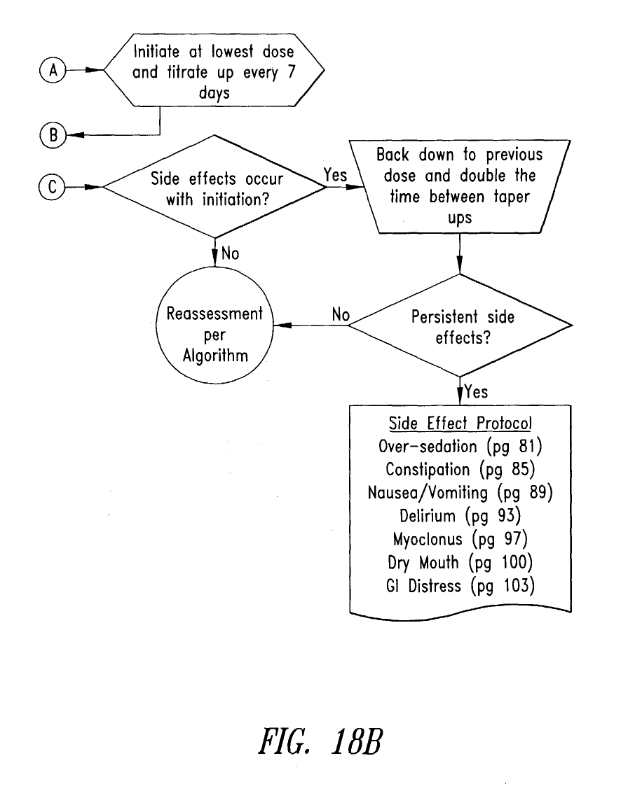

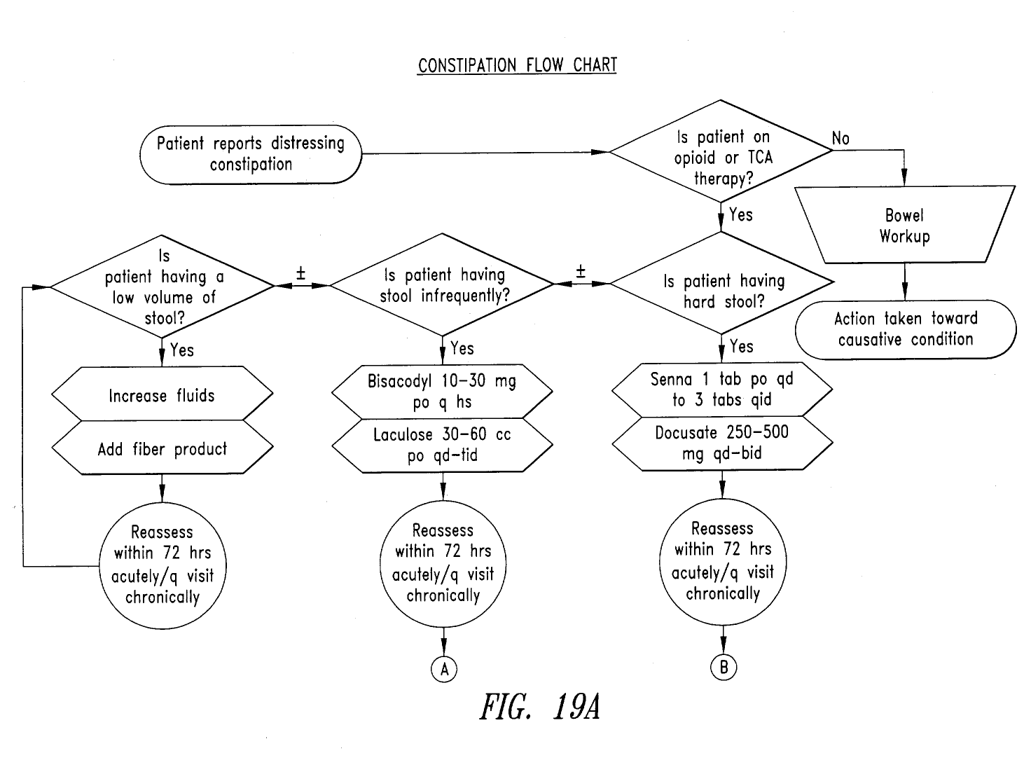

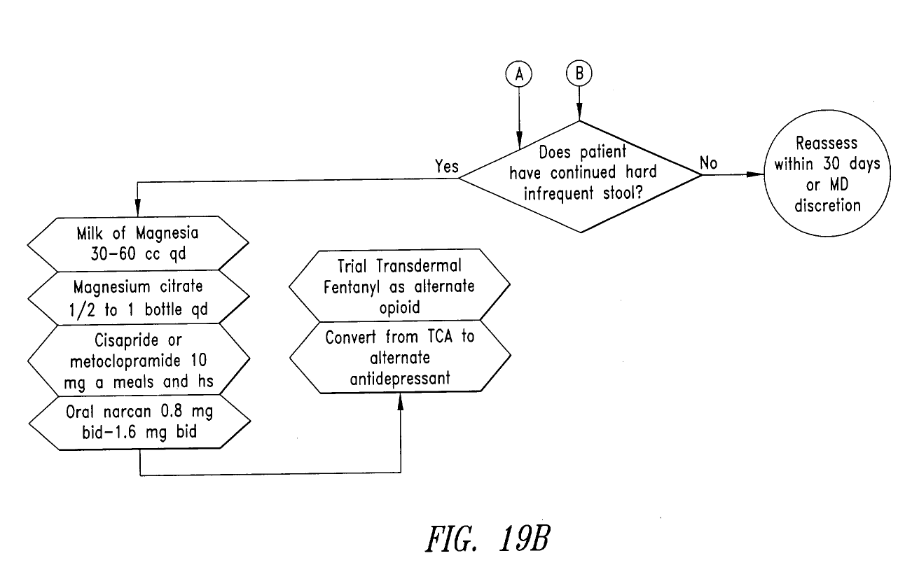

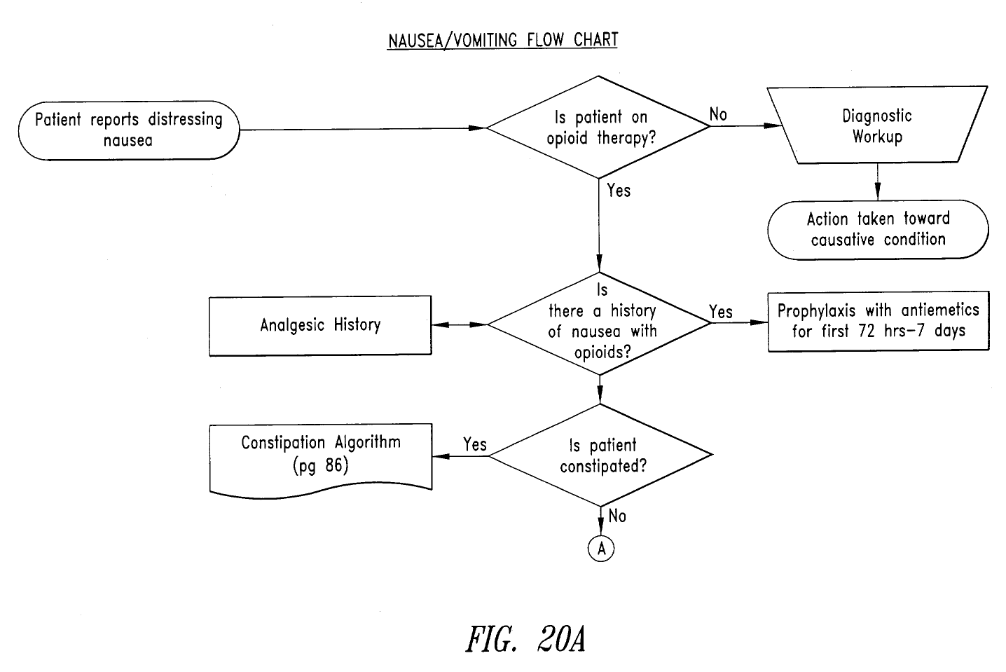

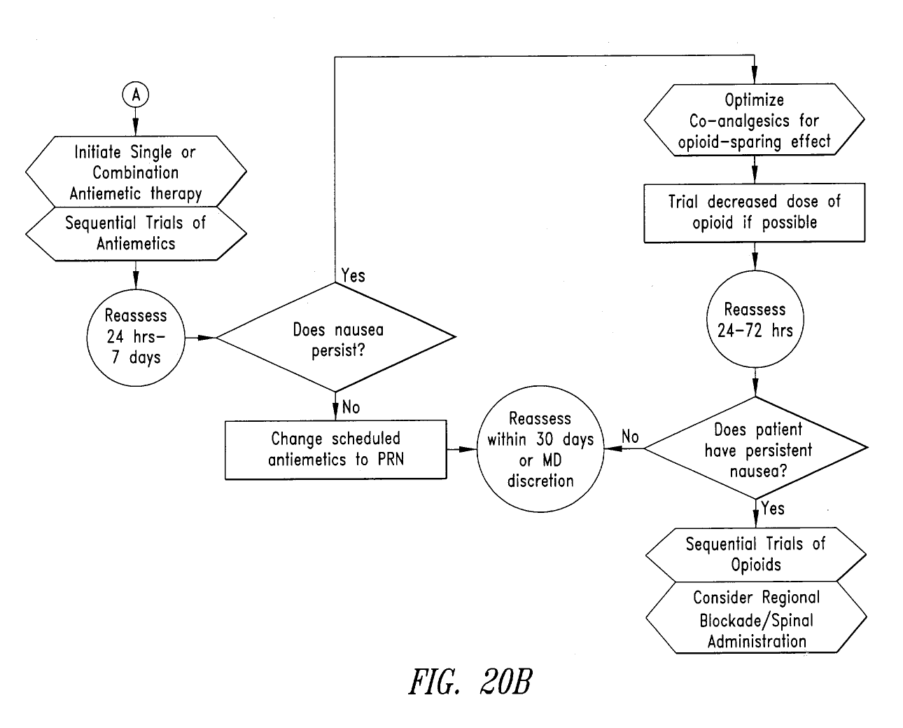

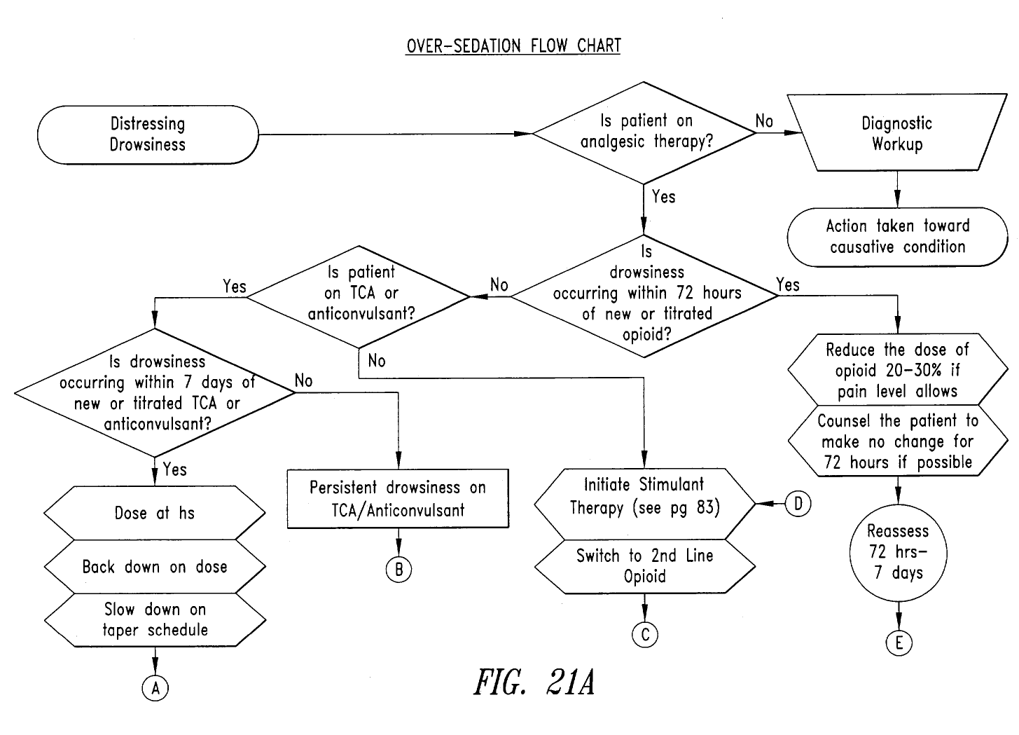

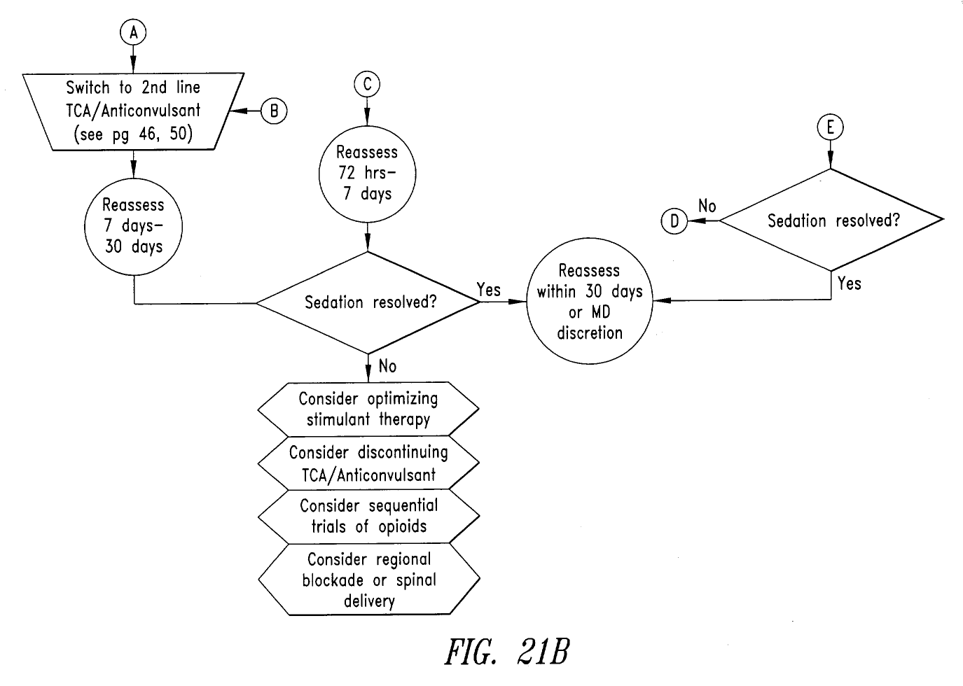

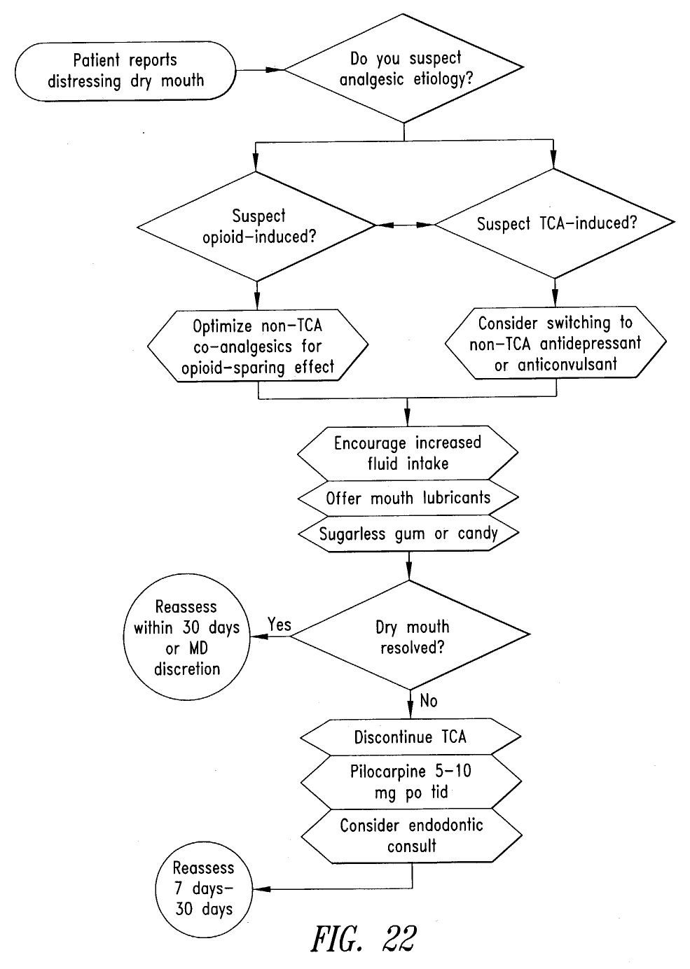

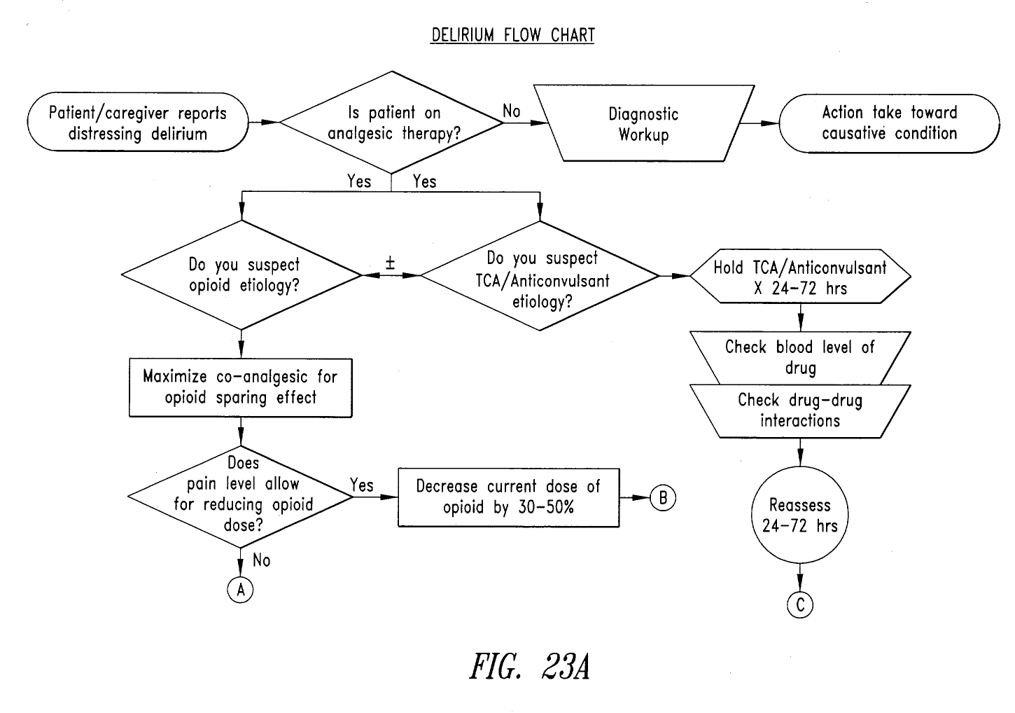

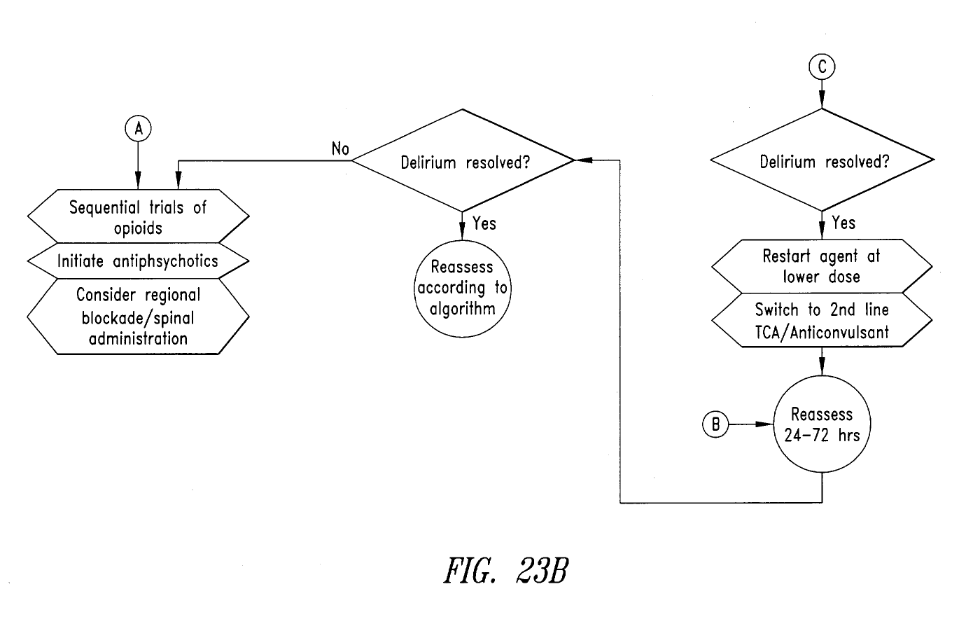

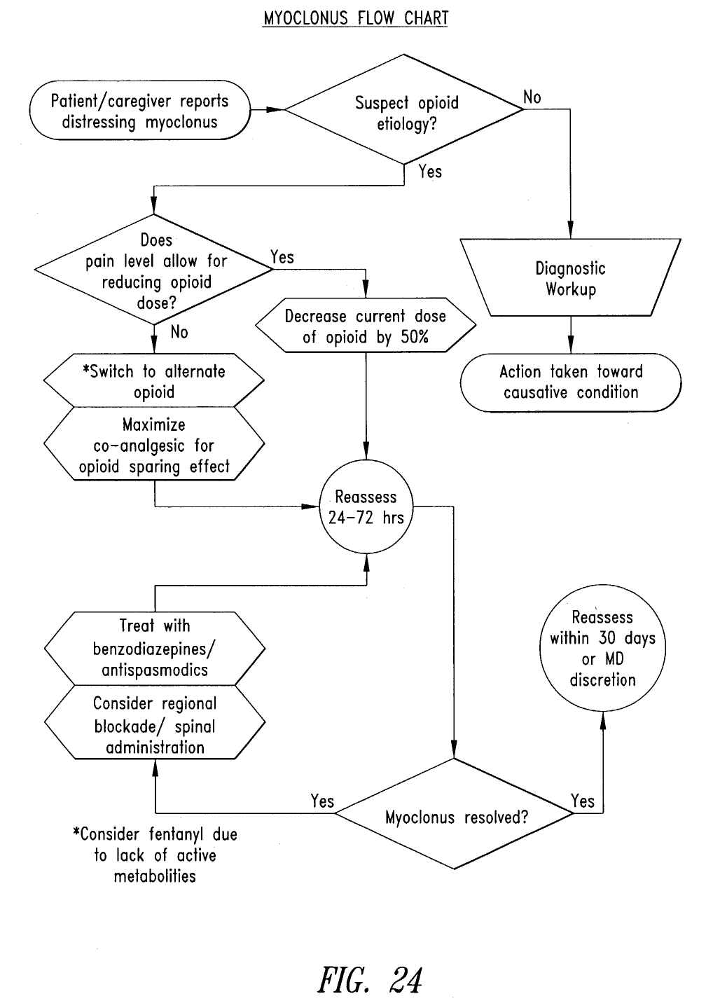

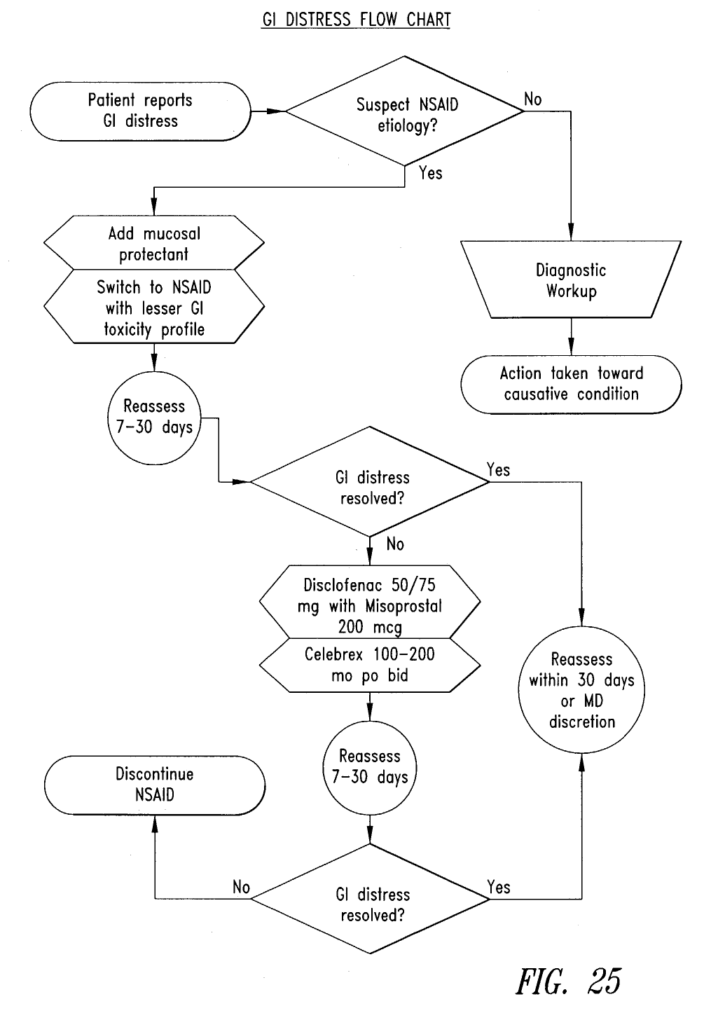

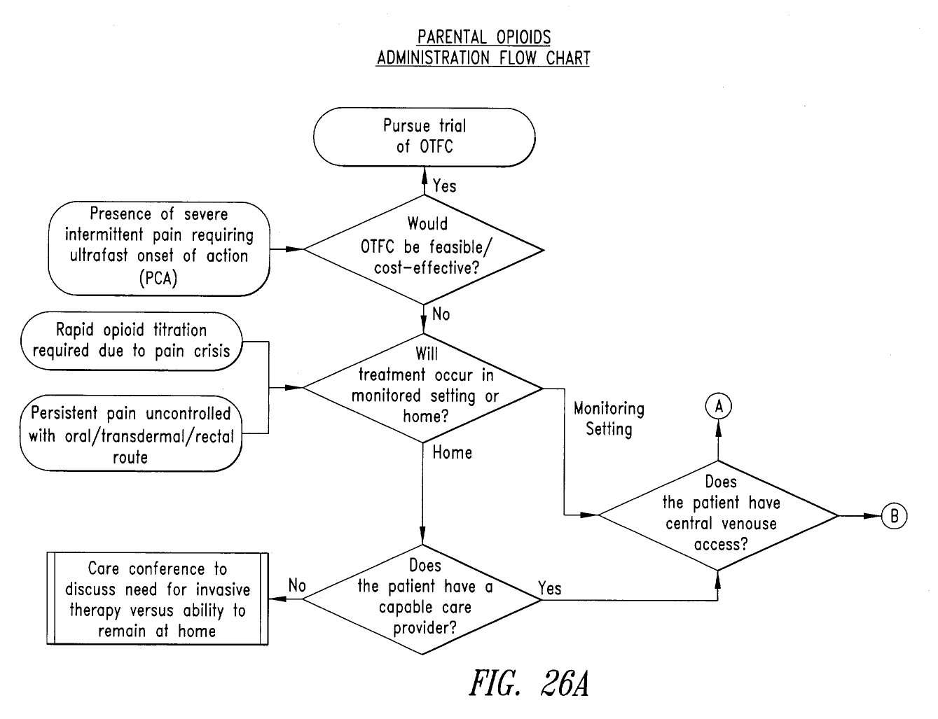

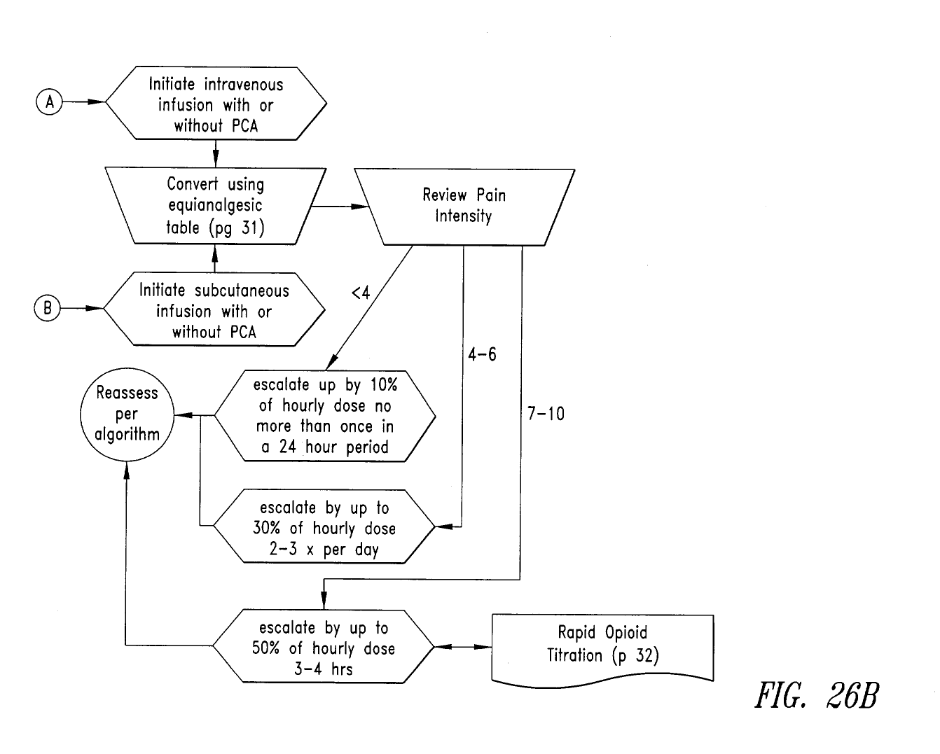

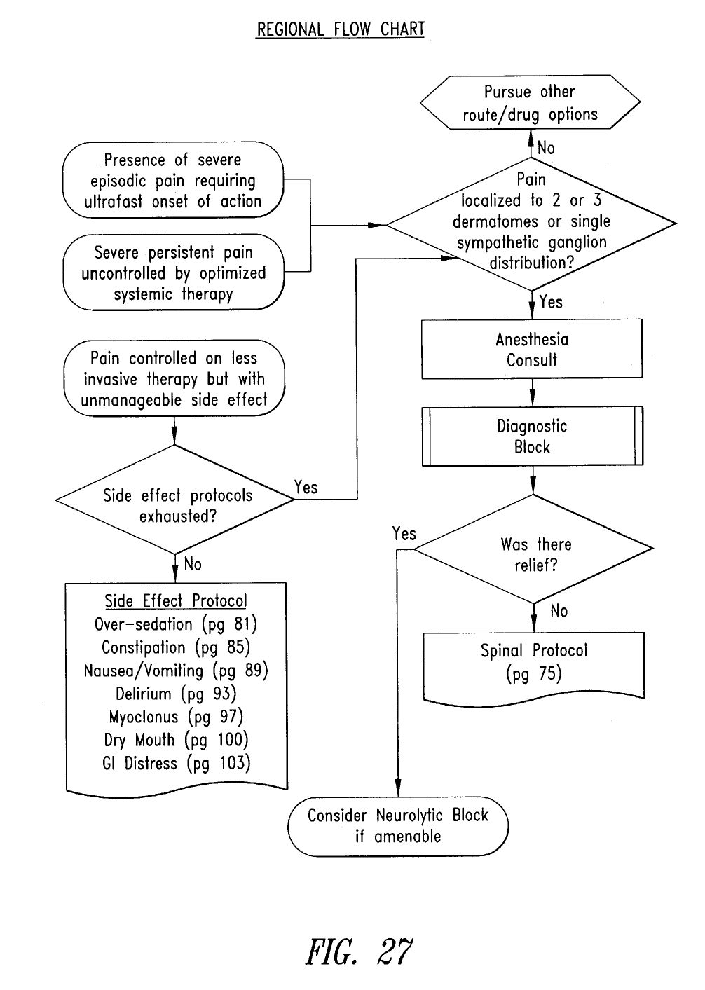

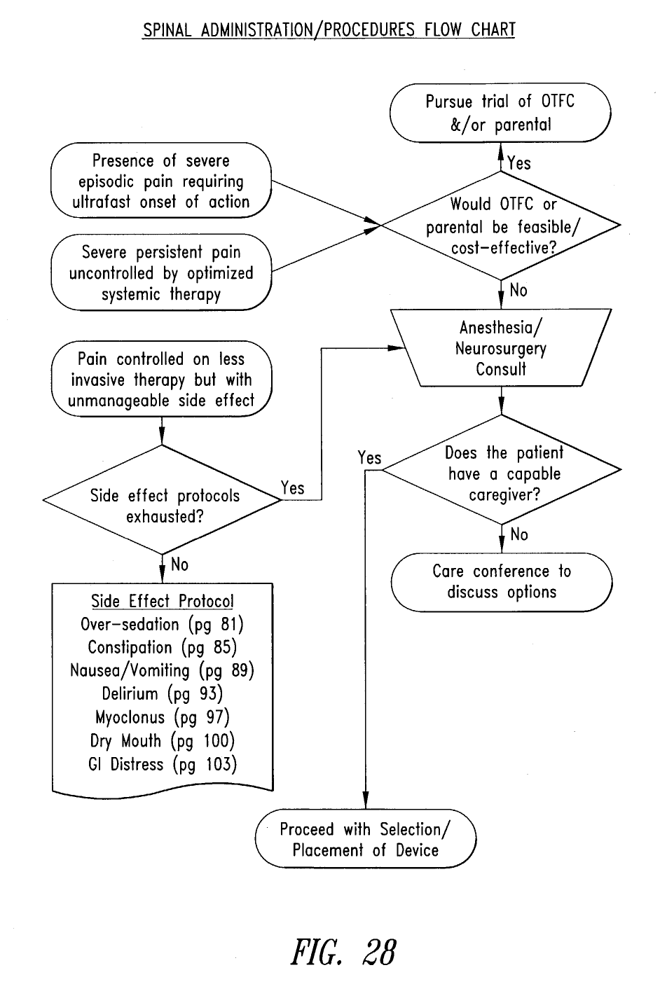

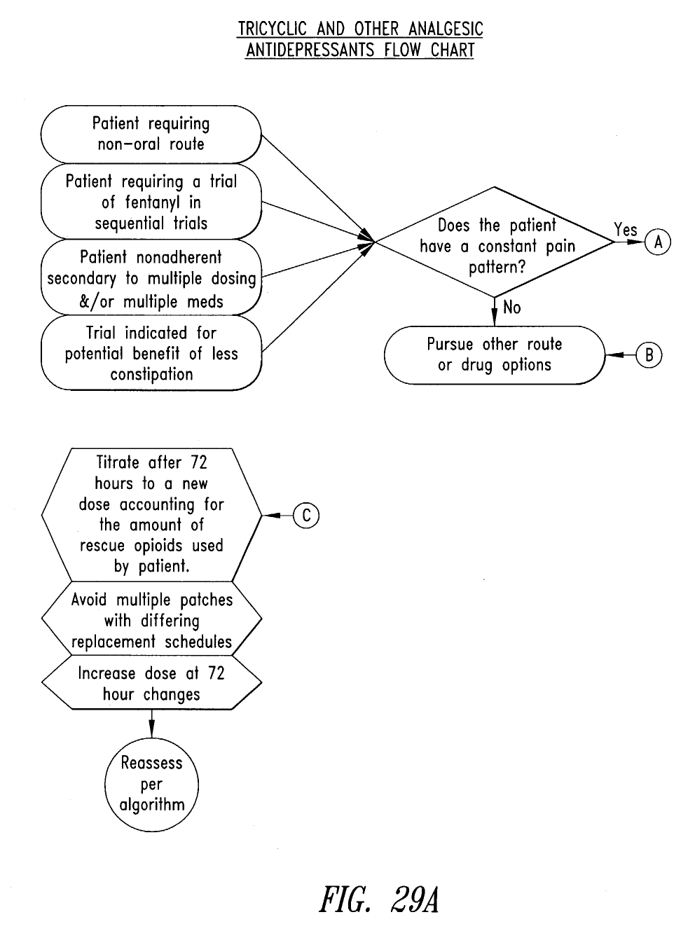

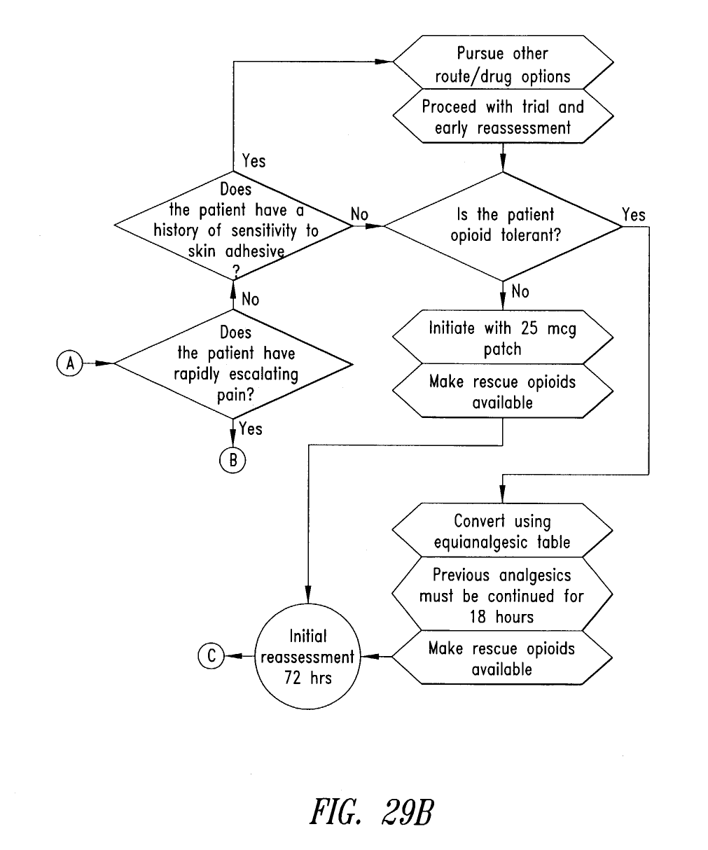

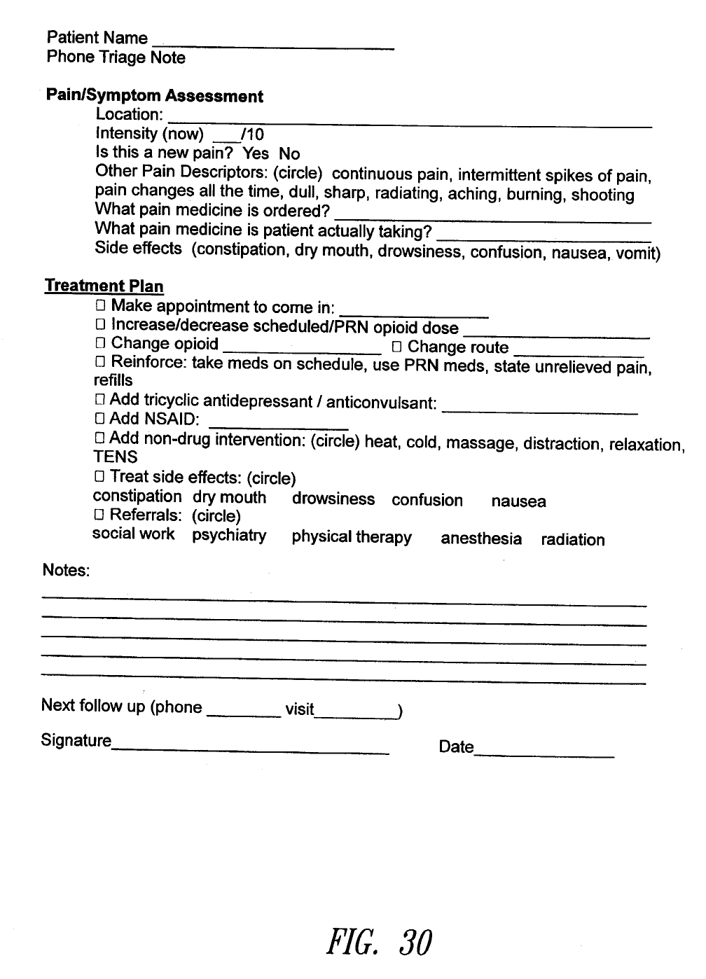

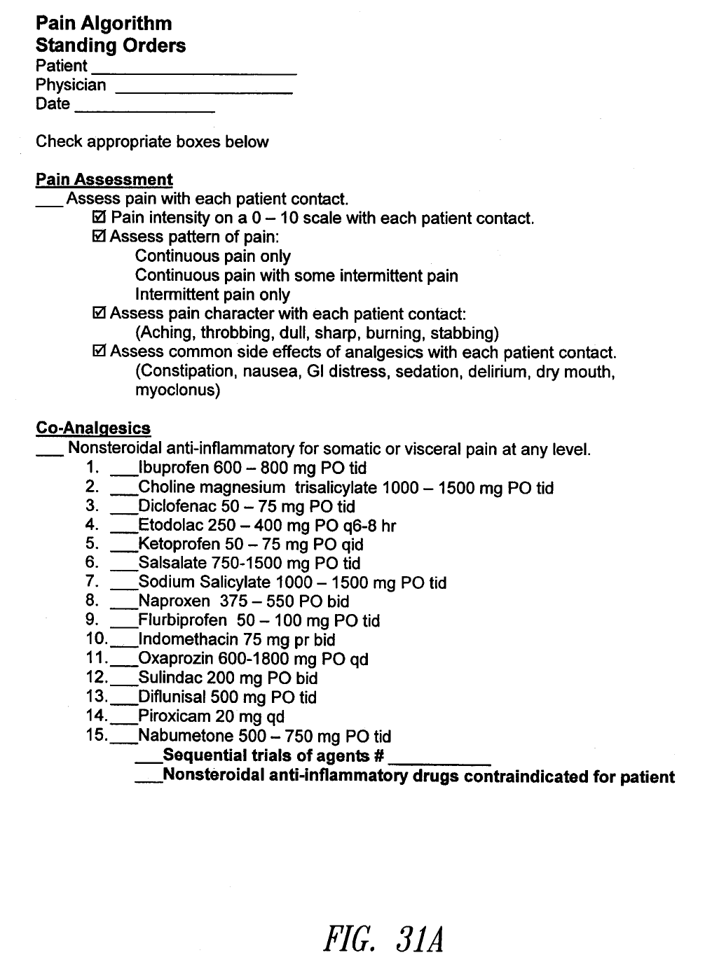

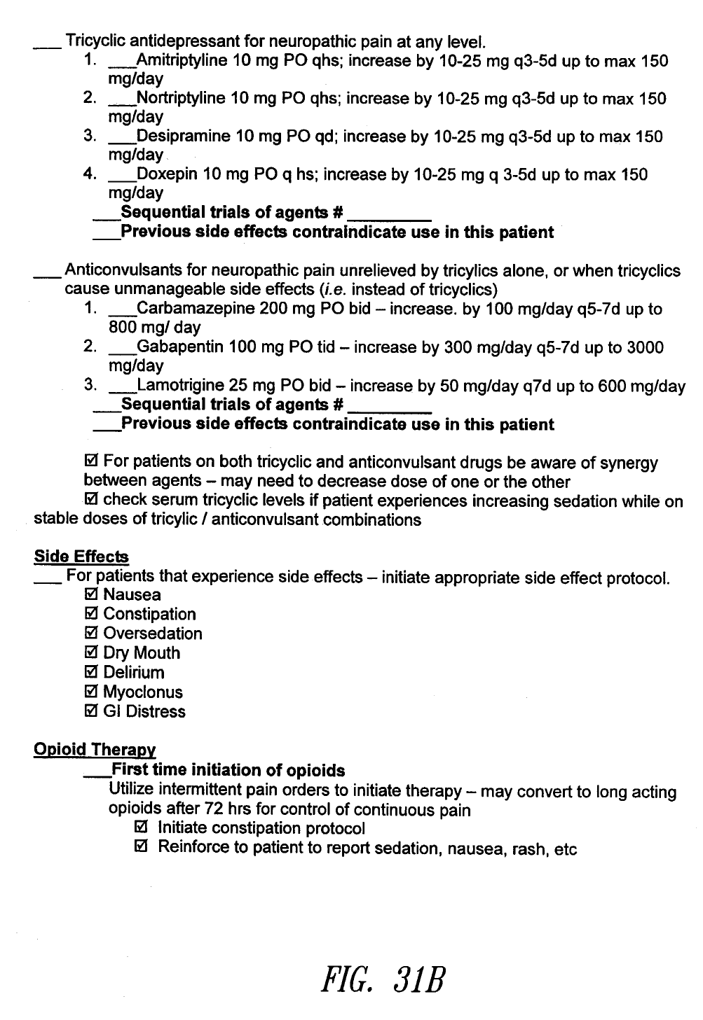

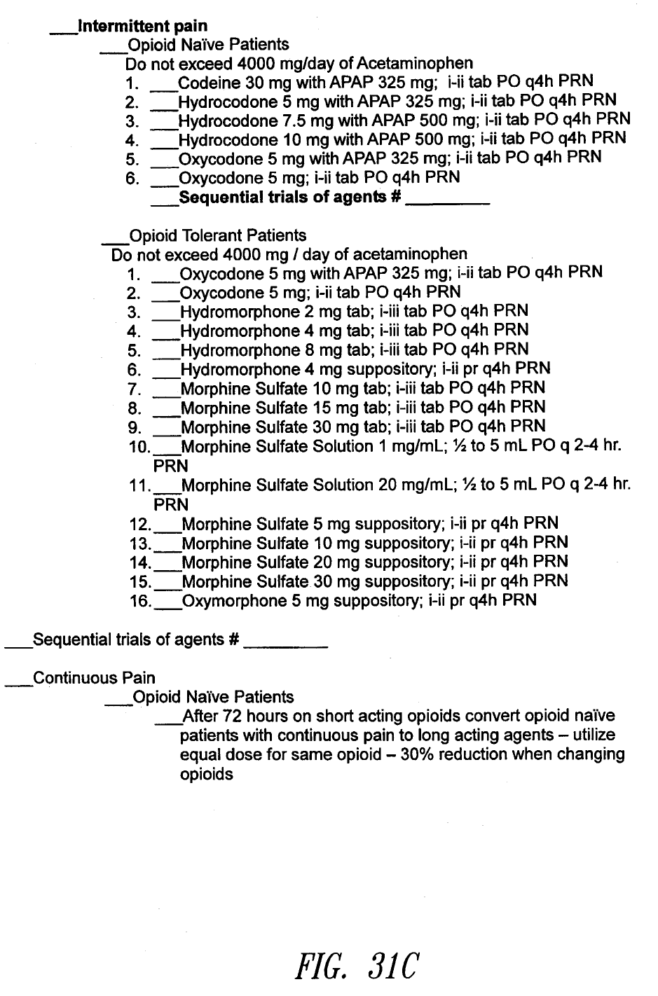

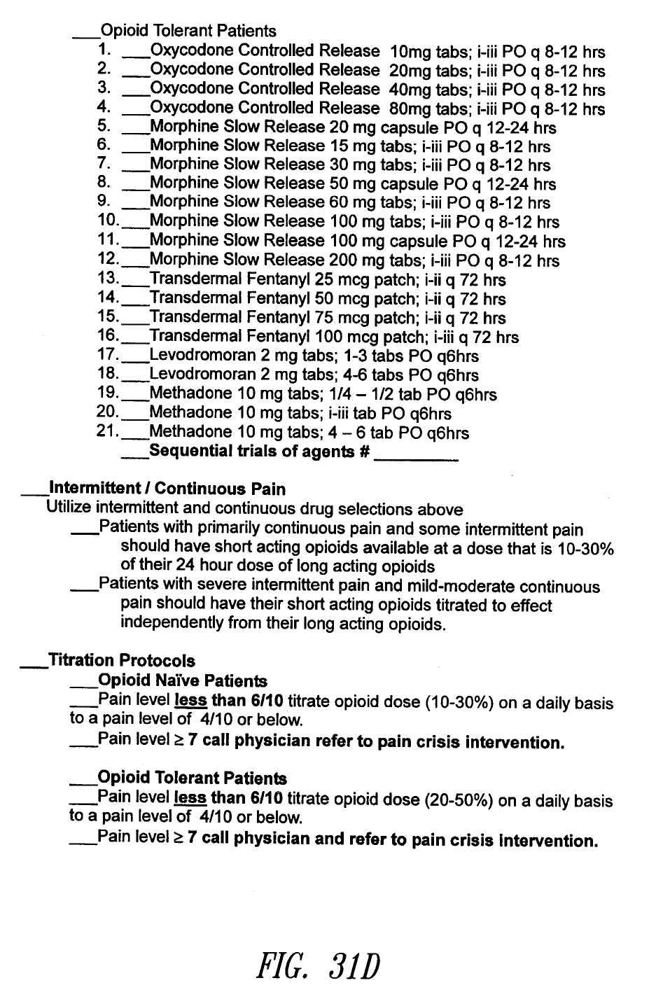

### DETAILED DESCRIPTION OF THE INVENTION

The system for pain management and the pain algorithm uses assessment to guide analgesic treatment decisions. It provides guidelines for opioid management and encourages the use of co-analgesics. Pain intensity guides the aggressive use of opioid titration, conversion, and use of alternative routes. Pain character guides the judicious use of nonsteroidal anti-inflammatory drugs, tricyclic antidepressants, and anticonvulsants. Consistent pain assessment is mandatory. Patient's priorities and perspectives need to be integrated into treatment planning. Congruently patients and caregivers need to accept responsibility for tracking and reporting pain and side effects promptly. Providers and institutions will need to make a commitment to cancer pain management—be available and responsive—in order to be successful.

Algorithm Essentials

Don't assume the patient is taking their pain medication—always ask!

Fear of addiction is a major barrier—early education is most beneficial

Ask the patient what level of pain would be considered tolerable for them—this will help to clarify issues that relate to pain relief versus side effects.

Whenever possible utilize around the clock dosing—it takes less drug to prevent pain than it does to treat it.

Always, always, always prescribe something for constipation

Cancer pain can be managed effectively through relatively simple means in up to 90% of patients with cancer or a history of cancer. Unfortunately, pain associated with cancer is frequently under-treated. Flexibility is the key to managing cancer pain. Each patient responds differently to pain, to the interventions for pain, and has personal preferences regarding treatment. In order to meet the unique needs of each patient, it is crucial that the plan be individualized. The patient will be most likely to follow a plan that suits his or her individual needs.

Pain Assessment

Analgesic History

An initial pain assessment may be lengthy, however it is crucial in setting the stage for future assessment and reassessment. Once the patient, caregiver and provider establish an understanding of the type of pain an individual is experiencing, and the ways in which the pain varies, the assessment process becomes a natural part of each visit or telephone communication. Staying ahead of the pain, treating episodes of exacerbation of pain and recognizing new pain will lead to a more effective and often less complex pain management plan. An initial assessment leads to the development of a pain management plan. Subsequent assessments will evaluate the effectiveness of the plan. Changes to the plan, or further investigation of pain may be needed if pain continues without relief.

Analgesic History Tool

| Please list the medications | | | | | |
|---|---|---|---|---|---|
| you are taking or have taken | | | | Did it cause | |
| for pain in the past: | | Did it work? | | problems?* | |
| Name of medicine | Dose | ✓ the answer | | ✓ the answer | |
| 1 | | Yes | No | Yes | No |
| 2 | | Yes | No | Yes | No |
| 3 | | Yes | No | Yes | No |
| 4 | | Yes | No | Yes | No |
| 5 | | Yes | No | Yes | No |
| 6 | | Yes | No | Yes | No |
| 7 | | Yes | No | Yes | No |
| 8 | | Yes | No | Yes | No |

*If you answered Yes to “Did it cause problems?” please descibe the problem you had with that particular drug in the space provided below:
Medicine #
Medicine #
Medicine #
Medicine #
Medicine #
Medicine #
Medicine #

Pain is always a subjective experience. Individuals learn the meaning of the word “pain” through their past experiences, and by observation of the pain that others experience. Pain may be constant or last only a short time (duration), severe or mild (intensity), aching or burning (character), brought on by one activity and relieved by another. Other factors such as anxiety and depression contribute to a person's pain experience.

Patients tell us that pain is very hard to describe. It is important for the patient, family and health care team to speak the same language. The things to communicate are what the pain feels like, how much it hurts, and where it hurts.

Pain Location

Patients will likely have more than one pain site (there were an average of five sites reported by patients in the algorithm study). It is important to identify all sites, with corresponding intensity and character. A combination of therapies is often required to alleviate the different types of pain that an individual patient is experiencing, and/ or to minimize bothersome side effects. Any time a new pain is described, it should be explored, as it may indicate either a new site of involvement that may benefit from certain therapies not yet being utilized.

TABLE 1
THE BASICS OF PAIN ASSESSMENT

| Question | Tool | Rationale |
|---|---|---|
| Where is the | Body | Is the location of the pain consistent |
| pain? | template | with the known disease? |
| Is this a new | Interview | If yes, need to alert the MD |
| pain? | | |
| Is the pain | Pain Pattern | The pattern of the pain leads us to what |
| constant, | Graphics | and how to titrate |
| episodic, or | | |
| some of | | |
| both? | | |
| What is the | 0-10 scale | Moderate to severe constant pain |
| level of | | should be treated with long acting |
| your | | agents |
| constant or | | |
| “usual” | | |
| pain? | | |
| What is the | 0-10 scale | Moderate to severe episodic pain |
| level of | | should be treated with short acting |
| your | | agents |
| episodic or | | |
| “worst” | | |
| pain? | | |
| What does | Pain character | The character of the pain leads to |
| the pain feel | word list | appropriate selection of co-analgesics |
| like? | | |
| What brings | Interview | Exacerbating factors can be treated |
| on the pain? | | proactively |
| What | Interview | Relieving factors can be explored, |
| relieves the | | particularly nonpharmacologic avenues |
| pain? | | |
| Are you | Side Effects | Side effects require treatment or drug |
| having side | Questionnaire | change |
| effects of | | |
| your current | | |
| medication? | | |

Pain Pattern

Patients commonly have a combination of constant and episodic pain. Analgesic therapy needs to be tailored to the pattern of the pain. For example, treating high peaks of episodic pain with long acting agents will often cause significant side effects during the periods between the peaks. Patients with primarily episodic pain require distinctly different titration protocols than patients with primarily constant pain. You can use the following figures to analyze whether the pain is continuous or episodic, and can use them as an aid to help the patient describe their patterns of pain to you.

Pain Intensity

The use of a pain scale is an important part of the pain assessment. There are different scales available to help patients understand the concept of a pain scale. Some patients prefer descriptive words, or color scales. The standard scale is the 0-10 scale. It is important to use one scale. If you change between a 0-5 and 0-10 scale, for example, note that a 5/5 is much different than a 5/10. Be consistent.

Have the patient describe the pain over a specific period of time (i.e. last 24 hours) in terms of:

the worst that the pain gets

the least amount of pain they experience

the usual pain level or what the pain is like “most of the time”

When teaching the patient about the pain scale, spend time learning about the individual meaning of each person's scale. Many patients experience extreme “discomfort”, yet will deny “pain”. Again, it is important to use language that the patient understands.

Another important factor is what the patient considers a tolerable level of pain. Often the patient will decide whether or not to take medication based on what they think is an acceptable or tolerable level of pain. Patient and provider should set a goal to try for at least the tolerable level for that individual patient. Patients in the algorithm study (mean for the group) reported that a “4” on a scale of 0-10 was a tolerable level. The whole issue of pain tolerability often brings up the discussion of pain versus toxicity (i.e. what if 0/10 is not possible without some side effects). It is extremely helpful to have this discussion early so that the patient's preferences are known and patient education regarding the potential efficacy to toxicity ratio of treatment is defined for the patient.

Even with the help of pain tools, many patients will still find it difficult to rate their pain. Mild, moderate or severe may be the only way the patient can verbalize the pain he or she is experiencing. If the patient is nonverbal, note nonverbal signs such as grimacing or moaning. Be careful not to assume that the patient is without pain if nonverbal cues are absent. Many patients with chronic pain will not show outward signs of pain, despite the fact that the feel pain.

Character

Identifying the pain “generators” or source (tissues involved) is a key to treating pain. We can not open the patient up every time we do a pain assessment to see if nerve or bone or soft tissues are being pressed on by tumor. The best thing we have is the patient's description of what the pain feels like. Have the patient describe each site of pain using the words below.

| | | | | |
|---|---|---|---|---|
| Neuropathic Pain | aching | shooting | sharp | tender |
| Nociceptive Pain | cramping | burning | stabbing | throbbing |

The descriptors help guide decisions regarding whether or not the pain is nociceptive, neuropathic, or mixed. If the pain character the patient describes only comes from the top row, then the pain is neuropathic. If the pain character only comes from the bottom row, then the pain is nociceptive. If the patient chooses pain character descriptors from both the top and bottom row, then the pain is a mix of neuropathic and nociceptive. It is very clear from clinical trials that co-analgesics targeting the type of pain contribute to pain relief.

What Brings on the Pain?

Patients with bony involvement will often have motion-related pain. Patients with GI tumors may have pain that is exacerbated by certain foods. Some chemotherapeutic agents will bring on joint pain. Whenever possible, encourage the patient to medicate prior to pain causing events. Multiple studies have proven that it takes less medication to prevent pain than it does to treat pain that is out of control.

What Relieves the Pain?

Does the current regimen provide enough relief? Are there certain positions that the patient can get into that relieve some or all of the pain? Has the patient tried physical (hot/cold) or psychological (relaxation/imagery) techniques to supplement their pain relief?

TABLE 2
Side Effects Questionaire

| Side Effect | | | If yes, is this problem. . . |
|---|---|---|---|
| Are you | YES | NO | □ mild, not really a problem |
| experiencing | | | □ moderate, a significant problem |
| nausea? | | | □ SEVERE, A VERY DISTRESSING PROBLEM |
| Are you | YES | NO | □ mold, not really aproblem |
| having hard or | | | □ moderate, a significant problem |
| infrequent | | | □ SEVERE, A VERY DISTRESSING PROBLEM |
| bowel | | | |
| movements? | | | |
| Do you | YES | NO | □ mold, not really a problem |
| experience a | | | □ moderate, a significant problem |
| lot of | | | □ SEVERE, A VERY DISTRESSING PROBLEM |
| drowsiness | | | |
| during the | | | |
| day? | | | |
| Do you ever | YES | NO | □ mild, not really a problem |
| have bad | | | □ moderate, a significant problem |
| dreams or see | | | □ SEVERE, A VERY DISTRESSING PROBLEM |
| things that | | | |
| aren't there? | | | |
| Does your | YES | NO | □ mild, not really a problem |
| stomach hurt | | | □ moderate, a significant problem |
| after you take | | | □ SEVERE, A VERY DISTRESSING PROBLEM |
| your pills? | | | |
| Are you | YES | NO | □ mild, not really a problem |
| experiencing | | | □ moderate, a significant problem |
| muscle | | | □ SEVERE, A VERY DISTRESSING PROBLEM |
| twitches or | | | |
| jerks? | | | |
| Are you | YES | NO | □ mild, not really a problem |
| experiencing | | | □ moderate, a significant problem |
| dry mouth? | | | □ SEVERE, A VERY DISTRESSING PROBLEM |

| | |
|---|---|
| FOR THE SIDE EFFECTS THAT | 1. |
| ARE MODERATE OR SEVERLY | |
| DISTRESSING WHICH ONE OR | |
| TWO SHOULD BE THE TOP | |
| PRIORITY FOR TREATMENT | 2. |
| TODAY? | |

Side effects associated with opioids usually disappear within three days of starting a new medication or increasing the dose. However, many patients find these side effects so bothersome that they would rather stop the medicine. It is therefore important to spend time with patients at the outset of treatment discussing possible side effects they may develop, the transient nature of most side effects, and treatments available to minimize the side effects during the first few days of treatment.

Some patients report an “allergy” to an opioid because of side effects such as nausea or vomiting. This is not an allergy, but a side effect. Prophylactic treatment with an antiemetic for the first few days might allow the patient to continue with a medication that provides pain relief that they would otherwise refuse to take.

All patients must have a bowel regime prescribed for them. Consider individual response to the constipating effects of the medication. Once the patient understands what constitutes constipation (no adequate bowel movement >2 days), he/she will be able to manage their bowel regime accordingly. Frequent assessment will minimize deleterious effects in the beginning of treatment. Again, flexibility is the key.

Side Effects of Current Pain Treatment

Cancer patients often experience side effects related to treatments or pain medications. Side effects commonly associated with medications for pain include:

Constipation

Nausea and Vomiting

Oversedation

GI Distress (Indigestion, Heartburn, Sour Stomach)

Dry Mouth

Myoclonus*

Delirium*

Constipation can be treated with fluids and a high fiber diet, as well as stool softeners and emolient laxatives in combination with stimulant laxatives.

Nausea and Vomiting can be treated with aggressive titration. Finding the proper pain drug and/or the proper dosage levels can make all the difference for a patient. To find a medication level where the patient has acceptable pain and tolerable nausea is important, for repeated vomiting can cause dehydration and may complicate existing problems.

Oversedation

If sedation is occurring at the initiation of opioid, antidepressant, or anticonvulsant therapy, wait at least 72 hours to allow the patient to gain tolerance. Sedation in relation to antidepressant or anticonvulsant therapy may take longer than 72 hours; this can be minimized by starting the patient with lower doses and titrating slowly to the analgesic effect.

GI Distress

NSAIDS commonly cause GI Distress. Treatment options include changing to other NSAIDS that have a reportedly less GI toxicity, such as salsalate, choline magnesium trisalicylate, and nabumetone. Diclofenac/misoprostol combination product can be useful, though it is contraindicated in pregnant women.

Dry Mouth

Unless contraindicated, try to encourage fluid intake and the use of mouth lubricants. Sugarless gum or candy is an excellent means to treat dry mouth. For more severe cases, pilocarpine could be considered to relieve this side effect.

(*Myoclonus and delirium are generally exceptions to this list, and may require urgent evaluation if either of these side effects manifest.)

[image]

Sample Case Studies: Pain Assessment

Case #1

Mrs. L., a 65 y.o. white female diagnosed with breast ca (dx in 1995), is referred to hospice. Current metastatic sites include bone (thoracic and lumbar spine, right femur). Past medical history was significant for cholecystectomy, appendectomy, and arthritis (bilat knees, hands). She is s/p mastectomy, XRT, chemo-(taxol).

On admission, the patient reports pain located in her upper and lower back, and RLE. She rates her back pain as: worst 9/10, least 3/10, usual 7/10. She rates her RLE pain as: worst 9/10, least 1/10, usual 3/10. She described the pain in her upper and lower back as “aching”, and “sharp”. She also has a pain originating in her lower back, radiating down her RLE which she describes as an “electric shock”. Her pain is constant in all places, with increases in intensity at each site with movement. The pain is relieved by rest and medicine. She has not had a bowel movement in 2 days.

Medications:

Percocet 1-2 tabs q 4 h prn

Ibuprofen 400 mg q 6 h prn.

Colace 100 mg qd

She takes ˜5 Percocet per day, and 2-4 Ibuprofen per day. She reports that the percocet makes her feel nauseated, and that the Ibuprofen doesn't seem to work.

QUESTIONS

1. PROBABLE ETIOLOGY OF PAIN (TUMOR-RELATED, TREATMENT-RELATED, NON-MALIGNANT)

2. SITES

3. PAIN INTENSITY

4. PAIN CHARACTER (NOCICEPTIVE, NEUROPATHIC, MIXED)

5. WHAT BRINGS ON THE PAIN?

6. WHAT RELIEVES THE PAIN?

7. SIDE EFFECTS

8. DRUG DECISIONS

Case #1 (Continued)

Of 12 percocet possible/day—she takes 5

Patient was instructed to take percocet on a “regular schedule”: 2 tabs q 4 h ATC while awake (may take during the night if necessary). Also, she is instructed to take her Ibuprofen on a schedule: q6h with food. An order is obtained to increase the Colace to TID as needed. Senokot BID as needed is added. Compazine 10 mg q 6 h prn is ordered.

24 hours later, Mrs.L reports she took 10 percocets in the last 24 hours. Her “usual” pain is at a 6/10 (aching in her upper & lower back), and usual leg pain at a 1-2/10, (shooting). She had a large BM last PM and a medium, soft BM this morning. She is drowsy. The nausea persists, but is a little better after taking 2 compazines.

*Start MS Contin; consider MS IR or keep on percocet for breakthrough; cont. ibuprofen ATC; consider something for her stomach—pepcid

*TCA or anticonvulsant could be added if neuropathic pain not relieved by opioid.

Opioids

A patient is a candidate for opioid therapy if there is the presence of severe cancer pain, if they have been trialed on maximal doses of co-analgesic Rx, and if there are dose limiting side effects of co-analgesic Rx. Major questions the clinician should answer deal directly with the pattern of the patient's pain, whether it is continuous, a combination of continuous and episodic, or is primarily episodic.

The patient's goals for therapy should be carefully assessed. Issues to be addressed by the clinician include the patient's willingness to accept treatment with opioids, and ensuring the patient's education on their responsibility to report the effect of all interventions. It should be discovered if the patient has received opioids in the past, as previous opioid experience might influence the current dosing and titration process.

Always, always, always utilize a preventive bowel regimen, and titrate carefully to reach efficacy and to treat side effects. (Efficacy=pain level≦4)

Patients that are taking 60 mg of morphine each day, 50 mcg of transdermal fentanyl each hour, or an equianalgesic dose of another opioid for a week of longer care are considered opioid tolerant. All other patients not matching these criteria are considered to be opioid naïve.

Guiding Principles

Significant barriers to opioid therapy exist for both the patient and the provider; frequent re-assessment, patient education, simplification of treatment regimens, cost control, and continuous negotiation are key factors in a successful opioid treatment plan.

The primary challenge of opioid therapy is to titrate to a balance of efficacy versus side effects. Pain intensity drives the aggressiveness of opioid therapy; the simplest dosage schedules and least invasive modalities should be utilized first. As morphine is the standard drug in the opioid category (a known narcotic) many clinicians and patients confuse addiction with tolerance. (Addiction is defined as taking medications to satisfy emotional or psychological needs rather than to relieve pain Tolerance is present in a patient when larger doses of opioids are required to maintain the same level of pain relief.)

Cancer pain requires an “around the clock” approach. A single agent given on a schedule to create a “baseline” of analgesia is highly recommended. Patients who are opioid naïve are those that have not yet established an opioid medication level that provides a stable analgesic effect. Patients who are opioid tolerant are generally defined as those who are taking at least 60 mg morphine day, 50 mcg transdermal fentanyl/hour, or an equianalgesic dose of another opioid for a week or longer.

For patients requiring more than six doses of short acting medication a day, consider low dose long acting. Studies show that q8-12 hr dosing enhances compliance when compared to q4-6 hr dosing. Patients with primarily continuous or combination continuous/intermittent pain should have “rescue” opioids dosed at 10-30% of the 24 hour total dose. Patients with primarily intermittent pain may require very high doses of fast acting, “rescue” medication with no relationship at all to a long acting dose. These patients are at high risk for intractable pain.

Drug Choices

Initiate treatment with current fast acting opioid through whatever route available.

If patient is having unmanageable side effects go to sequential opioid trials.

Opioid Drug Options and Pain Management
MANAGEMENT OPTIONS FOR
MILD TO MODERATE EPISODIC PAIN

| | |
|---|---|
| For mild pain (1-4): | Add or optimize co-analgesic |
| | Initiate/escalate short acting opioid |
| For moderate pain (5-6): | Add or optimize co-analgesic |

DRUG CHOICES FOR EPISODIC PAIN
(FOR OPIOID NAIVE PATIENTS)

Do not exceed 4000 mg/day of acetaminophen in combination preparations
Codeine 15-30 mg with APAP 325 mg; i-ii tab po q4h prn
Hydrocodone 5 mg with APAP 325 mg; i-ii tab po q4h prn
Hydrocodone 5 mg with ibuprofen 200 mg, i-ii tab po q4h prn
Oxycodone 5 mg with APAP 325 mg; i-ii tab po q4h prn
Oxycodone 5 mg with APAP 500 mg; i-ii tab po q4h prn
Oxycodone 5 mg with ASA 325 mg; i-ii tab po q4h prn
Oxycodone 5 mg; i-ii tab po q4h prn
Tramadol (max dose 400 mg/day) 50-100 mg; I-ii tab po q4h prn

DRUG CHOICES FOR EPISODIC PAIN
(FOR OPIOID TOLERANT PATIENTS)

Do not exceed 4000 mg/day of acetaminophen in combination preparations
Hydrocodone 7.5 mg with APAP 500 mg; i-ii tab po q4h prn
Hydrocodone 10 mg with APAP 500 mg; i-ii tab po q4h prn
Oxycodone 5 mg; with APAP 325 mg; i-ii tab po q4h prn
Oxycodone 5 mg; i-iii tab po q4h prn
Hydromorphone 2. 4, 8 mg; i-iii tab po q4h prn
Morphine Sulfate 10, 15, 30 mg; i-iii tab po q4h prn
Morphine Sulfate Solution 1-20 mg/mL; ½-5 ml po q4h prn
Oral Transmucosal Fentanyl 200, 400, 600, 800, 1200, 1600 μg; one unit
dissolved q1h prn
Patients with severe episodic pain are high-risk patients due to the
difficulty of managing this pain pattern. Titrate short acting opioids to
effect independently from any long acting opioids. The clin-
ician should research the situation carefully so they can pre-medicate for
predictable incident pain and can explore co-analgesic drugs aggressively.
Severe episodic pain (pathological fx. Plexus invasion) may require re-
gional anesthesia techniques.

MANAGEMENT OPTIONS FOR
MILD TO MODERATE EPISODIC PAIN

| | |
|---|---|
| For mild pain: | Add or optimize co-analgesic |
| (1-4) | Initiate/escalate short acting opioid |
| For moderate pain: | Add or optimize co-analgesic |
| (5-6) | |

MANAGEMENT OPTIONS FOR
SEVERE EPISODIC PAIN

| | |
|---|---|
| For Severe Pain: | These are high-risk patients due to the diff- |
| (7-10) | iculty of managing this pain pattern. |
| | Titrate short acting opioids to effect inde- |
| | pendently from any long acting opioids. |
| | Pre-medicate for predictable incident pain |
| | Explore co-analgesic drugs aggresively |
| | Severe episodic pain (pathological fracture, |
| | plexus invasion) may require regional |
| | anesthesia techniques. |

Example: For an opioid naive patient with moderate, intermittent pain, initiate the patient with 5 mg of oxycodone with APAP 325 mg; i-ii tab PO q4 h prn. Instruct the patient to monitor for early side effects that will likely only be transient; instruct the patient that they should contact the clinician if the pain pattern becomes continuous rather than episodic.

Opioid Drug choices for Continuous pain
DRUG CHOICES FOR CONTINUOUS PAIN
FOR OPIOID NAIVE PATIENTS

After 72 hours on short acting opioids convert opioid naive patients with
continuous pain to long acting agents. Utilize an equal dose for same
opioid. Be sure to calculate a 30% reduction when changing opioids.

DRUG CHOICES FOR CONTINUOUS PAIN
FOR OPIOID TOLERANT PATIENTS

| | | |
|---|---|---|
| Oxycodone Controlled Release | 10, 20, 40 or 80 mg tabs | q 8-12 hrs |
| Morphine Slow Release | 20, 50 or 100 mg | q 12-24 hrs |
| | capsules | |
| Morphine Slow Release | 15, 30, 60, 100 or 200 | q 8-12 hrs |
| | mg tabs | |
| Duragesic (see transdermal | 25, 50, 75, 100 μg | q 72 hrs |
| route) | patches | |
| Levo-Dromoran | 2 mg tabs | q 6 hrs |
| Methadone | 10 mg tabs | q 6 hrs |

Example: A patient has been on 120 mg of sustained release morphine q12h with 45 mg of IR MS for breakthrough and now has rapidly increasing pain (no side effects)—the total morphine dose for the last 24 hours=330 mg. You initiate the pain crisis intervention by giving a loading dose equivalent to 25% of the previous 24 hour dose—(˜80 mg)—forty five minutes later the pain level is down to 3 out of 10 with no sedation. Your plan is to continue the 80 mg, rescue dose for the next 6-12 hours and then recalculate the new dose of ATC drug—within the next 12 hour the patient uses three more doses of 80 mg. The patient has only mild sedation with no other side effect—you add up the last 24 hr dose and find it to be 520 mg—the new ATC dose should be 240-260 mg q 12 hr with a rescue dose in the range of 50-150 mg (10-30%)

Management Options For Combined Continuous/Episodic Pain

The clinician should calculate the twenty four hour dose of opioid utilized for continuous pain. Patients with primarily continuous pain with some intermittent pain should have rescue doses of 10%-30% of the twenty four-hour dose. The chief goals are to maintain a constant pain level of or below a 4 out of 10 utilizing titration. When using sustained release opioids avoid increasing the frequency, and try to increase the dosage instead.

The patient's pain intensity should guide the aggressiveness of the titration. Opioid doses may be titrated downward by 25-50% of the daily dose every one to two days until the daily scheduled medication is equivalent to 15 mg of morphine.

Discontinuation of opioids at higher doses can cause symptoms of withdrawal. Sequential trials of opioids are indicated for persistent side effects and/or intractable pain. Patients receiving anti-tumor therapy or regional techniques may experience decreasing pain and a sudden increase in side effects related to pain medication.

MANAGEMENT OPTIONS FOR CONTINUOUS PAIN

| | |
|---|---|
| Mild pain | Add or optimize co-analgesic |
| (1-4): | Convert PRN opioid to ATC |
| | Titrate ATC opioid dose by 10-20% |
| | Titrate ATC dose by 20-30% daily |
| Moderate pain | Changeto sustained release opioid |
| (5-6): | Add or increase co-analgesic |
| | Titrate ATC dose by 30-50% daily |
| Severe pain | Change to sustained release opioid |
| (7-10): | Change route of administration |
| | Initiate pain crisis intervention |

MANAGEMENT OPTIONS FOR
COMBINED CONTINUOUS AND INTERMITTENT PAIN
Utilize episodic and continuous drug selections above
Calculate the 24 hr dose of opioid utilized for continuous pain

| | |
|---|---|
| a. | Rescue dose for intermittent pain should be 10-30% of the 24 hour |
| | dose |

COMMONLY USED OPIOID ANALGESICS

| MEDICATION | ROUTES | 2. DRUG SPECIFIC INFORMATION |
|---|---|---|
| Morphine | PO, SQ, IM, | Morphine is the “standard drug” in the |
| | IV, PR, IS | opioid category. |
| Morphine )SR) | PO, PR | SR preparation-ATC scheduled use |
| | | only-don't crush or break-q8-12 hrs. |
| | | Kadian capsules (12-24 hr.) may be |
| | | opened and sprinkled over applesauce |
| | | or used in PEG tubes (see product in- |
| | | formation). |
| Hydromorphone | PO, SQ, IM, | 24 hour SR preparation to be |
| | IV, PR, IS | available in 1999. |
| MEDICATION | ROUTES | 3. DRUG SPECIFIC INFORMATION |
| Fentanyl | IM, IV, TM, | Parenteral preparation 100 times more |
| | IS | potent than morphine. Transmuscosal |
| | | preparation available for breakthrough |
| | | cancer pain. |
| Fentanyl | TS | Recommended for patients who have |
| (transdermal) | | continuous pain. Not suitable for rapid |
| | | titration due to slow onset and long |
| | | duration of action. (48-72 hrs) Do |
| | | not vut patch! |
| Levophanol | PO, IM, IV | Careful titration due to long |
| | | half-life of 12-15 hours. |
| Meperidine | PO, SQ, IM, | Should only be given immediately post |
| | IV, IS | procedures or surgery for a maximum of |
| | | 5-7 days. Not recommended for cancer |
| | | pain; active metabolite results in CNS |
| | | toxicity especially in patients with re- |
| | | duced renal function. Not suitable for |
| | | long-term use |
| Methdone | PO, IV, PR | Careful titration and monitoring due to |
| | | long pharmacologic half-life of 14-22 |
| | | hours but shorter analgesic effect of |
| | | only 6-8 hours. IV drug is VERY ex- |
| | | pensive and supply is limited. |
| Oxycodone | PO, PR | Consider low dose SR oxycodone in |
| | | patients requiring more than four doses |
| | | of short acting oxycodone per day. Use |
| | | caution when titrating products with |
| | | acetaminophen; hepatic toxicity has |
| | | been associated with acetaminophen |
| | | doses >4 grams/day. |
| Oxycodone (SR) | PO, PR | SR preparations are recommended for |
| | | ATC scheduled use only and should not |
| | | be crushed or broken (q 8-12 hr). |
| Codeine | PO, SQ, IM, | Use caution when titrating combination |
| | IV | products with acetaminophen. Hepatic |
| | | toxicity has been associated with |
| | | acetaminophen doses above 4000 mg |
| | | per day. |
| Hydrocodone | PO | Consider changing to a low dose SR |
| | | medication in patients requiring more |
| | | than six doses per day. Use caution |
| | | when titrating combination products |
| | | with acetaminophen. Haptic toxicity has |
| | | been associated with acetaminophen |
| | | doses above 4000 mg/day |
| Butorphanol | IM, IV, | Opioid agonist-antagonist analgesic. |
| | nasal | Do not administer with opioid. May |
| | | produce withdrawal in opioid dependent |
| | | patients. Not recommended for children. |
| Buprenorphine | IM, IV, IS | Opioid agonist-antagonist analgesic. |
| | | Do not administer with opioid. May |
| | | procedure withdrawal in opioid depend- |
| | | ent patients. |
| Nalbuphine | SQ, IM, IV | Decreases respiratory depression and |
| | | pruritis due to opioids while maintain- |
| | | ing analgesia. Opioid agonist-antagon- |
| | | ist. Do not administer with opioids. |
| | | May produce withdrawal in opioid de- |
| | | pendent patients. |
| Pentazocine | PO, SQ, IM, | Opioid agonist-antagonist analgesic. |
| | IV | Do not administer with opioids. May |
| | | produce withdrawal in opioid dependent |
| | | patients. Maximum oral dosage 600 mg/ |
| | | day. Max parental dosage 360 mg/day. |
| Tramadol | PO | Do not exceed 400 mg/day. De- |
| | | crease dose by 50% in renal impair- |
| | | ment. SR preparation currently under |
| | | investigation. |

PO '2 Oral
IM '2 Intramuscular
TD = Transdermal
PR = Rectal
SQ = Subcutaneous
IV = Intraveneous
TM = Transmuscosal
IS = Intraspinal
SR = Sustained Release
ATC = Around the Clock

Pain Crisis Intervention

When evaluating the patient's pain parameters and deciding a method of treating the pain, the etiology of the pain, the quantity of opioids consumed in the last twenty-four hours, and the presence of any dose limited side effects should be carefully considered.

The location to initiate treatment should depend on the patient's condition and home situation, whether or not there is a caregiver that can transport the patient to a clinic or hospital, and whether that caregiver is able to rapidly titrate oral opioids in the home. A major factor in deciding location is determined by whether the patient requires rapid intravenous treatment and titration, and if a nurse is available to perform home visitations.

Pain Crisis Intervention Questions?

What are the parameters of the pain to be treated?
Etiology of pain (new/old, known tumor sites).
Quantity of opiods consumed in last 24 hours.
Presence of any dose limiting side effects.
Where is the best location to initiate treatment?
Does the patient require rapid intravenous treatment and titration?
What is the patient's condition and home situation?
Is there a caregiver available to transport patient to clinic or hospital?
Presence of caregiver to rapidly titrate oral opioids in the home.
Availability of nurse to do a home visit.

Initiate the treatment with a fast acting opioid through whatever route is available. Give loading dose of immediate release opioid, equivalent to 25% of all opioids taken in the last 24 hours.

The clinician should reassess the patient's pain and the presence of side effects once an hour if the patient is taking medication orally, or once every fifteen minutes if they are receiving medication intravenously. The patient's level of sedation is especially important to pay attention to. If severe pain persists without any side effects, give 50% “rescue dose” of the previous 24 hour opioid dose, or the equianalgesic equivalent.

If severe pain is occurring with side effects, then the clinician should give a dose equivalent to 25% of the patient's previous 24 hour dosage. The side effects should be treated as soon as possible to maximize patient's comfort, and an intravenous method should be considered as a more direct route to relieving patient pain.

When the patient reports fair to significant relief through the use of oral medication, consider continuing the elevated dose of immediate release opioid for six to twelve hours, then re-titrate dose of an ATC opioid. If intravenous bolus injections were utilized, consider calculating amount of intravenous drug required to obtain patient relief and converting it to an oral dose utilizing the equianalgesic table. Increase the dose of ATC opioid to reflect the additional oral or intravenous opioid so the patient will be as well treated and comfortable as possible.

Rapid Opioid Titration Process

Give loading dose of immediate release opioid, equivalent to 25% of all opioids taken in the last 24 hours.

If pain crisis is co-existing with major side effect (i.e.: myoclonus or delirium) move rapidly to sequential opioid trials via parenteral or spinal route. Reassess pain and presence of side effects (primarily level of sedation) in one hour for oral or 15 minutes for intravenous.

If severe pain persists without side effects, give 50% of previous 24 hr opioid dose or equivalent at reassessment intervals as stated above.

If severe pain persists with side effects, repeat 25% of previous 24 hr opioid dose, consider starting IV, treat side effects at reassessment intervals as stated above. Patients with persistent pain with side effects should be considered candidates for hospitalization to stabilize.

When pain relief has been achieved:

If patient reports fair to significant relief with oral route of administration, consider continuing the elevated dose of immediate release opioid for 6-12 hours then re-titrate dose of ATC opioid.

If IV bolus injections were utilized, consider calculating amount of intravenous drug required to obtain relief and converting to oral dose.

Increase the dose of around the clock opioid to reflect the additional oral or intravenous opioid.

Sequential Opioid Trials

The ability to “switch” opioids is a critical process in tailoring analgesic therapy. Morphine has long been the standard first line drug in the opioid category. Today there is more a sense that all choices are equally important and can be utilized in virtually any order to achieve the best ratio of relief to side effects. There is no predicting which drug may be the “right” drug for a given patient. The key is to have a working knowledge of all the available choices and be prepared to try a third or fourth choice. Assure that the side effects are optimally treated whenever possible prior to switching drugs; this will avoid eliminating possible options before optimization has occurred.

EQUIANALGESIC CHART WITH 24 HOUR DOSING

| | Approximate | | | |
|---|---|---|---|---|
| | Equianalgesic | | | |
| Drug | Dose | | 24-hour dose | |
| Morphine | 30 | mg po q 4 h | 180 | mg |
| | 10 | mg IV q 4 h | 60 | mg |
| Morphine SR | 90 | mg q 12 h | 180 | mg |
| Hydromorphone (Dilaudid) | 7.5 | mg q 4 h | 45 | mg |
| Levorphanol (Levo-Dromoran) | 4 | mg q 6 h | 16 | mg |
| Methadone | 20 | mg q 6 h | 80 | mg |
| Oxycodone SR (Oxycontin) | 45 | mg q 12 h | 90 | mg |
| Duragesic (Fentanyl) Patch | 50 | mcg Δ q 72 hr | ≈180 | mg |
| Codeine | 180 | mg q 4 h | 1080 | mg |
| Hydrocodone | 30 | mg q 4 h | 180 | mg |
| Oxycodone | 30 | mg q 4 h | 180 | mg |

Dose equivalents for opioid näive adults/children ≧50 kg body weight

Example: Convert PRN Opioid to ATC

If 60 mg of immediate release morphine (4 doses of 15 mg MS IR) was used in the past twenty four hours, the dose is converted to 30 mg every twelve hours of sustained release morphine.

Note: Published tables vary in the suggested doses that are equianalgesic to morphine. Clinical response is the criterion that must be applied for each patient; titration to clinical responses is necessary. Because there is not complete cross-tolerance among these drugs, it is usually necessary to use a lower than equianalgesic dose when changing drugs and to re-titrate to response. (AHCPR Guidelines)

Drug Reduction Table

The Drug Reduction table is used to calculate the proper dosage of a medication when changing a patient from one opioid to another. When changing opioids select the current medication and dose from the “equianalgesic” column; move to the new drug and select the 24 hr starting dose from the “reduction” column.*

Drug Reduction Table

| | | | Use This Dose | |
|---|---|---|---|---|
| Low Dose | 24 hr Equianalgesic | | ˜30% Reduction | |
| MS | 30 | mg | 20 | mg |
| Codeine | 180 | mg | 120 | mg |
| Hydrocodone | 30 | mg | 20 | mg |
| Oxycodone | 30 | mg | 20 | mg |
| Hydromorphone | 7.5 | mg | 5 | mg |
| Levorphanol | 4 | mg | 2.5 | mg |
| Methadone | 20 | mg | 15 | mg |
| | | | Use This Dose | |
| Medium Dose | 24 hr Equianalgesic | | ˜30% Reduction | |
| MS | 180 | mg | 120 | mg |
| Duragesic | 100 | mcg/hour | 75 | mcg/hour |
| Oxycodone | 180 | mg | 120 | mg |
| Hydrocodone | 180 | mg | 120 | mg |
| Methadone | 120 | mg | 60 | mg (50%) |
| Hydromorphone | 40 | mg | 30 | mg |
| Levorphanol | 24 | mg | 16 | mg |
| Meperidine | 2400 | mg | 1680 | mg |
| | | | Use This Dose | |
| High Dose | 24 hr Equianalgesic | | ˜50% Reduction | |
| MS | 540 | mg | 270 | mg |
| Intravenous MS | 7.5 | mg/hr | 3.75 | mg/hr |
| Duragesic | 275 | mcg/hr | 150 | mcg/hr |
| Intravenous Fentanyl | 275 | mcg/hr | 150 | mcg/hr |
| Oxycodone | 540 | mg | 270 | mg |
| Methadone | 350 | mg | 180 | mg |
| Intravenous Methadone | 7.25 | mg/hr | 3.6 | mg/hr |
| Hydromorphone | 120 | mg | 60 | mg |
| Intravenous Hydromorphone | 1 | mg/hr | 0.5 | mg/hr |
| Levorphanol | 72 | mg | 36 | mg |
| Intravenous Levorphanol | 1.5 | mg/hr | 0.75 | mg/hr |
| Meperidine** | 7200 | mg** | 3600 | mg** |

**Meperidine is included in this table to facilitate changing patients to other more appropriate opioids. Meperidine is not safe for long-term use due to potential CNS toxicity.

Sample Case Studies: Equianalgesic Conversions

Case #2

Mr. Smith, a 65 y.o. man with prostate cancer, mets to bone, has been receiving good pain relief over the last month by taking Percocet 2 tablets q 4 h prn, and Salsalate 1500 mg q8 h. He usually takes about 2-3 doses of percocet a day. He calls, reporting that over the last several days he has needed to take more pain medicine. Yesterday he took a total of 9 doses of percocet (2 tablets each time). He is taking his salsalate regularly. The pain is in the usual sites he feels it, and it has not changed in character. He rates his worst pain at an 8/10, least 2/10, usual 5/10. A call is placed to the physician, and it is decided that he should switch to a sustained-release morphine tablet. But what dose?

QUESTIONS

1. PROBABLE ETIOLOGY OF PAIN (TUMOR-RELATED, TREATMENT-RELATED, NONMALIGNANT)

2. SITES

3. PAIN INTENSITY

4. PAIN CHARACTER (NOCICEPTIVE, NEUROPATHIC, MIXED)

5. WHAT BRINGS ON THE PAIN?

6. WHAT RELIEVES THE PAIN?

7. SIDE EFFECTS

9. DRUG DECISIONS

Case #2 (Continued)

1. Calculate the 24-hour total the patient used of the prn medication, then use the equianalgesic chart, determine the dosage of morphine needed.

1 Percocet=5 mg oxycodone, 325 mg Tylenol

2 Percocet=10 mg oxycodone, 650 mg Tylenol

Oxycodone: Morphine=1:1 (Oxycodone 30 mg=Morphine 30 mg PO)

10 mg×9 doses=90 mg in 24 hours.

The patient will need approximately 90 mg MS in 24 hours. Therefore the patient should receive MS SR 45 mg q 12 h or a combination to provide q 8 h dosing (30-30-30).

Other things to explore: Before making the switch to sustained-release morphine, it is a good idea to ask if there are times during the day when the pain is worse. Is the pain movement related? Does the pain wake him up at night? . . . This will help determine the best pain treatment plan for this patient.

2. Determine the appropriate breakthrough dosage.

10-30% of the 24 hour dose of scheduled medication is an adequate breakthrough dose.

The patient will require 9-28 mg of MS equivalent for breakthrough. At this point, percocet may still be adequate, but he may need closer to 20 mg per dose. Keep in mind the amount of Tylenol the patient will be taking with combination preparations. Consider an instant-release MS of 10 or 15 mg. The patient can easily take 2 MS IR's to give 20-30 mg of medication if that is what is needed to decrease his pain.

The patient should be instructed to report back within 24-48 hours regarding pain relief, or side effects. Also, he should report if he is continuing to take more than 3 breakthrough doses in a 24-hour period.

Long Acting Opioids: Transdermal Fentanyl

Overview

Note that the system (drug delivery) is long acting, not the drug. The transdermal fentanyl system represents a technological advance in pain management therapies. Originally thought of as an alternative to be used when the oral route became unavailable, many clinicians now agree that the benefits of fentanyl as a drug in the list of opioids to be used in sequential trials has surpassed the benefits of transdermal delivery alone. Fentanyl appears to be less constipating than other opioids. There may be a benefit for patient adherence in that subset of patients who report being “anti-pill”. Avoid the use of this dosage form in patients who have mostly episodic pain or current rapidly escalating pain.

Initiation

Opioid naive patients should be initiated with a 25 mcg/hr patch. Monitor efficacy and side effects as drug levels rise initially over 12-18 hours. Use equianalgesic dosing for opioid-tolerant patients. Previous meds should be continued for the first 18 hours. Avoid “adding” patches during the initial dose finding phase. Titrate unrelieved pain with short acting opioids and then increase the dose at the next regular 72 hour change.

Description

A potent synthetic mu opioid agonist. Specially designed transdermal system allows for continuous delivery over approximately 72 hours. Fentanyl is metabolized in the liver and contains no active metabolites.

Dosage and Administration

Educate patients to change the patch at about the same time of day every 3 days, to rotate the site, and to report any persistent skin irritation. Clip hair for patch, do not shave. Clean and dry skin; do not use soap, oil, lotion, or alcohol. Patches should not be cut in any way. Press firmly in place with palm of hand for 30 seconds. Titrate to efficacy. Utilize 24 hour total of short acting opioids as a guide for when to increase the patch size.

Ongoing Monitoring

Presence of fever increases absorption by up to 30%

Diaphoresis may make adhesion of the patch problematic.

Discontinue for persistent skin irritation with loss of integrity (blisters, lesions)

In general patients requiring greater than 300 μg should be converted to an alternate agent

Avoid multiple patches with differing replacement schedules.

Possible Side Effects

Sedation, Constipation, Nausea/Vomiting, Delirium, Myoclonus, Dry Mouth.

Precautions

Do not cut fentanyl patches. Particular caution should be used if WBC's or platelet counts are low in patients with skin irritation due to increased risk of infection and/or bleeding.

Long Acting Opioids: Oxycodone (OxyContin)

Overview

(Oxycodone is not a long acting drug, only through the drug delivery system OxyContin.) Long acting oxycodone formulation is widely used for constant pain of cancer and non-cancer sources. OxyContin comes in a range of dose strengths that are compatible with the vast majority of needed dosing configurations. This product has expanded our choices in the long acting category to further tailor therapy for the individual patient.

Initiation

Opioid naïve patients with constant pain can be safely started on the 10 mg dose, however it is more often the case that patients will be moving from a prn drug to long acting oxycodone via an equianalgesic conversion. Monitor during the first 3-7 days for side effects and titrate accordingly.

Description

Oxycodone is a mu opioid analgesic. The long acting formulation has a biphasic peak at 0.6 and 6.9 hours. Oxycodone is excreted primarily via the kidney.

Dosage and Administration

TABLETS ARE NOT TO BE BROKEN, CRUSHED OR CHEWED; the sustained release action of the drug is destroyed if the tablet's integrity is compromised; doing so will expose the patient to a rapid release of the full dose. Most patients can be maintained on an every 12 hour basis, although some may require every 8 hour dosing. While Purdue Frederick lists conversion from morphine to long acting oxycodone at 0.5:1; many expert clinicians believe the ratio is closer to 1:1 in cancer pain management. Consider increasing sustained release oxycodone in patients requiring more than four doses of short acting opioid per day.

Ongoing Monitoring

Assure optimized management of constipation

Titrate to efficacy

Possible Side Effects

Sedation, Constipation, Nausea/Vomiting, Delirium, Myoclonus, Dry Mouth.

Precautions

Any crushed or broken sustained release tablets should not be used.

Long Acting Opioids: Morphine (Sustained Release Preparations)

Overview

Sustained release morphine revolutionized the management of cancer pain twenty years ago. In particular the ability to provide effective pain relief throughout the night without having to awake at for taking a dose at was the beginning of the era of tailored analgesic therapy. It continues to be the mainstay of cancer pain management.

Initiation

TABLETS ARE NOT TO BE BROKEN, CRUSHED OR CHEWED. The sustained release action of the drug is destroyed if the tablet's integrity is compromised; doing so will expose the patient to a rapid release of the full dose. Sustained release morphine capsules (Faulding) can be opened and sprinkled onto apple sauce or used via PEG tubes as the sustained release action is in the granules. Sustained release morphine is generally utilized for opioid tolerant patients. Initiate by adding up the 24 hour total dose of current opioid and using the equianalgesic conversion chart to select a starting dose of sustained release morphine. Anticipate and educate patients regarding the expected side effects and monitor closely over the initial 3-7 days until steady state is achieved.

Description

The classic mu opioid agonist; only about 40% of orally administered morphine makes it to the CNS secondary to metabolism in the liver; the pharmacologic half-life is 2-4 hours while the duration of action is 8-12 hours; elimination occurs primarily as renal excretion of primary metabolites.

Dosage and Administration

Sustained release morphine can be dosed qd, q 12 h, or q8 h depending on the product and the individuals response to it. In general patients on q12 h or q8 h should have equal doses throughout the day but some patients do benefit from elevated doses during periods of highest pain (i.e. 60 mg at 9 PM and 90 mg at 9 AM to treat higher levels of day time pain). Titrate to pain relief or side effects according to pain intensity. Dosing q 8 hours may be beneficial if the patient is experiencing excessive sedation at q 12 hour dosing.

Ongoing Monitoring

Optimize management of constipation

Increase the dose of sustained release morphine if patient is requiring more than 4 doses of immediate release morphine per day

Strive for lowest frequency dosing schedule that works for the patient

Possible Side Effects

Sedation, Constipation, Nausea/Vomiting, Delirium, Myoclonus, Dry Mouth.

Precautions

Any crushed or broken sustained release tablets should not be used.

Long Acting Opioids: Levorphanol

Overview

One of many valuable “go to” opioids in the list of sequential trial options, levorphanol is listed as a long acting opioid but is considered by most clinicians more as a medium duration opioid due to it's six hour dosing interval. (Please note that a long half life is not to be confused with a drug's duration of action.)

Initiation

Levorphanol should generally be utilized in opioid tolerant patients. Because of its long half-life it should be initiated carefully and monitored for accumulation over the first 72 to 96 hrs. It is not generally a good choice for patients who are currently having escalating pain. If it is to be used in a relatively opioid naïve patient consider using low dose (1 mg bid or tid for the first few days until an initial efficacy versus toxicity evaluation can be done.

Description

Levorphanol is a synthetic mu agonist opioid analgesic. Levorphanol is available in one formulation—the 2 mg tablet. Levorphanol is 4 to 8 times as potent as morphine and has a long half-life (11-16 hrs) that can lead to accumulation of the drug. The drug is metabolized in the liver and excreted renally as an inactive metabolite. Because it is absorbed immediately from the gut it has the advantage over sustained release opioids in patients with rapid intestinal transit disorders where slow absorption formulations are not effective.

Dosage and Administration

Dose the patient every six hours with a combination of 2 mg or 1 mg (1/2) tab increments. In general avoid titrating the patient more frequently than every three days. If patients have significantly escalating pain treat with short acting opioids aggressively and then re-evaluate the dose of levorphanol once relief has been obtained. When doses are elevated patients may object to taking multiple pills. Many clinicians feel that despite the long half-life, clinical problems with levorphanol are fewer than with methadone.¹ ¹McCaffery M, Pasero C Clinical Manual of Pain. 1999

Ongoing Monitoring

Optimize treatment of constipation

If significant drowsiness occurs following an aggressive upward titration the next dose may need to be held, followed by a reduction in the dose and a slower titration schedule.

Possible Side Effects

Sedation, Constipation, Nausea/Vomiting, Delirium, Myoclonus, Dry Mouth.

Precautions

Avoid rapid titration due to long half life and plasma accumulation

Long Acting Opioids: Methadone

Overview

Methadone is listed here as a long acting opioid but is considered by most clinicians more as a medium duration opioid with it's 6 hour dosing interval. This drug carries some cultural baggage with it associated with narcotic detoxification as well as some clinical difficulties associated with its long half-life that makes it a less desirable option, particularly in the hands of the inexperienced provider. Make the distinction between elimination half life and duration of analgesic action. The analgesic action of methadone is shorter than elimination half life, which is not to be confused with methadone's duration of action.

Initiation

Methadone should generally be utilized in opioid tolerant patients. Because of its long half-life it should be initiated carefully and monitored for accumulation over the first 72 to 96 hrs. It is generally not a good choice in patients with rapidly escalating pain. If it is to be used in a relatively opioid naïve patient consider starting with 5 mg on a PRN basis, allowing the patient to initially determine the intervals (often two time a day dosing may occur in the beginning) and following up with dose finding based on individual response.

Description

Methadone is a synthetic mu agonist opioid analgesic. It is used widely in narcotic detoxification. The major clinically problematic feature of this drug is it's long half-life which has been reported to vary from 12 to 190 hours

Dosage and Administration

Careful titration and monitoring due to long half-life (in most people it is 14-22 hours), but the analgesic effect is 6-8 hrs. Clinicians have found that very close attention to titration is crucial because traditional conversion ratios underestimate the potency of methadone. Reassess pain and presence of side effects (primarily level of sedation) in one hour. If severe pain persists without side effects, give 50% of previous 24 hour period opioid dose or equianalgesic equivalent. If severe pain persists with side effects, repeat 25% of previous 24 hour opioid dose, consider starting intravenous infusion, treat side effects.

Ongoing Monitoring

When pain relief has been achieved, and patient reports fair to significant relief with oral route of administration, consider continuing the elevated dose of immediate release opioid for 6-12 hours then re-titrate dose of ATC opioid. Increase the dose of ATC opioid to reflect the additional opioid. Patients with severe pain with side effects should be considered candidates for hospitalization to stabilize. A short half-life opioid drug, such as fentanyl or morphine, is recommended for breakthrough pain when methadone is the mainstay analgesic.

Possible Side Effects

Sedation, Constipation, Nausea/Vomiting, Delirium, Myoclonus, Dry Mouth.

Precautions

Long half-life can lead to delayed toxicity from accumulation at start of therapy. Days or weeks of drug accumulation may occur after a period of rapid dose titration. Methadone is a poor choice for the elderly, noncompliant patients, or those with major organ failure.² ²Physician's Desk Reference. Medical Economics Company 1999

Short Acting Opioids: Codeine

Overview

Codeine is a short acting opioid considered as the “standard” in the weak opioid category. It is often utilized in acute postoperative and trauma states where only days to weeks of therapy are anticipated. It can be used chronically and when used in its single agent formulation does have the “no ceiling effect” properties associated with opioids. However there are three clinical issues that need to be raised when contemplating long term use:

Commonly used combination products (i.e. Tylenol #3) are unable to be titrated to high doses because of the hepatotoxicity of acetaminophen

Multiple expert clinicians have reported that codeine produces relatively more constipation than other opioids (not evidence-based)

There is currently no long acting formulation available

Initiation

Codeine, particularly in its 15 or 30 mg strength, is commonly started in opioid naïve patients without difficulty. Patients should be instructed that sedation, nausea, and constipation may occur with initiation, but sedation and nausea will likely be only transient. Patients should definitely be initiated on stool softeners immediately, roughly (100-250 mg) one for each 30-60 mgs per day of codeine and titrate to effect. Reassessment should be done at 3-5 days to check for persistence of sedation or nausea and for effectiveness of bowel regimen.

Description

A mu opioid agonist; plasma elimination half-life is 2.5 to 3 hours; mostly metabolized in the liver.

Dosage and Administration

Dosage should be titrated to adequate pain relief. Since codeine's effective duration of action is four hours, it should be dosed no longer than every four hours, administered around the clock for patients with continuous pain, or “as needed” for patients with episodic pain. Dosage and administration of combination products will be affected by the parameters of the 2^{nd} agent (i.e. no more than 13 Tylenol #4 tablets can be taken in a 24 hour period without reaching maximum dosage allowable for acetaminophen)

Ongoing Monitoring

Assure constipation is optimally treated

Monitor acetaminophen doses from combination products and any over the counter products containing acetaminophen

If patient is taking around the clock codeine products for persistent constant pain—convert to long acting opioid

Possible Side Effects

Sedation, Constipation, Nausea/Vomiting, Delirium, Myoclonus, Dry Mouth. Codeine has unpredictable absorption and a relatively high side effect rate profile.

Precautions

Use caution when titrating combination products with acetaminophen. Hepatic toxicity has been associated with acetaminophen doses above 4000 mg per day. Aspirin-containing products should not exceed 4000 mg/day.

Short Acting Opioids: Hydrocodone

Overview

Hydrocodone is a widely used short acting opioid that currently only comes as a combination product. It is commonly used for acute postoperative and trauma related pain of weeks to months duration. It is also widely used in the treatment of episodic chronic pain as a single agent or as a rescue drug in addition to a long acting opioid agent. It only comes as a combination product, making acetaminophen monitoring a must with this drug.

Initiation

Hydrocodone products, particularly in the 5 mg dosage formulations, are commonly started in opioid naïve patients without difficulty. Patients should be instructed that sedation, nausea, and constipation may occur with initiation, but sedation and nausea will likely be only transient. Patients should definitely be started on stool softeners immediately, roughly one (100-950 mg) for each 15-30 mgs per day of hydrocodone and titrate to effect. Reassessment should be done at 3-5 days to check for persistence of sedation or nausea and for effectiveness of bowel regimen.

Description

A mu opioid agonist; plasma elimination half-life is 4 hours; currently not available as a single agent formulation

Dosage and Administration

Dosage should be titrated to adequate pain relief. Available frequency of hydrocodone should generally be no greater than every four hours, although some patients may be able to dose every 6 hours. The drug should be administered around the clock for patients with continuous pain, or “as needed” for patients with episodic pain. Consider changing to a long acting medication in patients chronically requiring more than four doses per day (i.e. patients with constant pain every day). Combination products containing aspirin or ibuprofen are indicated for pain with an inflammatory component (bone pain, trauma pain, infection). Dosage and administration of combination products will be affected by the parameters of the 2^{nd} agent (i.e. no more than 5 of the hydrocodone preps that contain 750 mg of acetaminophen can be taken in a 24 hour period without reaching maximum dosage allowable for acetaminophen). Hepatic toxicity has been associated with acetaminophen doses above 4000 mg/day. Combination products containing aspirin should not exceed 4000 mg daily.

Ongoing Monitoring

Assure constipation is optimally treated

Monitor acetaminophen doses from combination products and any over the counter acetaminophen

If patient is taking around the clock hydrocodone products for persistent constant pain—consider converting to long acting opioid

Possible Side Effects

Sedation, Constipation, Nausea/Vomiting, Delirium, Myoclonus, Dry Mouth.

Precautions

Use caution when titrating combination products with acetaminophen. Hepatic toxicity has been associated with acetaminophen doses above 4000 mg per day.

Short Acting Opioids: Oxycodone

Overview

Arguably the most commonly used of all short acting opioid agents. It comes in a myriad of single and combination products. It is widely used for short term treatment of acute postoperative, trauma. or post-obstetrical pain. In addition it is used as a solo agent for patients with chronic episodic pain as a single agent or as a “rescue drug” for episodic pain in patients with underlying constant pain treated with long acting agents. Combination products must be dose limited at 4000 mg of acetaminophen or aspirin per day.

Initiation

Oxycodone products are commonly started in opioid naïve patients without difficulty. Patients should be instructed that sedation, nausea, and constipation may occur with initiation, but sedation and nausea will likely be only transient. Patients should definitely be started on stool softeners immediately, roughly one for each 15-30 mgs per day of oxycodone and titrate to effect. Reassessment should be done at 3-5 days to check for persistence of sedation or nausea and to verify effectiveness of bowel regimen.

Description

Oxycodone is a very commonly used mu opioid agonist. Oxycodone is only available as an oral drug.

Dosage and Administration

Dosage should be titrated to adequate pain relief. Oxycodone's duration of action necessitates no greater than every four hour administration, although some patients may be effectively dosed every 6 hours. The drug should be administered around the clock for patients with continuous pain, or “as needed” for patients with episodic pain. Consider changing to a long acting medication in patients chronically requiring more than four doses per day (i.e. patients with constant pain every day). Combination products containing aspirin are indicated for pain with an inflammatory component (bone pain, trauma pain, and infection). Rectal and parenteral formulations are not yet available in the United States, though oral preparations can be administered rectally. Dosage and administration of combination products will be affected by the parameters of the 2^{nd} agent (i.e. no more than 8 of the oxycodone preps that contain 500 mg of acetaminophen can be taken in a 24 hour period without reaching maximum dosage allowable for acetaminophen). Hepatic toxicity has been associated with acetaminophen doses above 4000 mg/day. Aspirin doses should not exceed 4000 mg/day.

Ongoing Monitoring

Assure constipation is optimally treated

Monitor acetaminophen doses from combination products and any over the counter preparations containing acetaminophen

If patient is taking around the clock oxycodone products for persistent constant pain—convert to long acting opioid

Possible Side Effects

Sedation, Constipation, Nausea/Vomiting, Delirium, Myoclonus, Dry Mouth.

Precautions

Use caution when titrating combination products with acetaminophen. Hepatic toxicity has been associated with acetaminophen doses above 4000 mg per day.

Short Acting Opioids: Hydromorphone

Overview

Hydromorphone is one of the “staples” of cancer pain management. Currently available as an immediate release short acting formulations, Many clinicians use it as the 2^{nd} line drug behind morphine for moderate to severe cancer pain. Definitely should be included in the realm of agents for sequential trials. May be considered as an acceptable alternative to morphine for the treatment of breakthrough or incident pain. Long acting formulation is currently not available.

Initiation

Hydrocodone products are generally used in opioid tolerant patients. This drug is an excellent choice for patients with severe episodic pain. Patients should be instructed that sedation, nausea, and constipation may occur with initiation, but sedation and nausea will likely be only transient. Patients should definitely be started on stool softeners immediately, roughly one for each 6-8 mgs per day of hydrocodone and titrate to effect. Reassessment should be done at 3-5 days to check for persistence of sedation or nausea and to verify effectiveness of bowel regimen.

Description

Hydromorphone is a mu agonist opioid analgesic; half-life is 2.5 to 3 hours.

Dosage and Administration

Dosage should be titrated to adequate pain relief. -Frequency of administration of hydromorphone should be no longer than every four hours. The drug should be administered around the clock for patients with continuous pain, or “as needed” for patients with episodic pain. Consider changing to a long acting medication in patients chronically requiring more than four doses per day (i.e. patients with constant pain every day). Because of its short duration, this may be a better drug for those with renal insufficiency, particularly the elderly. Oral controlled-release hydromorphone is available in Canada, and is currently not available in the United States

Ongoing Monitoring

Assure constipation is optimally treated

If patient is taking around the clock hydromorphone products for persistent constant pain—convert to long acting opioid

Possible Side Effects

Sedation, Constipation, Nausea/Vomiting, Delirium, Myoclonus, Dry Mouth.

Precautions

There may be a prolonged duration and cumulative effect in patients with hepatic or renal dysfunction.

Short Acting Opioids: Morphine Sulfate

Overview

Morphine is considered to be the standard first line drug in the opioid category. Immediate release morphine is widely used for the treatment of episodic pain with or without underlying constant pain in opioid tolerant patients. The drug comes in multiple dose sizes and multiple formulations including caps, tabs, elixirs, solutions, and suppositories. As with all opioids treatment includes the “titrate to effect or side effect” principle.

Initiation

Immediate release morphine is generally started in opioid tolerant patients that have “graduated” from weaker opioids, however if it is started in an opioid naïve patient initiate at the lowest dosage (i.e. 7.5-10 mg) titrated based on pain relief versus sedation. In the more likely scenario of initiation in the opioid tolerant patient one should consult an equianalgesic table to find the appropriate starting dose and then plan to reassess for efficacy versus toxicity over the initial 3-7 days.

Description

The classic mu opioid agonist; only about 40% of orally administered morphine makes it to the CNS secondary to metabolism in the liver; half-life is 2-4 hours; elimination occurs primarily as renal excretion of primary metabolites. Some metabolites are pharmacologically active and contribute to pain relief with long-term administration.

Dosage and Administration

Dosage should be titrated to adequate pain relief. Constipation must be prophylactically treated—approximately one stool softener (100-250 mg) for every 30 mg of morphine. Dosing frequency of immediate release morphine should be no longer than every four hours. The drug should be administered around the clock for patients with continuous pain, or “as needed” for patients with episodic pain. Consider changing to a long acting medication in patients chronically requiring more than four doses per day (i.e. patients with constant pain every day).

Ongoing Monitoring

Assure constipation is optimally treated

If patient is taking around the clock short acting morphine for persistent constant pain—convert to long acting opioid

If patient begins to have trouble swallowing assure that oral solution or rectal preps are readily available

Possible Side Effects

Sedation, Constipation, Nausea/Vomiting, Delirium, Myoclonus, Dry Mouth.

Precautions

In patients with reduced renal function. elimination of an active morphine metabolite occurs which can result in enhanced or prolonged opioid action.

Short Acting Opioids: Oral Transmucosal Fentanyl Citrate (OTFC

Overview

Oral transmucosal fentanyl is a fast-onset short-acting opioid in a “lozenge” formulation designed to be dissolved on contact with the buccal mucosa. The “Oralet” product was developed and is marketed for acute procedural related pain in opioid naïve patients and is limited to monitored setting. The “Actiq” product is FDA approved for management of moderate to severe pain in opioid tolerant patients. It is anticipated that this agent will be a major advantage in patients with severe episodic pain, particularly in those high risk patients who have very little constant pain between episodes making tailored opioid therapy very difficult.

Initiation

The Fentanyl Oralet preparation is designed for use in monitored settings where “conscious sedation” protocols are in place, primarily as a pre-med for procedures. Dose finding should be based on body weight. The Actiq preparation is initiated in opioid tolerant patients at the 200 μg dose over 15 minutes. A dose may be consumed 15 minutes after the previous unit is consumed. Use no more than two units per episode. Effective dosing achieved when adequate relief is achieved with a single OTFC unit. Its onset is very fast, within one to five minutes, but the duration is shorter (2-3 hours) by an hour than morphine.

Description

Transmucosal fentanyl is a potent synthetic mu opioid agonist. Onset of action for transmucosal fentanyl is similar to IV morphine bolus in clinical trials. Short duration of effect (i.e 1-2 hrs) makes is particularly appropriate for short duration pain episodes.

Dosage and Administration

Fentanyl Oralet is administered in a monitored setting, most commonly under a conscious sedation protocol in the opioid naïve patient. Actiq is titrated to the most efficacious dose and utilized prn. Most patients are managed with about 600 mcg. Daily doses above 6400 mcg have not been studied.

Ongoing Monitoring

Fentanyl Oralet administration is monitored according to institutional policy. Actiq in clinical trials required no special monitoring in opioid tolerant cancer patients. Cost of Actiq ranges between $7 and $17/dosage unit.

Possible Side Effects

Sedation, Constipation, Nausea/Vomiting, Delirium, Myoclonus, Dry Mouth.

Precautions

Dosage titration MUST be used to determine the correct dosage of Actiq. The correct dose is the dose where adequate relief is obtained from using one dosage unit. There is no correlation between the prior dose of opioid (in morphine equivalents) used for breakthrough pain and the final titrated dose of OTFC. Actiq units should be completely used and discarded according to directions. Store in a safe place away from children. Actiq is not to be used for acute post-operative pain or to be started in opioid naïve patients.

Non-Steroidal Anti-Inflammatories

Guiding Principles

NSAIDS are the co-analgesic of choice for pain from bone metastasis. They are often used in conjunction with opioids for treatment of mild to moderate cancer pain, as using an Opioid and an NSAID can provide the same analgesic effect as a higher dosage of opioids, with a greatly reduced risk of side-effects.

Dosing NSAIDS to full strength is recommended, as these drugs have a “ceiling” effect above which no further anti-inflammatory action is enhanced. NSAIDS can be given on an ATC basis; two weeks of treatment is usually required to assess for the full efficacy of the drugs.

For patients contraindicated to other NSAIDS, acetaminophen may be advantageous up to 4000 mg/daily, but be aware that cumulative dosing of combination products containing acetaminophen can be toxic. (4000+daily is toxic.)

NSAIDs are generally contraindicated in patients with peptic ulcer disease, significant renal impairment, thrombocytopenia, congestive heart failure, and for patients undergoing anticoagulant therapy. Elderly patients (>65 years of age) are generally initiated at lower doses. There are several agents that have profiles with less toxicity in some of these areas. Consult your pharmacist for drug selection on patients who could benefit from NSAID therapy but have potential risk.

NSAIDs DRUG CHOICES

| Common NSAIDs | Dose per day | |
|---|---|---|
| Ibuprofen | 2.4-3.2 | g |
| Ketoprofen | 550-1100 | mg |
| Choline magnesium trisalicylate | 1-4.5 | g |
| Diclofenac | 150-225 | mg |
| Salsalate | 4500 | mg |
| Flurbiprofen | 150-300 | mg |
| Nabumetone | 1.5-2 | g |
| Celebrex | 100-200 | mg po bid |
| Alternate NSAIDs | Dose per day | |
| Acetaminophen | 2000-4000 | mg |
| Combination products | Dose per day | |
| Diclofenac with misoprostol | 50-75 | mg with 200 mcg |
| Ibuprofen with hydrocodone | 200 | mg with 7.5 mg |

Frequently Asked Questions about Co-Analgesic Therapy

Q: Is it really necessary to use these medications when we can use opioids as a single agent and just give more?

A: In the pain algorithm study the use of co-analgesic therapies was consistently related to improved pain scores. In fact, in the patients of doctors and nurses that were trained on the algorithm the single most significant correlate to improved outcomes was the increased use of the neuropathic co-analgesics.

Q: You keep saying to optimize the NSAID drugs—aren't they dangerous?

A: The NSAIDs can be toxic, particularly in the elderly. Although the algorithm recommends optimizing NSAIDs, practitioners must monitor the patients continuously for signs of GI toxicity and renal sequelae. With monitoring these agents can be safely used. NSAIDs should be cautiously used in the elderly, particularly when cardiovascular and renal disease is present. In these situations co-administration clearly becomes less of a benefit.

Q: Are you saying that you should use both a tricyclic antidepressant and an anticonvulsant in neuropathic pain?

A: Using two drugs where one is effective is clearly not necessary if one works great! However we frequently will use a drug like gabapentin on top of low dose amitriptyline to get the best out of both drubs for patients with refractory neuropathic pain. These classes of drugs clearly have different sites of action and can work together to produce profound relief of neuropathic pain Both classes of drugs are worth a try, either separately or as combination therapy in the treatment of neuropathic pain.

Reassessment

Instruct patient to take NSAIDs with food.

Instruct patient to report any NSAID related GI distress.

Be aware that patient's often feel NSAIDs are “ineffective” because they do not control 100% of the pain—may need to reinforce the fact that these drugs assist the opioids and work in a different area of the pain cycle.

Be aware of renal toxicity associated with NSAIDs; semi-regular renal function tests (i.e.: Q 4-6 months) may be indicated based on patient condition.

When patients become thrombocytopenic from chemotherapy while on NSAIDs consider holding NSAIDs and/or changing to NSAID agents less associated with thrombocytopenia (i.e.: salsalate-check with your pharmacist)

Combination products containing NSAIDs are recently available. Check with your pharmacist regarding availability of dosing strengths and cost comparison.

Combination products containing misoprostol are contraindicated in pregnant women.

NSAID: Salsalate

Description

Salsalate is the ester of salicylic acid. Salsalate is completely absorbed in the GI tract, though the action occurs in the small intestine due to the drug's insolubility in gastric acid. This drug is often administered to patients that have adverse reactions to the GI distress caused by aspirin, or for patients that are at risk for bleeding from anticoagulation. Salsalate is similar to aspirin in effect, but it does not share the antipyretic or antiplatelet effects. This drug is good for rheumatoid arthritis and osteoarthritis as an analgesic. Salsalate is not appropriate for children. Each gram of salsalate provides approximately 1 gram of salicylate—the equivalent of 1.4 grams of aspirin.

Dosage and Administration

Salsalate peaks in two to four hours, and therapeutic levels may be maintained for up to sixteen hours with twice-a-day dosing. Recommended starting dose is 500-1000 mg bid. Maximum dose recommended is 4000 mg per day.

Initiation

Educate the patient that the NSAID may not relieve all the pain but still has significant co-analgesic effect particularly when added to the opioid; encourage the patient to continue it

Educate patient about the possibility of drug induced GI distress; the need to report it; the possible treatment with gastroprotective therapies; and the option of changing to a less irritating drug

Ongoing Monitoring

Continuously monitor for GI distress

Drug-Drug Interactions

Possible Side Effects

General: Nearly all of the NSAIDS can cause local irritation to the gastric mucosa as well as a systemic effect on prostaglandin synthesis throughout the body that manifests itself in the gut by interfering with the protective barrier in the GI mucosa. There are drugs that have more or less risk for damage to the mucosa and it appears to be dose related. In other words drugs that are low risk may become higher risk when utilized at high doses and drugs that are high risk may become lower risk at lower doses.

Salsalate Specific: Salsalate is one of the NSAIDs that is considered to have a lower risk for GI injury and minimal or no effect on platelets, though salicylates may raise plasma concentrations of salicyclic acid to toxic levels. The FDA and the American Academy of Pediatrics advise that salicylates not be used in children or teenagers with varicella or influenza unless directed by a physician.

Precautions

Salicylate use in children has not been established. The exact pathogenesis of Reye's syndrome and the potential role of salicylates in the pathogenesis remains to be determined.

NSAID: Celecoxib

Description

Celecoxib is a newly available NSAID that interferes with prostaglandin synthesis selectively through the cyclooxygenase-2 (Cox-2) enzyme system. The commonly used NSAIDs interfere with the Cox-1 system from which the GI toxicity effects are believed to occur. Celecoxib has anti-inflammatory, analgesic, and antipyretic qualities. This agent has demonstrated a significantly lower chance of GI distress when compared with naproxen or ibuprofen; however the evidence is less compelling in the studies comparing Celecoxib against diclofenac (see product insert). This agent should not be a first line choice (see NSAID related GI Distress protocol). Celecoxib has no to little effect on platelet aggregation and bleeding time. It is metabolized via the P450 system and plasma clearance could be impacted in poor metabolizers.

Dosage and Administration

Recommended oral dose is 200 mg per day, administered as a single dose or as two 100 mg doses twice per day. Absorption occurs approximately three hours after dosage is administered. Eating a high fat content meal before taking Celecoxib slows the absorption rate, but moderately raises the total absorption of the drug. Antacids cut down the absorption rate and efficacy of Celecoxib.

Initiation

Educate the patient that the NSAID may not relieve all the pain but still has significant co-analgesic effect particularly when added to the opioid; encourage the patient to continue it

Despite celecoxib's favorable profile patients should still be educated about the possibility of drug induced GI distress and the need to report it

Ongoing Monitoring

Continuously monitor for GI distress

In chronic therapy consider monitoring kidney function at least q 6 months, particularly in the elderly.

Drug-Drug Interactions

Drug interactions with cimetidine, co-trimoxazole, fluconazole, metronidazole, omeprazole, ACE inhibitors, furosemide, aspirin, fluconazole, lithium, methotrexate, and warfain may have drug-drug interactions with celecoxib.

Possible Side Effects

General: Nearly all of the NSAIDS can cause local irritation to the gastric mucosa as well as a systemic effect on prostaglandin synthesis throughout the body that manifests itself in the gut by interfering with the protective barrier in the GI mucosa. There are drugs that have more or less risk for damage to the mucosa and it appears to be dose related. In other words drugs that are low risk may become higher risk when utilized at high doses and drugs that are high risk may become lower risk at lower doses.

Celecoxib Specific: There is relatively little experience with the Cox-2 NSAIDs. From the clinical trials reported thus far in over 5000 patients treated up to 6 months the side effect profile appears to be very favorable for GI toxicity and without platelet effect. More experience with this agent is needed.

Precautions

Celecoxib is contraindicated in patients who have hypersensitivities to sulfonamides, or who have experienced asthma, urticaria, or other allergic type reactions from taking aspirin or other NSAIDS. Celecoxib should not be used in any patient with active ulcer disease. Although uncommon, NSAIDs can cause renal insufficiency and significant renal toxicity. Consider monitoring renal function every 6 months in patients on chronic therapy.

NSAID: Nabumetone

Description

Nabumetone is a prostaglandin synthesis inhibitor in the alkanone family. It has anti-inflammatory, analgesic, and antipyretic activity. It is a prodrug that must undergo liver metabolism to an active metabolite that is structurally similar to naproxen. It has a half life of 24 hours. The primary indication for nabumetone is the treatment of pain associated with osteoarthritis and rheumatoid arthritis in adult patients.

Dosage and Administration

Nabumetone has a longer half-life than most drugs in its category, thus dosing more frequently than once daily is unnecessary. Recommended starting dose is 1000 mg; maximum recommended dose is 2000 mg. Administering nabumetone with food increases the rate of absorption, but does not affect the extent of formation of active metabolite.

Initiation

Educate the patient that the NSAID may not relieve all the pain but still has significant co-analgesic effect particularly when added to the opioid; encourage the patient to continue it

Educate patient about the possibility of drug induced GI distress; the need to report it; the possible treatment with gastroprotective therapies

Ongoing Monitoring

Continuously monitor for GI distress

In chronic therapy consider monitoring kidney function at least q 6 months, particularly in the elderly.

Because nabumetone is a prodrug, metabolism to active metabolite may be impaired with severe hepatic impairment.

Drug-Drug Interactions

Warfarin may have drug-drug interactions with nabumetone. NSAIDs should not be administered concurrently with anticoagulants.

Possible Side Effects

General: Nearly all of the NSAIDS can cause local irritation to the gastric mucosa as well as a systemic effect on prostaglandin synthesis throughout the body that manifests itself in the gut by interfering with the protective barrier in the GI mucosa. There are drugs that have more or less risk for damage to the mucosa and it appears to be dose related. In other words drugs that are low risk may become higher risk when utilized at high doses and drugs that are high risk may become lower risk at lower doses. In addition this category of drugs generally interferes with platelet aggregation putting the patient at risk for an increased bleeding time.

Nabumetone Specific Side Effects

Nabumetone is one of the NSAIDs that is considered to have a lower risk for GI injury and minimal or no effect on platelets.

Precautions

Although uncommon, NSAIDs can cause renal insufficiency and significant renal toxicity. Consider monitoring renal function every 6 months in patients on chronic therapy.

NSAID: Choline Magnesium Trisalicylate

Description

Choline magnesium trisalicylate is a non-acetylated salicylate non-steroidal, antipyretic, and anti-inflammatory agent. Each gram contains 1000 mg of salicylate which is equivalent to 1.3 grams of aspirin. The drug is absorbed within one to two hours. It is primarily indicated for the treatment of osteoarthritis or rheumatoid arthritis.

Dosage and Administration

Choline magnesium trisalicylate is dosed twice daily. The starting dose is 500-1000 mg po bid with a maximum recommended dose of 4000 mg per day.

Initiation

Educate the patient that the NSAID may not relieve all the pain but still has significant co-analgesic effect particularly when added to the opioid; encourage the patient to continue it

Educate patient about the possibility of drug induced GI distress and the need to report it.

Ongoing Monitoring

Continuously monitor for GI distress

Monitor magnesium concentrations in patients with renal impairment.

Drug-Drug Interactions

Warfarin may have drug-drug interactions with choline magnesium trisalicylate.

Possible Side Effects

General: Nearly all of the NSAIDS can cause local irritation to the gastric mucosa as well as a systemic effect on prostaglandin synthesis throughout the body that manifests itself in the gut by interfering with the protective barrier in the GI mucosa. There are drugs that have more or less risk for damage to the mucosa and it appears to be dose related. In other words drugs that are low risk may become higher risk when utilized at high doses and drugs that are high risk may become lower risk at lower doses.

Choline Magnesium Trisalicylate Specific

Choline magnesium trisalicylate is one of the NSAIDs that is considered to have a lower risk for GI injury and minimal or no effect on platelets. Watch for hypermagnesemia especially in patients with renal impairment.

Precautions

This drug is not recommended for children or teenagers suffering from chicken pox, influenza, or flu symptoms, as it may be a catalyst for Reye's syndrome.

NSAID: Diclofenac

Description

Diclofenac is a potent prostaglandin synthesis inhibitor from the acetic acid family. The drug comes in immediate, enteric coated and extended release formulations and which results in differing absorption profiles. Diclofenac has anti-inflammatory, analgesic, and antipyretic activity.

Dosage and Administration

Starting dose of immediate release and enteric coated (delayed release) formulation is 25 mg po tid with a maximum recommended dose of 150 mg. The recommended dose of the extended release formulation is 100 mg qd.

Initiation

Educate the patient that the NSAID may not relieve all the pain but still has significant co-analgesic effect particularly when added to the opioid; encourage the patient to continue it

Educate patient about the possibility of drug induced GI distress and the need to report it.

Ongoing Monitoring

Continuously monitor for GI distress

Manufacturer recommends getting hepatic transaminase levels at 4 weeks from initiation and at intervals thereafter

In chronic therapy consider monitoring kidney function at least q 6 months, particularly in the elderly. (every 2-6 months)

Monitor for water retention especially in patients with heart failure or hypertension.

Drug-Drug Interactions

Aspirin, anticoagulants, digoxin, methotrexate, cyclosporine, lithium, oral hypoglycemics, and diuretics could have drug-drug interactions with diclofenac.

Possible Side Effects

General: Nearly all of the NSAIDS can cause local irritation to the gastric mucosa as well as a systemic effect on prostaglandin synthesis throughout the body that manifests itself in the gut by interfering with the protective barrier in the GI mucosa. There are drugs that have more or less risk for damage to the mucosa and it appears to be dose related. In other words drugs that are low risk may become higher risk when utilized at high doses and drugs that are high risk may become lower risk at lower doses. In addition this category of drugs generally interferes with platelet aggregation putting the patient at risk for an increased bleeding time. NSAIDs should not be administered concurrently with anticoagulants.

Diclofenac Specific: Diclofenac is one of the NSAIDs that is considered to have a lower risk for GI injury. Hepatic toxicity has been noted in 2% of patients on long term diclofenac therapy in a clinical trial of nearly 6000 patients. This drug may cause sodium and/or water retention. Significant drug interactions occur with thiazide diuretics, methotrexate, digoxin, and lithium salts.

Precautions

Hepatic toxicity has been reported with chronic diclofenac therapy. If clinical signs and symptoms consistent with liver disease develop and abnormal liver tests are detected, persist or worsen, discontinue diclofenac therapy. Although uncommon, NSAIDs can cause renal insufficiency and significant renal toxicity. Consider monitoring renal function every 6 months in patients on chronic therapy.

Flurbiprofen

Description

Flurbiprofen is a prostaglandin synthesis inhibitor from the propionic acid family (same family as ibuprofen). It is widely available in prescription and non-prescription doses. The half-life of flurbiprofen is 2-4 hrs. Flurbiprofen is similar to ibuprofen with some differences in peak, onset, and duration. While a patient may not find adequate relief from ibuprofen, they may gain relief from this drug.

Dosage and Administration

Starting dose is 100 mg po bid with a maximum dose of 300 mg per day. Plasma concentration is reached in 1 to 2 hours, and duration of analgesia is 4-8 hours.

Initiation

Educate the patient that the NSAID may not relieve all the pain but still has significant co-analgesic effect particularly when added to the opioid; encourage the patient to continue it.

Educate patient about the possibility of drug induced GI distress; the need to report it; the possible treatment with gastroprotective therapies.

Ongoing Monitoring

Continuously monitor for GI distress

In chronic therapy consider monitoring kidney function at least q 6 months, particularly in the elderly. Monitor for water retention especially in patients with heart failure or hypertension.

Drug-Drug Interactions

Flurbiprofen should not be administered with anti-coagulants.

Possible Side Effects

General: Nearly all of the NSAIDS can cause local irritation to the gastric mucosa as well as a systemic effect on prostaglandin synthesis throughout the body that manifests itself in the gut by interfering with the protective barrier in the GI mucosa. There are drugs that have more or less risk for damage to the mucosa and it appears to be dose related. In other words drugs that are low risk may become higher risk when utilized at high doses and drugs that are high risk may become lower risk at lower doses. In addition this category of drugs generally interferes with platelet aggregation putting the patient at risk for an increased bleeding time.

Flurbiprofen Specific: Some sources state that Flurbiprofen has less side effects than ibuprofen.

Precautions

Although uncommon, NSAIDs can cause renal insufficiency and significant renal toxicity. Consider monitoring renal function every 6 months in patients on chronic therapy. Toxicity can evolve in patients that have liver dysfunction, especially through continued use over long durations. Flurbiprofen treatment should cease 72 hours before adrenal function tests are to be administered.

NSAID: Ibuprofen

Description

Ibuprofen is the classic prostaglandin synthesis inhibitor in the propionic acid category. It has anti-inflammatory, analgesic, and antipyretic activity. It is widely available in prescription and non-prescription doses. The half-life of Ibuprofen is 2 to 4 hours. Ibuprofen is highly protein bound, metabolized in the liver. Urinary excretion of metabolites is complete within 24 hours.

Dosage and Administration

Recommended starting dose is 400 mg every 6 hours; maximum recommended dose is 3200 mg per day. Absorption is slowed if ibuprofen is taken with food, though the extent of absorption is not changed. Peak concentration is reached in 1 to 2 hours, analgesia in 2 to 4 hours, while a full anti-inflammatory effect takes a few days to 2 weeks.

Initiation

Educate the patient that the NSAID may not relieve all the pain but still has significant co-analgesic effect particularly when added to the opioid; encourage the patient to continue it

Educate patient about the possibility of drug induced GI distress; the need to report it; the possible treatment with gastroprotective therapies

Ongoing Monitoring

Continuously monitor for GI distress

In chronic therapy consider monitoring kidney function at least q 6 months, particularly in the elderly.

Ibuprofen treatment should cease 72 hours before adrenal function tests are to be administered.

Monitor for water retention especially in patients with heart failure or hypertension.

Drug-Drug Interactions

Ace inhibitors, anticholinergics. antidepressants, aspirin, CNS depressants, furosemide, lithium, methotrexate, and warfarin may have drug-drug interactions with Ibuprofen.

Possible Side Effects

General: Nearly all of the NSAIDS can cause local irritation to the gastric mucosa as well as a systemic effect on prostaglandin synthesis throughout the body that manifests itself in the gut by interfering with the protective barrier in the GI mucosa. There are drugs that have more or less risk for damage to the mucosa and it appears to be dose related. In other words drugs that are low risk may become higher risk when utilized at high doses and drugs that are high risk may become lower risk at lower doses. In addition this category of drugs generally interferes with platelet aggregation putting the patient at risk for an increased bleeding time. NSAIDs should not be administered concurrently with anticoagulants.

Ibuprofen Specific: Ibuprofen is frequently associated with gastropathies. Nausea, vomiting, diarrhea, constipation, flatulence, and abdominal pain are representative of the constellation of side effects attributed to ibuprofen. Ibuprofen reversibly inhibits platelet aggregation and patients should be monitored for easy bruisability, bleeding gums etc. The renal effects are from inhibition of renal prostaglandins resulting in diminished renal blood flow. Patients with conditions associated with diminished renal blood flow, such as CHF, cirrhosis, renal insufficiency, and advanced age are at highest risk for acute renal failure.

Precautions

Ibuprofen is contraindicated in patients with active or recent gastritis, peptic ulceration with or without perforation, GI bleeding, ulcerative colitis. Ibuprofen should be carefully administered to patients with hepatic, renal, or hemopoietic dysfunction, patients who routinely consume alcohol or smoke tobacco products (these behaviors are also conducive to ulcer development). The drug should only be administered with extreme caution to patients that are having anti-coagulation problems or therapy for anemia.

NSAID: Ketoprofen

Description

Ketoprofen is a prostaglandin synthesis inhibitor in the propionic acid category. It has anti-inflammatory, analgesic, and antipyretic activity. It is widely available in prescription and non-prescription doses. The half-life of Ketoprofen is 2 to 4 hours. Has been reportedly used rectally and compounded as a gel for topical administration. ketoprofen is similar to ibuprofen with some differences in peak, onset, and duration. While a patient may not find adequate relief from ibuprofen, they may gain relief from this drug.

Dosage and Administration

Recommended starting dose is 25 mg po q6-8 h; maximum recommended dose is 300 mg a day. Half life is 2-4 hours, duration is 4-8 hours. Patients with mild renal impairment should receive no more than 150 mg/day. Patients with estimated creatinine clearances <25 ml/min should receive maximum doses of 100 mg/day.

Initiation

Educate the patient that the NSAID may not relieve all the pain but still has significant co-analgesic effect particularly when added to the opioid; encourage the patient to continue it

Educate patient about the possibility of drug induced GI distress; the need to report it: the possible treatment with gastroprotective therapies

Ongoing Monitoring

Continuously monitor for GI distress

In chronic therapy consider monitoring kidney function at least q 6 months, particularly in the elderly. Monitor for water retention especially in patients with heart failure or hypetension.

Reduce dosage accordingly with reduced renal function.

Drug-Drug Interactions

Antacids, aspirin, diuretics, digoxin, warfarin, probenecid, methotrexate, and lithium may have drug-drug interactions with ketoprofen.

Possible Side Effects

General: Nearly all of the NSAIDS can cause local irritation to the gastric mucosa as well as a systemic effect on prostaglandin synthesis throughout the body that manifests itself in the gut by interfering with the protective barrier in the GI mucosa. There are drugs that have more or less risk for damage to the mucosa and it appears to be dose related. In other words drugs that are low risk may become higher risk when utilized at high doses and drugs that are high risk may become lower risk at lower doses. In addition this category of drugs generally interferes with platelet aggregation putting the patient at risk for an increased bleeding time. Studies to date are inconclusive concerning the relative risk of various NSAIDs. In general high dosages are associated with increased risk of serious effects. Increasing age, smoking, and alcoholism are highly associated with increased risk. NSAIDs should not be administered concurrently with anticoagulants.

Precautions

Although uncommon, NSAIDs can cause renal insufficiency and significant renal toxicity. Consider monitoring renal function every 6 months in patients on chronic therapy. Toxicity can evolve in patients that have liver dysfunction, especially through continued use over long durations.

NSAID: Diclofenac with Misoprostol

Description

Diclofenac is a potent prostaglandin synthesis inhibitor in the acetic acid family. Misoprostol is a synthetic agent used to prevent NSAID-induced ulcers in patients with a high risk of ulcers by reducing acid secretion during digestion and increasing bicarbonate and mucus production in the GI tract. The two combined make a potent drug combination usable by selected patients with GI disturbance. Diclofenac has a comparatively low GI toxicity profile to begin with and misoprostol adds protection. Misoprostol has been implicated in miscarriage and congential anomalies and therefore should not be administered to pregnant women or women seeking to become pregnant (see warning on Misoprostol primer). This should not be a first line drug (see NSAID related GI distress protocol).

Dosage and Administration

Starting dose is the 50 mg/200 mcg formulation bid and peak concentrations are achieved within 1-4 hours. Optimal dosing for mucosal protection would be the diclofenac 50 mg/200 mcg formulation given on a tid basis, resulting in 600 mcg per day. Dosing at 50-75 mg of diclofenac BID is equal to TID for pain control, but the combination with misoprostol at a BID dosing is less effective in preventing ulcers. The total misoprostol dose should not exceed 800 mcg/day and not more than 200 mcg/dose.

Initiation

Educate the patient that the NSAID may not relieve all the pain but still has significant co-analgesic effect particularly when added to the opioid; encourage the patient to continue it

Educate patient about the importance of staying with the tid dosing schedule to obtain the gastric mucosal protection.

Patients should be clearly warned regarding the potential for miscarriage or congenital abnormalities to themselves or to anyone they may divert the drug to; manufacturer recommends giving women of childbearing age a written warning

Ongoing Monitoring

Continuously monitor for GI distress and diarrhea

In chronic therapy consider monitoring kidney function at least q 6 months, particularly in the elderly.

Monitor hepatic transaminases within 4-8 weeks of starting therapy and periodically thereafter.

Drug-Drug Interactions

Anticoagulants, digoxin, metotrexate, cyclosporin, lithium, diuretics, aspirin, oral hyperglycemics, and antacids may have drug-drug interactions with diclofenac.

Possible Side Effects

Side effects reported with the combination product include abdominal pain, diarrhea, dyspepsia, nausea, and flatulence. For more information on each individual agent please see the primer on diclofenac and misoprostol respectively. Diarrhea may develop at the start of therapy and may last for up to 7 days.

Precautions

There are no additive effects that create new contraindications for the combination product—see Precautions for diclofenac and misoprostol respectively.

NSAID: Acetaminophen

Description

Acetaminophen is a para-aminophenol derivative that has fever-reducing and analgesic qualities. It is often used for patients that have a hypersensitivity to aspirin, or other NSAIDS, or have an intolerance to GI side effects of drug therapy in general. Patients taking acetaminophen also may benefit from this drug's lack of anticoagulation properties that are commonplace in aspirin and other NSAIDS. It is the safest antipyretic drug to use during pregnancy or lactation, but still should be administered cautiously. Acetaminophen is generally well tolerated by most patients.

Dosage and Administration

Acetaminophen is generally administered at 650-1000 mg po q4 h prn for episodic use or up to 650 mg po q4 h ATC. Acetaminophen is absorbed from the GI tract and/or the rectal mucosa. Peak concentrations occur within one hour. Half-life is 1 to 3.5 hours, and the duration of analgesia is 3 to 5 hours. It is metabolized in the liver and eliminated in the kidneys. No more than 4000 mg/daily of acetaminophen should be allowed to be administered to the patient for the danger of overdose and hepatic injury. Many OTC products contain small to medium amounts of acetaminophen, which if not checked could lead to accidental overdose.

Initiation

Counsel patient to read labels on any prescription and OTC meds they are taking to assure that the 4000 mg limit is not exceeded.

Educate the patient that the acetaminophen is unlikely to relieve all the pain but still has significant co-analgesic effect particularly when added to the opioid; encourage the patient to continue it

Ongoing Monitoring

Constant awareness of total daily dose of acetaminophen

Drug-Drug Interactions

CNS depressants and MAO man have drug-drug interactions with acetaminophen.

Possible Side Effects

Early signs of hepatic injury include anorexia, nausea, vomiting, pallor, and diaphoresis. Later signs include right upper quadrant pain, jaundice, elevated liver enzymes and a dramatic rise in prothrombin time. In rare extreme situations patients can experience acute renal failure, hepatotoxicity, and renal papillary necrosis, or renal tubular necrosis, anemia, leukopenia, thrombocytopenia, or pancytopenia.

Precautions

Acetaminophen overdose can be fatal; because acetaminophen depletes glutithione during metabolism only the administration of acetylcysteine (which is a source of glutithione) must be used as a potential antidote for overdose patients. Acetaminophen is contraindicated in patients with hepatic disease, viral hepatitis, or alcoholism, as the metabolism of the drug may be decreased and cause an increased risk of hepatotoxicity. Acetaminophen may have drug-drug interactions with phenytoin, barbiturates, carbamazepine, and ethanol.

Tricyclic and Other Analgesic Antidepressants

Guiding Principles

Tricyclic antidepressants (TCAs) are the co-analgesic of choice for patients with neuropathic pain, particularly those with a burning or numbing quality. A beginning dose of amitriptyline, nortriptyline, or desipramine is 10 mg for patients >70 years of age or patients who are “sensitive” to side effects. The average adult will have a beginning dose of 25 mg at hs.

“Start low and go slow” is the rule for titration. Titrate up by 10-25 mg every three to five days to the recommended dose range. Titrate up by 10-25 mg per day every three to five days to recommended dose range. Divided dose regimens (i.e.: 10 mg tid) as well as single dose evening (to minimize daytime sedation) regimens can be used.

TCAs have a “plasma therapeutic range” that can vary greatly from patient to patient (particularly in relation to drug-drug interactions). It is recommended that plasma levels be obtained at 75 mg per day of amitriptyline, nortriptyline, or desipramine and periodically to assess therapy. Blood level monitoring may be helpful in patients who show signs of toxicity, who are taking medications that affect anti-depressant metabolism, who show symptom breakthroughs following full response to medication, or who are taking more than 100 mg/day of amitriptyline, nortriptyline, or desipramine. Therapy should be periodically assessed to ensure the drug is right for the patient, as a full month trial period is usually needed to determine the efficacy of the drug, even though analgesic effect may occur with one to two weeks of initiation.

Amitriptyline has been the standard drug in this category, but is often associated with excessive sedation. (See Sedation Guiding Principles). It is a good choice for patients with difficulty sleeping. Nortriptyline generally has less sedation than amitriptyline, but desipramine is the most non-sedating of the tricyclics and is often the “go to” drug for patients with excessive sedation. For patients receiving concurrent treatment with anticonvulsants check with your pharmacist for potential drug-drug interactions before initiating therapy. Potential negative interactions can occur when combining certain medications from these two categories of drugs.

TCA DRUG CHOICES
Common Antidepressants

| | |
|---|---|
| Amitriptyline | 30-150 mg po qhs* |
| Nortriptyline | 30-150 mg po qhs* |
| Desipramine | 30-150 mg po qhs* |

Alternate Non-tricyclic Antidepressants

| | |
|---|---|
| Paroxetine | 20-50 mg po qam |
| Venlafaxine | 75-225 mg po qd* |

*Can also be prescribed in divided doses

Reassessment

Patients with pre-existing constipation may need increased bowel care (see constipation protocol)

Consider starting with or switching to desipramine in patients with pre-existing sleepiness; consider implementing excessive sedation protocol.

Ask about dry mouth—it is often overlooked and can be very distressing for patients (see dry mouth protocol).

Be aware of possibility of increased tricyclic serum levels in patients concurrently taking anticonvulsants or serotonin re-uptake inhibitors; doses may need to be adjusted downward.

Dysphoric side effects rarely resolve; therapy should be discontinued.

Titration of antidepressant therapy is usually done q 3-7 days. Consult pharmacist for individual drug recommendations.

When stopping antidepressants institute a tapering schedule of dose reduction every 3-5 days; abrupt discontinuation of antidepressants can cause significant negative somatic and psychic symptoms.

Tricyclic Antidepressants: Amitriptyline

Overview

Amitriptyline is a Tricyclic Antidepressant often used to treat depression. Amitriptyline is absorbed from the gastrointestinal tract. Amitriptyline is metabolized in the liver. The rate of metabolism varies from individual to individual depending on age, health, and other factors.

Initiation

Start at 10-25 mg daily (divided doses or at bedtime). Dosage of 75-150 mg have been used.

Description

Amitriptyline is an antidepressant with sedative effects. This drug can be used to provide analgesia without the side effects that would result from the use of opioids but careful titration is needed to ensure analgesic comfort. The drug is rapidly absorbed orally. The half-life of the drug is 10-50 hours. It is liver metabolized to an active metabolite nortriptyline.

Dosage and Administration

Dosage should begin at a low level and be carefully titrated upwards to match patient analgesia. Administration of the total dose at bedtime may minimize daytime sedation.

Ongoing Monitoring

Patients who exhibit photosensitivity should avoid sunlight. Anticholinergic side effects including: constipation, dry mouth, blurred vision, and urinary retention. Postural hypotension.

Drug-Drug Interactions

Cimetidine, epinephrine, MAO's, CNS depressants, and thyroid medications may have drug-drug interactions with amitriptyline.

Possible Side Effects

The adverse effects that can be caused with TCA's are quite extensive, including cardiovascular, anticholinergic, hematogic, and GI effects. The sedative effectives of amitriptyline are additive when combined with other CNS depressants. This drug can also cause constipation, urine retention, dry mouth, and blurred vision, a variety of anti-histamine effects, tremors, photosensitivity, cardiac arrhythmias, and sexual dysfunction may be caused by TCA's. Most of these effects will only manifest when the patient is receiving higher doses of amitriptyline.

Precautions

Amitriptyline should be used with caution for patients that have had a history of seizures, urinary retention, angle-closure glaucoma, or increase intraocular pressure. This drug is contraindicated for patients with cardiac disease. Arrhythmia, sinus tachycardia, and prolongation of the conduction time, and even cases of myocardial infarction and stroke can occur from use. Caution is advised with any drug-drug interactions involving TCA's and SSRI's, as Amitriptyline has a large number of drug-drug interactions with widely varying effects. Smoke, alcohol, and barbiturates will reduce the effectiveness of TCA's, while cimetidine can maintain serum levels longer than would be possible with the drug alone.

Tricyclic Antidepressants: Desipramine

Overview

Desipramine is a Tricyclic Antidepressant often used to treat depression. Despiramine is the active metabolite of imipramine. Desipramine is a good substitute for patients having untolerable side effects from other antidepressants. Desipramine is absorbed from the gastrointestinal tract. Desipramine is metabolized in the liver. The rate of metabolism varies from individual to individual depending on age, health, and other factors.

Initiation

Start at 10-25 mg daily usually at bedtime. Dosages of 75-150 mg may be needed. Titrate to pain relief.

Description

Desipramine is an antidepressant with sedative effects. Since opioids are not effective for neuropathic pain, desipramine can provide effective analgesia without opioid type side effects. Careful titration is needed to ensure analgesic comfort. Desipramine is well-absorbed from the gastrointestinal tract, and it has a half-life of 7-60 hours.

Dosage and Administration

Dosage should begin at a low level and be carefully titrated upwards to match patient analgesia. Administration of the entire dose at bedtime may minimize daytime sedation.

Drug-Drug Interactions

MAO, TCA's, SSRI's, tranquilizers, sedatives, and hypnotics may have drug-drug interactions with desipramine.

Ongoing Monitoring

Anticholinergic side effects including: constipation, dry mouth, blurred vision, and urinary retention. Postural hypotension.

Possible Side Effects

The adverse effects that can be caused with TCA's are quite extensive, including cardiovascular, anticholinergic, hematogic, and GI effects. The sedative effects of desipramine are additive with CNS depressants. This drug can also cause constipation, urine retention, dry mouth, and blurred vision, a variety of anti-histamine effects, tremors, photosensitivity, cardiac arrhythmias, and sexual dysfunction may be caused by TCA's. Most of these effects will only manifest when the patient is receiving higher doses of desipramine.

Precautions

Desipramine should be used with caution for patients that have had a history of seizures, urinary retention, angle-closure glaucoma, or increase intraocular pressure. This drug is contraindicated for patients with cardiac disease. Arrhythmia, sinus tachycardia, and prolongation of the conduction time, and even cases of myocardial infarction and stroke can occur from use. This drug is contraindicated for any patient that has recently undergone myocardial infarction or has cardiovascular disease, in patients with urinary retention or glaucoma, in patients that have thyroid disease or are taking thyroid medication, or in patients with a history of seizure disorder. Caution is advised with any drug-drug interactions involving TCA's and SSRI's, as Desipramine has a large number of drug-drug interactions with widely varying effects. Smoke, alcohol, and barbiturates will reduce the effectiveness of TCA's, while cimetidine can maintain serum levels longer than would be possible with the drug alone. This drug should not be administered with two weeks of the patient being treated with a MAO inhibitor drug as severe side effects or death could occur as result. Care should be made by the clinician to determine all of the factors that could lead to side effects or adverse side effects from desipramine.

Tricyclic Antidepressants: Nortriptyline

Overview

Nortriptyline is a Tricyclic Antidepressant often used to treat depression. Nortriptyline is the active metabolite of amitriptyline. Nortriptyline is absorbed from the gastrointestinal tract. Nortriptyline is metabolized in the liver. The rate of metabolism varies from individual to individual depending on age, health, and other factors.

Initiation

Initiate at 10-25 mg daily in divided doses or as single bedtime dose. Doses of 75-150 mg per day may be required with dosage titration.

Description

Nortriptyline is an antidepressant with sedative effects. This drug can be used for neuropathic pain since opioids are generally not effective for this type of pain. Careful titration is needed to ensure analgesic comfort.

Dosage and Administration

Dosage should begin at a low level and be carefully titrated upwards to match patient analgesia. Administration of the entire dose at bedtime may minimize daytime sedation.

Drug-Drug Interactions

MAO, dibenzazepines, TCA's, cimetidine, CNS depressants, quinidine, and SSRI inhibitors may have drug-drug interactions with nortriptyline.

Ongoing Monitoring

Anticholinergic side effects including: constipation, dry mouth, blurred vision, and urinary retention. Postural hypotension.

Possible Side Effects

The adverse effects that can be caused with TCA's are quite extensive, including cardiovascular, anticholinergic, hematogic, and GI effects. The sedative effects of nortriptyline are additive when combined with other CNS depressants. This drug can also cause constipation, urine retention, dry mouth, and blurred vision, a variety of anti-histamine effects, tremors, photosensitivity, cardiac arrhythmias, and sexual dysfunction may be caused by TCA's. Most of these effects will only manifest when the patient is receiving higher doses of nortriptyline.

Precautions

Nortriptyline should be used with caution for patients that have had a history of seizures, urinary retention, angle-closure glaucoma, or increase intraocular pressure. This drug is contraindicated for patients with cardiac disease. Arrhythmia, sinus tachycardia, and prolongation of the conduction time, and even cases of myocardial infarction and stroke can occur from use. This drug is contraindicated for any patient that has recently undergone myocardial infarction or has cardiovascular disease, in patients with urinary retention or glaucoma, in patients that have thyroid disease or are taking thyroid medication, or in patients with a history of seizure disorder. Caution is advised with any drug-drug interactions involving TCA's and SSRI's, as Nortriptyline has a large number of drug-drug interactions with widely varying effects. Smoke, alcohol, and barbiturates will reduce the effectiveness of TCA's, while cimetidine can maintain serum levels longer than would be possible with the drug alone. This drug should not be administered with two weeks of the patient being treated with a MAO inhibitor drug as severe side effects or death could occur as result. Care should be made by the clinician to determine all of the factors that could lead to side effects or adverse side effects from nortriptyline.

Non-Tricyclic Antidepressants: Paroxetine

Overview

Paroxetine is an SSRI antidepressant that effectively binds to plasma proteins. While the results will vary from patient to patient, a steady-state concentration should be reached within or about ten days. The half-life of this drug is about twenty one hours.

Initiation

Initiate at 10-20 mg daily in the morning with or without food (use lower dose in patients ≧65 years of age.

Description

Paroxetine has been proven more effective than other SSRI's for treating depression, and often is able to be used to treat patients that have resisted other anti-depression drugs. While paroxetine generates metabolites, the metabolites are of such a low strength and number that they do not threaten toxicity or other adverse effects.

Dosage and Administration

Because withdrawal effects may occur avoid abrupt discontinuance.

Drug-Drug Interactions

MAOI, Tryptopheine (?), Warfarin, and Sumatriptan may have drug-drug interactions with paroxetine.

Ongoing Monitoring

Monitor serum sodium in the elderly (for hyponatremia) receiving diuretics.

Possible Side Effects

A variety of side effects are possible depending on what paroxetine is being administered for. Common side effects include asthenia, sweating, nausea, dry mouth, decreased appetite, libido decreased, weight change, vital sign change, and other miscellaneous, non-life threatening side effects. Be sure to consult data on paroxetine side effects, as they are numerous and wide-spread across many physical and psychological areas.

Precautions

Elderly patients, or patients with hepatic or renal problems are likely to have increased plasma concentrations due to paroxetine, and there is the danger of toxicity from paroxetine build-up. Patients taking MAOI's should are contraindicated to take paroxetine could lead to serious adverse effects or even death. The clinician should carefully evaluate their patient's medical history before administering paroxetine. Paroxetine should not be used within fourteen days of the patient receiving MAOI drugs. This drug has a variety of drug-drug and should be researched carefully and administered accordingly. Treatment should not be discontinued immediately, but slowly titrated downward over the course of weeks. Paroxetine may inhibit TCA metabolism and cause adverse effects.

Non-Tricyclic Antidepressants: Venlafaxine

Overview

Venlafaxine is a phenylethylamine derivative that acts as a SSRI, but also blocks the reuptake of serotonin and norepinephrine with its ODV metabolite (O-desmethylvenlafaxine). It has weaker properties than other depressants as a dopamine reuptake inhibitor. Venlafaxine has a faster onset than most other antidepressants, and has a short half-life. Venlafaxine is absorbed and metabolized in the liver.

Initiation

Start at 25 mg three times daily and titrate upward to a maximum of 225 mg.

Description

Venlafaxine is effective for the treatment of depression.

Dosage and Administration

Take with food to decrease GI effects. Reduce dose 25% in patients with mild to moderate renal impairment. Reduce dose by 50% in patients undergoing hemodialysis or in patients with hepatic dysfunction. When the drug is discontinued, the dosage should be gradually decreased to reduce the risk of symptoms of discontinuation (dizziness, headache, nausea).

Drug-Drug Interactions

Anticoagulants, antigout drugs, antihypertensive medicine, diazoxide, pre-anesthetic, corticosteroids, cardiac glycosides, colestipol, hypoglycemics, lithium salts, methenamine, NSAIDS, norepinephrine, tubocuranine, phenytoin, phenobarbitol, cimetidine, and hepatic metabolitic drugs may have drug-drug interactions with venlafaxine.

Ongoing Monitoring

Renal function.

Possible Side Effects

A variety of side effects are possible depending on what venlafaxine is being administered for. Common side effects include asthenia, sweating, nausea, dry mouth, decreased appetite, libido decreased, weight change, vital sign change, and other miscellaneous, non-life threatening side effects. Be sure to consult data on venlafaxine side effects, as they are numerous and wide-spread across many physical and psychological areas.

Precautions

Elderly patients, or patients with hepatic or renal problems are likely to have increased plasma concentrations due to venlafaxine, and there is the danger of toxicity from venlafaxine build-up. Patients taking MAOI's should are contraindicated to take venlafaxine could lead to serious adverse effects or even death. The clinician should carefully evaluate their patient's medical history before administering venlafaxine. Venlafaxine should not be used within fourteen days of the patient receiving MAOI drugs. This drug has a variety of drug-drug and should be researched carefully and administered accordingly. Treatment should not be discontinued immediately, but slowly titrated downward over the course of weeks. Venlafaxine may inhibit TCA metabolism and cause adverse effects.

Anticonvulsants

Guiding Principles

Anticonvulsants are an important co-analgesic for patients with neuropathic pain, particularly those with a stabbing or “electric-like” component. While Anticonvulsants are very effective drugs, they are also frequently associated with sedation—and some patients may need lower doses than others to avoid this side effect. Sedation usually occurs at the initiation of therapy, and will resolve within days to weeks. An adequate trial period of one month is recommended to adequately test the efficacy of anticonvulsants, and therapy should be continued across pain intensity levels along with the use of opioids. Monthly serum levels are recommended (Agency for Health Care Policy Research). Serum levels are particularly important for patients on concurrent tricyclic antidepressants or with renal impairment. Less frequent levels may be adequate in stable patients. If a trial of clonazepam proves ineffective withdraw the drug slowly to avoid precipitating withdrawal symptoms.

When utilizing carbamazepine a baseline CBC with diff is recommended. Carbamazepine is initiated at 200 mg per day and titrated up by 200 mg every 3-4 days to max. of 800 mg/day. Clonazepam is initiated at 0.75 mg per day and titrated up to 1.5 mg in divided doses. Gabapentin can be initiated as low as 100 mg tid and slowly titrated up to 2500 mg tid or per day (or more). As carbamazepine has been associated with the development of reversible aplastic anemia, the clinician should re-check at 1 month and then approximately every 6 months.

For patients receiving concurrent treatment with antidepressants check with your pharmacist for potential drug-drug interactions before initiating therapy. Potential negative interactions can occur when combining certain medications from these two categories of drugs.

DRUG CHOICES
Common Anticonvulsants

| | |
|---|---|
| Gabapentin | 100-mg po tid to |
| | 600-mg po qid |
| | (note that much higher doses of Gabapentin |
| | have been reported anecdotally) |
| Carbamazepine | 100 mg po bid to 200 mg qid |
| Valproic Acid | 250-2000 mg q hs or in divided doses |

Alternate Anticonvulsants

| | |
|---|---|
| Lamotrigine | 25 mg po bid-150 mg po qid |
| Clonazepam | 0.25 mg tid-0.5 mg tid |
| Phenytoin | 300-500 mg po qhs |

Reassessment

If patients develops sleepiness or “spaciness” during taper up-slow down and plateau at the previous dose for twice as long-CNS effects do resolve in most patients.

When initiating carbamazepine therapy in a patient already on tricyclic antidepressants be aware of potential decreased blood levels of the tricyclic. Titrate TCA dose to response and adverse effects since concurrent carbamazepine use may require a higher tricyclic dose for response.

Gabapentin is almost totally cleared by the kidneys. Use with caution (at low doses) in patients with renal compromise.

Carbamazepine is associated rarely with aplastic anemia. Monitoring of CBC at least q 6 months is recommended.

Lamotrigine must be titrated slowly. Titrate up by 50 mg weekly or more slowly depending on side effect tolerance.

Anticonvulsants: Carbamazepine

Overview

Carbamazepine has been utilized in a wide variety of neuropathic pain states including trigeminal neuralgia, postherpetic neuralgia, diabetic neuropathy, phantom limb pain, and several others. The main areas of concern with carbamazepine in cancer patient management are 1) leukopenia/thrombocytopenia in approximately 2% of patients and rarely aplastic anemia and 2) drug interactions when co-administered with the tricyclics.

Description

Carbamazepine is an anticonvulsant in the iminostilbene derivative family. Its exact mechanism of action is unknown; although it is known to reduce polysynaptic responses much like phenytoin. It has some chemical similarities with the tricyclic antidepressants. It has an initial half-life of 25 to 65 hours. There is a wealth of data supporting its efficacy in trigeminal neuralgia. It reaches serum concentrations in a period of about four to five hours. With repeated dosing the half-life will decrease greatly down to a 25 to 65 hours, and with repeated dosing the half-life decreases to 12 to 17 hours due to the drug's ability to induce-metabolizing enzymes that initiate its own metabolism. It is absorbed from the GI tract and metabolized in the liver.

Initiation

A CBC should be obtained before the start of therapy. A leukocyte count less than 4000 is usually considered to be a contraindications to treatment.

Initiate at 100 mg bid—tid and plan to titrate up slowly

Caution the patient not to expect immediately analgesia; it may be 2-3 weeks before effect can be evaluated

Drug-Drug Interactions

Check to see if the patient is on any of the following drugs that can potentially interact with carbamazepine: cimetidine, danazol, diltiazem, isoniazid, macrolides (erythromycin), propxyphene, verapamil, antihistamines, barbiturates, primidone, charcoal, felbamate, hydantoins (phenytoin), serotonin selective reuptake inhibitors (SSRIs), theophylline, tricyclic antidepressants, valproic acid, fluoxetine, prophoxypene, valproate, riframpin, phenobarbitol, phenytoin, acetaminophen, anticoagulants, oral contraceptives, doxycycline, haloperidol, lithium, nondepolarizing neuromuscular blockers, oral diluents, antacids, posterior pituitary hormones, succinimide [bold print signifies other algorithm drugs or drugs likely to be present in the cancer population]—investigate drug-drug interactions as many times they will increase the level of carbamazepine and cause toxicity.

Dosage and Administration

Recommended dose range is 600 to 800 mg per day. Initiate at 100 mg twice daily. Sustained release formulation may improve patient adherence. Dosage should be taken with meals.

Ongoing Monitoring

A repeat CBC should be done after several weeks and then every 3to 4 months—a decline in leukocyte count to less than 3000 or an absolute neutrophil count of less than 1500 should trigger discontinuation of the drug. There is uncertainty as to whether periodic monitoring will signal the impending development of either thrombocytopenia or leukopenia.

If co-administered with tricyclics the carbamazepine levels can be expected to be high and the tricyclic levels can be expected to be low—checks levels and adjust accordingly.

Possible Side Effects

Most often reported side effect is drowsiness. Dizziness, vertigo, confusion, headache, visual hallucinations, tinnitus, and a variety of GI effects are possible: the list of side effects related to carbamazepine is quite extensive, covering more than fifty areas. Careful attention should be made by the clinician in regards to carbamazepine in drug-drug interactions, and especially in regards to the patient's condition and hypersensitivities. Additionally, photosensitivity can occur from taking this drug—appropriate sunscreen and clothing should be worn to reduce chances of sunburn.

Precautions

Carbamazepine is contraindicated for patients who are hypersensitive to tricyclic antidepressants, or with patients that have a history of bone marrow depression. Monoamine oxidase inhibitors should be discontinued at least two weeks before administering carbamazepine.

Anticonvulsants: Gabapentin

Overview

Gabapentin was first marketed as a uniquely differently anticonvulsant in 1994 and since that time has edged out carbamazepine as the most commonly prescribed anticonvulsant for neuropathic pain. Recently published data on its efficacy in diabetic neuropathy and post-herpetic neuralgia have underscored its efficacy. The comparative lack of drug interactions or serious toxicity when compared to carbamazepine has solidified its position as an adjuvant pain medicine.

Description

Gabapentin is an amino acid that is structurally related to the GABA neurotransmitter. It does not interact with GABA receptors nor is it metabolized to a GABA agonist or an inhibitor of GABA degradation. Unlike most anticonvulsants, it does not interact with neuronal sodium channels in the typical conduction blockade sense. Clinically, gabapentin fewer adverse side effects than other anticonvulsants and few problematic drug-drug interactions. Gabapentin can be added to drug regimens where there are already anti-epileptic drugs being used, without affecting the other agents levels in the patient. The drug is not appreciably metabolized, but excreted unchanged in the urine.

Initiation

Start at the 100 mg dose bid - tid and plan to titrate slowly

Caution the patient not to expect immediately analgesia; it may be 2-3 weeks before effect can be evaluated

Dosage and Administration

Target dose range is 1500-2500 mg per day. Higher doses have been reported. As dosages increase, the bioavailability of gabapentin decreases. Dosages administered over the recommended daily dose (300 mg-600 mg, three times daily) do not appear to cause significant adverse effects. Instruct the patient not to co-administer with an antacid as the antacid will reduce the bioavailability significantly. This drug should not be administered within two hours before or after an antacid is administered to the patient. Downward dose adjustments should be made in any patient with compromised renal function. Dosage should not exceed 600 mg/day for creatinine clearance of 30-60 ml/min and 300 mg/day for creatinine clearance of 15-30 ml/min; for creatinine clearance <15 ml/min, use 300 mg every other day.

Ongoing Monitoring

There have been no established recommended ranges of serum drug levels - titrate to effect.

The manufacturer recommends a significant dosing reduction based on creatinine clearance (see package insert or PDR)

Possible Side Effects

Drowsiness, fatigue, dizziness, and a sense of “disconnectedness” are often reported initially. These tend to subside with continue use. When withdrawing therapy, withdraw gradually over three to five days.

Precautions

Patients with a known hypersensitivity to gabapentin. Use with caution in patients with renal disease.

Anticonvulsants: Valproic Acid/Divalproex Sodium

Overview

Valproic Acid is an iminostilbene, anti-epileptic drug that is used to combat simple and complex absence seizures. Divalproex is a prodrug that dissociates into valproate in the gastrointestinal tract.

Initiation

Start at 125-250 mg two or three times daily and titrate to effective pain relief.

Description

Dosage and Administration

Administering this drug with food may minimize GI effects. Administration of valproic acid at bedtime can minimize some of sedation and dizziness effects. Patients unable to tolerate the gastrointestinal side effects of valproic acid may tolerate divalproex. When switching the same dose and schedule can be used.

Ongoing Monitoring

Blood levels should be monitored regularly for patients on a regime of valproic acid. Perform hepatic function tests before and every 2 months during the first 6 months then every 3-4 months. Discontinue immediately if hepatic function tests are elevated.

Platelet counts should be monitored regularly since thrombocytopenia incidence increases with total valproate serum levels above 110-135 mcg/ml.

Drug-Drug

CNS depressants, phenytoin, clonazepam, anticonvulsants, oral contraceptives,

Possible Side Effects

Adverse effects include standard GI effects: nausea, vomiting, indigestion, anorexia, abdominal cramps, and altered bowel condition. Other effects may include sedation, drowsiness, fatigue, ataxia, paresthesias, headache, nystagmus, anxiety, and behavioral disturbances. Valproic acid may alter blood, producing mild dyscrasias. Hemorrhage, bruising, or a coagulation disorder is a reason to reduce or stop therapy. Adverse endocrine effects may occur. Occasionally pruritus, photosensitivity, erythema, or muscle weakness can occur.³ ³Nursing Management in Drug Therapy, Lippincott, Williams & Wilkins 1999

Precautions

Valproic acid is contraindicated in patients with hepatic dysfunction.

Corticosteroids

Guiding Principles

Corticosteroids can be used when the patient presents with severe episodic pain due to nerve compression, or can be used cautiously in some chronic “pressure” related pain states. Initiate corticosteroid therapy with moderate to high dose per dosing guidelines. Efficacy of the drug should be apparent within 24 hours; taper to minimum dose required to maintain relief. Abrupt withdrawal of steroids may cause many serious side effects and can be fatal.

A patient is a candidate for steroid therapy if there is the presence of acute severe episodic pain (i.e. acute nerve compression), or if there is the presence of chronic “pressure” related pain states. Pressure related pain states include progressive visceral distention, severe lymphedema, increased intracranial pressure, soft tissue infiltration unrelieved by NSAIDS and opioids, and continuing nerve compression.

DRUG CHOICES

| | |
|---|---|
| For soft tissue infiltration: | Dexamethasone 2-4 mg po bid-tid |
| For nerve compression, visceral | Dexamethasone 4-8 mg po bid-tid |
| distention, lymphedema: | |
| For increase intracranial pressure: | Dexamethasone 4-12 mg po tid-qid |

EQUIVALENT DOSING

| | | |
|---|---|---|
| Methylprednisolone | 8 | mg= |
| Dexamethasone | 1.5 | mg= |
| Prednisone | 10 | mg |

Reassessment

If no relief in 24 hours, maximize dose per guidelines above.

Approximately 96 hours after effect achieved, consider gentle taper by lowering total daily dose 2-8 mg every 3 to 7 days.

Taper to minimum dose required to maintain relief.

Significant side effects may occur (i.e. hyperglycemia, infection, steroid myopathy/skin fragility, hypokalemia, steroid psychosis and emotional liability, GI erosions and perforation of the gut) but can be expected to decrease with dose taper.

Steroids: Dexamethasone

Overview

Dexamethasone is a glucocorticoid (adrenocortical steroid) related closely to prednisone. It is absorbed from the GI tract.

Initiation

Doses of 16-96 mg/day have been used in tumor-induced spinal-cord compression and reducing pain due to perineural edema and pressure on nerves.

Description

Dexamethasone is a long-acting steroid that can be used for replacement therapy in adrenocortical deficiency states. It is useful for treating altitude sickness, chemotherapy hyperemesis, bronchopulmonary dysplasia in preterm infants, bacterial meningitis, diagnoses of major depression, and hirsutism. Dexamethasone provides a range of effects including mood elevation, anti-inflammatory activity, anti-emetic activity, and appetite stimulation.

Dosage and Administration

Use the lowest possible dosage. Initiate at 4-8 mg three to four times daily. Higher doses have been used in pain associated with brain metastases and epidural spinal cord compression.

Ongoing Monitoring

Blood glucose and body weight.

Drug-Drug Interactions

Phenytoin, phenobarbitol, ephedrine, rifampin, and many other drugs may have drug-drug interactions with dexamethasone. Caution is advised.

Possible Side Effects

Long term use of this therapy may result in cataracts, glaucoma, diabetes, or ocular infections.

Precautions

Patients with systemic fungal infections are contraindicated. Corticosteroids may mask signs of infection, and new infections may develop during dexamethasone therapy. Patients that have spent time in the tropics, have diarrhea, or are suspected of amebiases should be ruled out for dexamethasone therapy due to the threat of activating latent amebiases. Dexamethasone may activate tuberculosis or tuberculin reactivity. Administration of live virus vaccines should not occur while a patient is taking corticosteroids, as these drugs suppress the immune system, making it more vulnerable to infections and diseases. Usage after a myocardial infarction is contraindicated due to an apparent association between the use of corticosteroids and the rupturing of the left ventricle free wall. In all these cases, great care must be maintained when planning a regime of drugs including dexamethasone.

Steroids: Prednisone

Overview

Prednisone is a glucocorticoid are used in replacement therapy in adrenocortical deficiency states, and has strong anti-inflammatory effects. There are three primary uses for prednisone:

Replacement therapy for adrenal sufficiency

Anti-inflammatory therapy for a wide variety of conditions

Immunosuppressant effects

Prednisone is absorbed from the GI tract. Prednisone is converted in the liver to prednisolone.

Initiation

Doses of 40-100 mg daily may be used for pain associated with brain metastases and epidural spinal cord compression. Steroids such as prednisone should be trialed for a period of 7 days. If there is any appreciable effect from one steroid, the dosage should be titrated downward to the point where the patient is gaining relief with the lowest possible dosages.

Description

While this is an effective drug for therapy, this drug compromises the immune system making the patient vulnerable to a variety of pathogens (infectious diseases). It affects almost every cell in the body, but not all in the same way. While it provides a variety of key anti-inflammatory effects, it does not affect antigen-antibody or immediate hypersensitive reactions.⁴ ⁴Nursing Management in Drug Therapy. Lippincott. Williams & Wilkins 1999

Drug-Drug Interactions

Phenytoin, phenobarbitol, ephedrine, rifampin, and many other drugs may have drug-drug interactions with prednisone. Caution is advised.

Dosage and Administration

Initiate at doses of 10-20 mg three to four times daily.

Ongoing Monitoring

The withdrawal of prednisone from a patient's regime should be done slowly and titrated carefully to avoid causing adverse effects. There are a variety of adverse drug-drug reactions including prednisone that the clinician should research before initiating therapy. Additionally, prednisone may produce a false-positive result in some tests for systemic bacterial infections. In addition, reactions to skin tests may be suppressed⁵ ⁵Nursing Management in Drug Therapy. Lippincott. Williams & Wilkins 1999

Blood glucose and body weight.

Possible Side Effects

Euphoria, headache, vertigo, nausea, vomiting, increased appetite, weight gain, and dyspepsia. Endocrine changes of menstrual irregularities, hyperglycemia, and suppression of pituitary ACTH release are common with continued prednisone use. Other side effects include muscle weakness, susceptibility to infection, sodium and fluid retention, masked signs of infection, fatigue, insomnia, and malaise.⁶ ⁶Nursing Management in Drug Therapy. Lippincott, Williams & Wilkins 1999

Precautions

The danger of the immune system being compromised during prednisone treatment is very high. Contraindications to prednisone use include hypersensitivity, systemic fungal infections, intramuscular use, and after the administration of live vaccines. Tartrazine or sulfites are often used in prednisone preparations, so individuals that have hypersensitive reactions to these substances should beware allergic or anaphylactic reactions. A long term effect of prednisone use is osteoporosis.

Bisphosphonates

Guiding Principles

Bisphosphonates are used for bone pain secondary to skeletal metastases. The efficacy of bisphosphonates usually becomes apparent one to two weeks after therapy initiation.

Pamidronate therapy may be repeated monthly. The safety and efficacy of more than two doses of intravenous etidronate has not been studied. Although efficacy has been demonstrated with oral etidronate, absorption is variable and GI side effects often result in discontinuation of treatment.

Oral etidronate therapy may be repeated 90 days after initial therapy discontinuation. Since etidronate inhibits bone mineralization, pamidronate may be preferred, especially since it can be repeated monthly.

A patient is suitable for bisphosphonate therapy if there is the presence of bone pain secondary to osteolytic bone destruction, if there is the presence of hypercalcemia or pathalogical fractures secondary to bone destruction, or the presence of uncontrolled opioid related side effects. These drugs may also be used if there is a desire for potential “opioid-sparing” effect.

The serum creatinine should be less than 2.5 mg/dl unless the benefits outweigh the risk of potential increased depression of renal function.

DRUG CHOICES
Pamidronate disodium

| | |
|---|---|
| 60-90 | mg IV monthly |

Etidronate disodium

| | |
|---|---|
| 7.5 | mg/kg/day IV for 3 consecutive days |
| 5-10 | mg/kg/day po qd or in divided doses for a maximum of 6 months |
| 11-20 | mg/kg/day po qd or in divided doses for a max of 3 months |

Reassessment

Monitor patients for potential reductions in serum calcium, phosphorus, potassium, and magnesium caused by bone regeneration. Maximum decreases in serum levels usually occur 72 hours after the first infusion.

If patient reports a decrease in pain after the procedure, opioids should not be stopped abruptly due to risk of withdrawal.

Downward titration: After pain relief is obtained, opioid doses may be decreased by 25-50% of the daily dose every one to two days until discontinuation or return of pain.

Bisphosphonates: Pamidronate Disodium

Overview

Pamidronate Disodium is a bisphosphonate that inhibits osteoclast activity, reduces bone re-absorption, and provides analgesia. This drug is often used to treat hypercalcemia.

Initiation

For patients with Paget's disease, recommended dose is 30 mg daily for three days. For patients with osteolytic lesions of multiple myeloma, a 90 mg dose on a four hour dose, once a month. For patients with osteolytic bone metastases of breast cancer, a 90 mg dose on a two hour schedule, once every two to three weeks. For moderate hypercalcemia is 60-90 mg. A 60 mg dose is given as a single dose intravenous infusion over at least four hours, while the 90 mg dose must be given by an initial single-dose, intravenous infusion over twenty four hours.

Description

Pamidronate disodium inhibits bone re-absorption without inhibiting bone formation or mineralization. These qualities combined with its analgesic abilities make for a drug that can be used to treat Paget's disease. osteolytic bone metastases of breast cancer, and the osteolytic lesions of multiple myeloma. Pamidronate is not metabolized and is exclusively eliminated through renal excretion.

Drug-Drug Interactions

Current research names no significant drug-drug interactions.

Dosage and Administration

60-90 mg IV daily. Adequate hydration and Pamidronate Disodium is indicated for moderate hypercalcemia associated with malignancy, with or without bone metastasis.

Ongoing Monitoring

Serum phosphate levels decrease after administration of Pamidronate disodium. Wait seven days between treatments, so patient will gain full effect from Pamedronate Disodium treatments.

Possible Side Effects

Elevation of temperature, soft-tissue symptoms at the site of the catheter insertion point. Fluid overload, generalized pain, hypertension, abdominal pain, anorexia, constipation, nausea, vomiting, urinary tract infection, bone pain, anemia, hypokalemia, hyomagnesemia, and hypophosphatemia are other kinds of adverse effects possible through the administration of this drug.

Precautions

Patients with anemia, luekopenia, and thrombocytopenia should be monitored carefully during the first two weeks of treatment. Serum calcium, electrolytes, phosphate, magnesium, and creatinine, and CBC, differential and hematocrit/hemoglobin must be carefully monitored. Pamedronate Disodium is contraindicated in patients that have a hypersensitivity to bisphosphonates. Metabolic parameters such as serum levels of calcium, phosphate, magnesium, and potassium should be carefully monitored following initiation of therapy. If hypercalcemia is brought on during treatment, short term calcium therapy. Overhydration should be avoided for patients with a danger of cardiac failure.

Side Effect Protocols: Constipation

Guiding Principles

All patients on opioids are to be initiated on a preventative bowel program due to the inhibition of peristalsis by opioids. Expect that all patients will experience constipation due to opioids. Clinicians should encourage fluids and a high fiber diet. Stool softeners or emollient laxatives should be used in combination with stimulant laxatives to ease defecation, though bulk laxatives are contraindicated for patients with poor intake or fluid intake of less than 1 liter per day. As the potential for constipation remains throughout opioid therapy, patients receiving higher doses of opioids will require higher doses or softeners/laxatives. Transdermal fentanyl is generally less constipating than other optoids.

For patients experiencing side effect constipation, a diagnostic workup of their bowel history, a digital exam, and a GI work-up is recommended.

Constipation Measures

| | |
|---|---|
| Patients with normal bowel | Increase fiber consumption and fluid |
| pattern/mild constipation: | intake |
| | Senna 1 tab PO qd - bid |
| | Docusate Capsule 250-500 mg |
| | qd - bid |
| | Milk of Magnesia 30 cc qd |
| For severe constipation: | Docusate Sodium 250 mg 1-3 caps |
| | up to qid |
| | Senna 1-3 tabs up to qid |
| | Bisacodyl 10-30 mg PO at hs |
| | Bisacodyl suppository 1-2 PR qd |
| For continuing or increasing | Add Lactulose 30-60 cc PO qd - tid |
| constipation: | Magnesium Citrate ½ to 1 bottle qd |

All patients on opioids require access to scheduled bowel medications, but some patients may find that prn dosing will suffice.

Reassessment

Reassess the patient's condition once or twice a week until they become regular. Instruct the patient to call if they are unable to maintain bowel adequate movement at least q 3 days. The need to assess the amount of stool the patient is having is often overlooked by clinicians. Patients using over the counter laxatives need to be assessed for amount and variety of medication ingested due to a greater chance of taking medication inappropriately or incorrectly. Cautious use of magnesium containing products in patients with renal insufficiency.

Bisacodyl

Overview

Bisacodyl is a laxative that has a direct effect on the intestinal mucosa and readily stimulates peristalsis. This drug is actuated in the colon and has tendencies similar to that of magnesium hydroxide.

Description

Bisacodyl induces laxative qualities by pulling water into the colon. It also stimulates sensory nerve endings to produce parasympathetic reflexes resulting in peristalsis of the colon.

Dosage and Administration

10-30 mg po at hs, or for the bisacodyl suppository 1-2 PR qd. Bowel movements happen approximately six hours after oral administration, and within an hour of rectal administration. The product should not be used for more than seven days at a time. Results will take place within fifteen minutes to an hour.

Ongoing Monitoring

Possible Side Effects

Rectal bleeding, blistering, burning, or itching.

Precautions

These laxatives are contraindicated for patients with acute surgical abdomen, appendicitis, rectal bleeding, gastroenteritis, or intestinal obstruction. Do not use bisacodyl when nausea, abdominal pain, or vomiting is present.

Docusate Sodium

Overview

Docusate Sodium is a stool softener. Stool softeners (emollient laxatives—e.g., docusate) encourage bowel movements by helping liquids mix into the stool and prevent dry, hard stool masses.

Dosage and Administration

250 mg 1-3 caps up to qid. Useful to administer for patients suffering constipation when other peristaltic drugs are contraindicated, or constipation is a risk after surgery. Patients that are contraindicated to strain at stool, such as after myocardial infarction, eye surgery, or anorectal surgery may benefit from docusate sodium.⁷ Results will take place from two to fifteen minutes from insertion. ⁷Nursing Management in Drug Therapy, Lippincott, Williams & Wilkins 1999

Possible Side Effects

Rectal bleeding, blistering, burning, or itching. Bitter taste, throat irritation, and nausea are very remote side effects.

Precautions

These laxatives are contraindicated for patients with acute surgical abdomen, appendicitis, rectal bleeding, gastroenteritis, or intestinal obstruction. Do not use bisacodyl when nausea, abdominal pain, or vomiting is present.

Senna

Overview

Senna is a laxative that has a direct effect on the intestinal mucosa and readily stimulates peristalsis. This drug is actuated in the colon and has tendencies similar to that of magnesium hydroxide.

Description

Senna induces laxative qualities by pulling water into the colon. It also stimulates sensory nerve endings to produce parasympathetic reflexes resulting in peristalsis of the colon.

Dosage and Administration

1-3 tabs up to qid. Bowel movements happen approximately six hours after oral administration, and within an hour of rectal administration. Senna should not generally be used for more than seven days. Results will take place in anywhere from thirty minutes to two hours.

Ongoing Monitoring

If there is a noticable chance in bovyel movements that last longer than two weeks, or the change keeps re-occurring, check with your doctor before you use a laxative.

Possible Side Effects

Rectal bleeding, blistering, burning, or itching.

Precautions

These laxatives are contraindicated for patients with acute surgical abdomen, appendicitis, rectal bleeding, gastroenteritis, or intestinal obstruction. Do not use Senna when nausea, abdominal pain, or vomiting is present.

Side Effect Protocols: Nausea

Guiding Principles

Transient nausea at the onset of opioids, or with aggressive titration is common; “tolerance” to this side effect usually develops within 72 hours. If nausea/vomiting is occurring at the initiation of an opioid consider prophylactic course of antiemetic therapy for 72 hours.

Many patients prefer pain instead of nausea. Patients with nausea are at high-risk for non-adherence to opioid therapy. Order prophylactic antiemetics for patients with a history of opioid-induced nausea. A change in opioid should be considered in any patient with opioid-induced nausea that persists for more than 72 hours, despite antiemetic treatment. Patients with a history of nausea sensitivity to medication may require changing medication more frequently than 72 hours to avoid non-adherence.

Polypharmacy often confuses the etiology of nausea; attempt to make changes one step at a time to clarify causation. For patients experiencing nausea, an evaluation for constipation should be performed. Whether there is evidence of other drug etiology or bowel or other gastric outlet obstruction may be required. Checking for drug-drug interactions and electrolyte, liver, and kidney function tests may be necessary. If the patient has a history of nausea or vomiting with opioids previously, consider prophylactic therapy for 72 hours.

Drugs used to treat nausea

| Drug | Dosage and Schedule |
|---|---|
| Metoclopramide | 5-10 mg PO/IV qid |
| Prochlorperazine | 5-10 mg PO/IV or 25 mg PR q 4-6 hrs or |
| | sustained release 10-15 mg PO q 12 hrs |
| Transdermal scopolamine | 1.5 mg (one patch topically) q 72 hrs |
| Haloperidol | 0.5-2 mg PO q 4-6 hrs |
| Diphenhydramine | 25-50 mg PO/IV q 4-6 hrs |
| Hydroxyzine | 25-100 mg PO q 6-8 hrs |
| Thiethylperazine | 10 mg PO q 8-12 hrs |
| Lorazepam | 0.5-1 mg PO q 4-6 hrs |
| Ondansetron | PO q 8-12 hrs (serotonin antagonist should be |
| | considered only if dopaminergic antagonists |
| | have failed) |

Note
Antihistamines probably will not be as effective as antiemetics since they lack dopamineric effects. Consider adding droperidol 0.625-1.25 mg IV/IM q 4 hrs. All of the above can be either PRN or ATC.

Reassessment

Within 24 to 48 hours a reassessment of the nauseated or vomiting patient should be performed. Nausea may subside within 72 hours of the initiation of treatment; if it does decrease evaluate the patients condition two to three times per week until the side effects resolve.

| | |
|---|---|
| Suspect opioid-induced | Initiate single or combination antiemetics. |
| nausea/vomiting | Maximize co-analgesics for “opioid sparing” |
| | effect and trial decreased opioid if pain |
| | control adequate. |
| Suspect other etiology for | Hold/change suspect agent if possible. |
| nausea/vomiting | |

Continue scheduled antiemetic(s) if tolerated. Reassess at 48-72 hours. If nausea/vomiting persists and suspect opioid etiology, it is recommended to review the patient's drug history. To perform sequential opioid trials, perform equianalgesic conversion to the next opioid being administered (see table below), and reassess the effectiveness of the new opioid within the next twenty four to forty eight hours. Repeat with other opioids if symptoms persist. If Nausea and/or Vomiting persists, consider Inpatient or Ambulatory Evaluation, and evaluate for regional anesthetic techniques for opioid sparing effects.

Metoclopramide

Overview

Metoclopramide is a GI stimulant that increases the effect of acetylcholine within the GI system, allowing normal GI function to occur. It is absorbed from the GI tract.

Description

Metoclopramide is often used to treat nausea and vomiting in post-operative patients, and may relieve symptoms of diabetic gastroparesis and GERD The drug stimulates motility in the upper GI tract, but does not stimulate secretions associated with the GI tract.

Drug-Drug Interaction

Patients who are receiving EPS drugs should not take metoclopramide concurrently. Levodopa and metoclopramide have opposite effects on dopamine receptors. Anticholinergics and narcotics antagonize the GI motility effects from metoclopramide. Careful research is recommended before the clinician administers this drug.

Dosage and Administration

10 mg, po qid

Ongoing Monitoring

Monitor carefully for any side effects. and adjust treatment accordingly.

Possible Side Effects

Restlessness, drowsiness, insomnia, fatigue, and other CNS related effects are likely, as well as nausea, diarrhea, and transient hypertension. Mental depression with suicidal ideation is possible even for patients that have not suffered clinical depression before. Adverse effects include EPS symptoms, tardive dyskinesia, akathisia, and Parkinsonism-like reactions are possible.

Precautions

Metoclopramide is contraindicated for patients that GI motility would be dangerous, and for patients with seizure disorders, Parkinson's disease, pheochromocytoma, and a history of breast cancer. Patients who are receiving medications to treat EPS induced by drugs should not take metoclopramide concurrently. It may be given to patients diagnosed with depression only if the benefits outweigh the potential risks. Levodopa and metoclopramide have opposite effects on dopamine receptors. Anticholinergics and narcotics antagonize the GI motility effects from metoclopramide. Careful research is recommended before the clinician administers this drug.

Prochlorperazine

Overview

Prochlorperazine is a phenothiazine that has significant antiemetic effects and a high incidence of extra pyramidal systems.

Description

Prochlorperazine is often used to treat post-operative nausea and vomiting, chemotherapy and radiation sickness. It can also be used to treat the manifestations of psychotic disorders. It is effective for the treatment of short-term anxiety, but is not often the key drug chosen for such treatment due to its risk factors—such as the possibility of developing tardive dyskinesia.

Drug-Drug Interactions

For patients being treated for cancer, the nausea-reducing tendencies of prochlorperazine may prevent the patient from vomiting, one of the usual signs of patient toxicity from cancer-related drugs. Careful research is required before administering this drug to a patient due to the wide number of adverse effects possible from taking prochlorperazine.

Dosage and Administration

10-15 mg po/IV or 25 mg PR q 4-6 hrs. Sustained release 10-15 mg po q 12 hrs.

Ongoing Monitoring

Monitor carefully for any side effects, and adjust treatment accordingly.

Possible Side Effects

Hypotension, deep sleep, and coma states have occurred.

Precautions

The antiemetic tendencies of this drug may mask signs of overdosage from other drugs and may make the diagnosis and treatment of intestinal obstruction, brain tumors, and Reye's syndrome difficult.

Oversedation

Guiding Principles

Sedation may be caused by opioid, antidepressant, or anticonvulsant medications used alone or in combination. Transient sedation at the onset of opioids is common and “tolerance” to this side effect usually develops within 72 hours. It is important to reassure patient/family that this early sedation is expected and is usually transient.

If sedation is occurring at the initiation of an opioid wait 72 hours prior to adding stimulants or changing the pharmacologic agents. Sedation occurring at the initiation of antidepressant or anticonvulsant therapy is also common but tolerance will take longer to develop. This side effect can be minimized by starting with lower doses and titrating slowly to the proper effect. The patient may benefit from additional sleep if pain has been relieved for a significant period of time.

If undesirable sedation persists for more than seven days, an alteration in the analgesic approach is required. Treatment of drug induced sedation should be tried before progressing to any invasive therapy. When assessing oversedation in a patient, check electrolyte levels and perform liver & kidney function tests. Check for concurrent drug interactions, and research any CNS involvement.

Drugs used to treat sedation

| Drug | Dose and schedule | |
|---|---|---|
| Caffeinated beverages | Scheduled ingestion | |
| Anhydrous Caffeine | 100-200 | mg PO bid - tid prn |
| Pemoline | 37.5-112.5 | mg PO qd |
| Methylphenidate | 2.5-10 | mg at 8a and 2p |
| Dextroamphetamine | 5-10 | mg at 8a and 2p |

If the patient finds the sedation “excessive”, contact with the patient should be made once every twenty four to seventy two hours until the sedation problem is resolved. Non-terminal unresponsive patients require an emergency response (naloxone, and calling 911), while terminal patients may find sedation desirable. If sedation occurs at initiation of opioid Rx or with aggressive titration, be sure to reassure patient that “tolerance” to sedation commonly occurs after 72 hours on a stable dose. If at all possible allow 72 hours to pass prior to initiating treatment for undesirable sedation. Reassess situation every 48 to 72 hours.

Sedation reassessment

| | |
|---|---|
| Suspect Opioid-induced | Maximize co-analgesics for opioid sparing |
| sedation | effect and trial decreased opioid if pain |
| | control adequate |
| Suspect TCA-induced | Dose at bedtime (if not currently) |
| sedation | Switch to secondary amine TCA |
| | Change from Amitriptyline to Nortriptyline |
| | Change from Nortriptyline to Desipramine |

If undesirable sedation persists. review the patient's drug history, and consult the equianalgesic conversion tables for options on other opiods to administer instead of the current medication. Reassess any new opioids every twenty four to forty eight hours.

If undesirable sedation still persists, institute stimulant therapy. If aggressive treatment is desired consider Inpatient or Ambulatory Care Evaluation. Evaluate for regional anesthetic techniques for opioid sparing effect.

Reassessment

Non-terminal unresponsive patients require an emergency response; the administration of naloxone may be necessary, as calling 911 for aid. If a patient believes that the sedation side effects is excessive, monitoring the patient once every one to three days is required until the sedation problem is resolved. Keep in mind that terminal patients may find sedation desirable.

| Suspect Opioid-induced | |
|---|---|
| sedation | Suspect TCA-induced sedation |
| Maximize co-analgesics | Dose at bedtime (if not currently) |
| for opioid sparing effect | Switch to secondary amine TCA |
| and trial decreased opioid | Amitriptyline → Nortriptyline → Desipramine |
| if pain control adequate | |

Caffeine

Overview

Caffeine is a xanthine CNS stimulant that can be found in many products including chocolate, sot drinks, coffee and tea, and analgesics and weight control drugs. It is rapidly absorbed and distributed throughout all of a patient's body tissues and fluids. Half-life is about three hours.

Description

Caffeine increases heart rate and blood pressure in the patient, causes alertness, lessens fatigue, increase the patient's ability to concentrate, and impart a mild euphoric feeling.

Dosage and Administration

Caffeinated beverages—scheduled ingestion. Anhydrous caffeine may be given in 100-200 mg po bid - tid prn.

Possible Side Effects

Insomnia, hypertension, and cardiovascular problems can occur if this drug is used too frequently or in too large doses. Excess amounts can also cause insomnia, irritability, and exhaustion along with a variety of other mood and energy altering effects.

Precautions

These laxatives are contraindicated for patients with acute surgical abdomen, appendicitis, rectal bleeding, gastroenteritis, or intestinal obstruction. Do not use senna when nausea, abdominal pain, or vomiting is present.

Dextroamphetamine

Overview

Dextroamphetamine is a CNS stimulant with a characteristic to treat oversedation in patients. This drug is metabolized in the liver.

Description

Dextroamphetamine is a CNS stimulant that is often used for the treatment of ADHD and narcolepsy, and can be used as an adjuvant for obesity treatment.

Dosage and Administration

5-10 mg po bid (8a and 2p)

Drug-Drug Interactions

Dextroamphetamine is contraindicated in patients with a hypersensitivity to other sympathomimetic drugs. Patients who have recently stopped taking nicotine may have a hypertensive sensitivity to dextroamphetamine. Many side effects exist in relation to dextroamphetamine, and the clinician should carefully analyze the patient and the patient's medical history before administering this drug. Acidifying agents, urinary acidifying agents, adrenergic blocks, alkalizing agents, MAO, antihistimines, antihypertensives, corticosteroids, amphetemines, cholorprozine, ethosuximide, haloperidol, lithium carbonate, meperedine, methenamise therapy, norepiunephrine, phenobarbitol, phenytoin, propoxyphene, and others drugs may have drug-drug interactions with dextroamphetamine.

Possible Side Effects

The standard CNS stimulant side effects are common, including insomnia, agitation, restlessness, as well as a variety of adverse effects that are not as common (overstimulation, headache, tremors, and the like). Anorexia and weight loss may occur, GI tract side effects are possible, and vision problems may manifest through the administration of dextroamphetamine.

Precautions

Dextroamphetamine is contraindicated in patients with cardiovascular disease, hypertension, advanced arteriosclerosis, glaucoma, or a hypersensitivity to other sympathomimetic drugs. It should not be used within 14 days of the administration of an MOAI, as elevated, hypertensive blood levels may occur. These stimulants may cause motor disorder in children with ADHD and Tourette's syndrome. Therapy should not be discontinued abruptly due to the possibilities of dependence and withdrawal. Patients who have recently stopped taking nicotine may have a hypertensive sensitivity to dextroamphetamine. Many side effects exist in relation to dextroamphetamine, and the clinician should carefully analyze the patient and the patient's medical history before administering this drug.

Additionally, some formulations of dextroamphetamine contain yellow dye No. 5 (tartrazine), which may cause allergic-type reactions (including bronchial asthma) in certain susceptible individuals. This sensitivity is often seen in patients who have a hypersensitivity to aspirin.

Oversedation: Methylphenidate

Overview

Methylphenidate is a CNS stimulant with a characteristic to treat oversedation in patients. This drug is metabolized in the liver. It has a high potential for abuse.

Description

Methylphenidate is a CNS stimulant that is often used for the treatment of ADHD.

Dosage and Administration

2.5-10 mg po (bid at 8a and 2p) q4 h during day

Drug-Drug

Methylphenidate is contraindicated in patients with a hypersensitivity to other sympathomimetic drugs. It should not be used within 14 days of the administration of an MOAI, as elevated, hypertensive blood levels may occur. Patients who have recently stopped taking nicotine may have a hypertensive sensitivity to methylphenidate. Many side effects exist in relation to methylphenidate, and the clinician should carefully analyze the patient and the patient's medical history before administering this drug. Guanithidine, pressor agents, anticoagulants, anticonvulsants, phenobarbitol, phenytoin, phenylbutazone, and TCA's may have drug-drug interactions with methylphenidate.

Possible Side Effects

The standard CNS stimulant side effects are common, including insomnia, agitation, restlessness, as well as a variety of adverse effects that are not as common (overstimulation, headache, tremors, and the like). Anorexia and weight loss may occur, GI tract side effects are possible, and vision problems may manifest through the administration of methylphenidate.

Precautions

This drug is contraindicated in patients suffering from anxiety, tension, and nervousness due to the drug's tendency to aggravate these symptoms. Methylphenidate is contraindicated in patients with cardiovascular disease, hypertension, advanced arteriosclerosis, glaucoma, or a hypersensitivity to other sympathomimetic drugs. It should not be used within 14 days of the administration of an MOAI, as elevated, hypertensive blood levels may occur. These stimulants may cause motor disorder in children with ADHD and Tourette's syndrome. Therapy should not be discontinued abruptly due to the possibilities of dependence and withdrawal. Patients who have recently stopped taking nicotine may have a hypertensive sensitivity to methylphenidate. Many side effects exist in relation to methylphenidate, and the clinician should carefully analyze the patient and the patient's medical history before administering this drug.

Dry Mouth

Guiding Principles

Dry mouth is a common side effect of opioids and tricyclics and may impact patient adherence to therapy. Treatment of dry mouth includes use of nonpharmacologic and pharmacologic therapies and modalities. Some OTC medications, caffeine, tobacco, and commercial mouthwash increase dryness and should be avoided. Spicy, salty and acidic foods should also be avoided as they may increase irritation to the mouth.

Persistent dry mouth can result in loss of dental structure or endodontic problems. Be aware that patients may choose to discontinue medications because of dry mouth.

Treatment choices include decreasing the use of any non-analgesic causal agents, such as sugar or caffeine. Initially initiating a trial of nonpharmacologic interventions, such as increasing fluid intake (unless contraindicated), offering mouth lubricants, or suggesting sugarless gum or candy can be helpful. Prescribing Pilocarpine (Salagen) 5-10 mg PO tid is also an option, but should be indicated only after nonpharmacologic attempts have had little result.

TREATMENT CHOICES

Decrease use of any non-analgesic causal agents
(i.e.: sugar, caffeine)
Initiate trial of nonpharmacologic interventions:
a) Encourage increase in fluid intake unless contraindicated
b) offer commercial and ‘home-made’ mouth lubricants
c) suggest sugarless gum or candy
Pilocarpine (Salagen) 5-10 mg po tid

Reassessment

Be aware that patients may choose to discontinue their medication treatment because of dry mouth. By instigating non-pharmalogical treatment first, rather than immediately considering pilocarpine, the clinician can offer solutions that the patient can perform themselves, such as chewing gum or using mouth lubricants. Dental hygiene is extremely important for patients with dry mouth to ensure that no endodontic complications set in during treatment.

Dry Mouth: Pilocarpine

Overview

Pilocarpine is a direct-acting cholinergic agonist that is indicated for glaucoma, intraocular tension, and other ocular uses. It is also useful for dry mouth. It is metabolized by the liver.

Description

Pilocarpine stimulates muscarinic cholinergic receptors in the patient, usually to specifically benefit ophthalmic centers. Pilocarpine can negate the side effect of dry mouth in most patients, but with the danger of adverse side effects.

Drug-Drug Interactions

Beta-adrenergic antagonists, anticholinergic agents.

Dosage and Administration

5-10 mg po tid

Ongoing Monitoring

For as long as dry mouth persists. keep a daily check on the quality of the patient's teeth and gums.

Possible Side Effects

Ocular stinging, burning, tearing. and ciliary spasms. System-wide effects include nausea, vomiting, hypertension, salivation, sweating, tachycardia, bronchiolar spasm, and pulmonary edema. A significant amount of contraindications and life-threatening acute side effects are possible with the use of pilocarpine.

Precautions

Contraindications include acute iritis, uncontrolled asthma, and a hypersensitivity to pilocarpine. Reasonable precautions need to be taken for a history of retinal detachment, poor nighttime driving, chronic obstructive pulmonary disease, chronic bronchitis, or anxiety disorders. When systematic cholinergic toxicity occurs, it must be recognized and treated quickly and effectively. Failure to do so can result in patient medullary paralysis, excessive salivary and tracheo-bronchia excretions, bronchospasm and laryngospasm, all of which can cause respiratory failure. All symptoms can be reversed by atropine, an anticholinergic drug. Pilocarpine has a wide variety of uses, but can lead to life-threatening situations if care is not maintained to research, administer to, and monitor the patient responsibly.

Delirium

Guiding Principles

Delirium can be due to either the direct effects of cancer on the CNS, or to indirect CNS effects of the patient's disease or treatments. The clinician should check the patient's medications, electrolyte imbalance, or check for the failure of a vital organ or system, possible infection, preexisting cognitive impairment or symptoms of dementia.

Patients with an acute onset of agitation, impaired cognitive function, altered attention span, or a fluctuating level of consciousness should be evaluated for delirium. Patients experiencing delirium requires a caregiver to be in attendance for assistance with medication management and safety. While delirium is part of the dying process, the clinician must differentiate whether the symptoms result from a side effect or if they are a result of the patient's terminal status.

To gain an accurate assessment for the delirium side effect, the clinician should perform a work-up for brain metastasis and for sepsis. Concurrent drug interactions should be researched, and electrolyte tests/liver and kidney function tests should be performed. Psychiatric evaluation should be considered.

Drugs used to treat Delirium
DRUG CHOICES

| | |
|---|---|
| Haloperidol | 0.5-1.0 mg po, IM, or IV |
| | Can be repeated every 30 to 45 minutes until |
| | symptom control is achieved then q 4-6 hrs prn |
| | Although not approved for IV use haloperidol is |
| | commonly and safely administered by the IV route. |
| Thioridazine | HCL 10-25 mg po tid |
| Chlorpromazine | 25-50 mg po q 6 hrs |
| Pentobarbital | 20-40 mg (use elixir to achieve this dose) |
| | po or 30 mg PR bid-qid |

Patient contact for the actively delirious patient should be daily to several times a day. If delirium decreases with treatment contact qd—3× week until stable. If the delirium is suspected of being opioid-induced, decrease the dosage of the opioid by 30%-50% if the pain level will allow. Maximize co-analgesics for opioid sparing effect. Reassess the situation q 24-48 hours. If the delirium persists conduct sequential opioid trials, review the patient's drug history, and a parenteral route may be required acutely. When changing from one opioid to another, be sure to utilize equianalgesic conversion, and reassess every 24-48 hours.

If the clinician suspects that opioid delirium still persists after the first measures have been taken, and/or aggressive therapy is desired, consider Inpatient or Ambulatory Care Evaluation. Evaluate for regional anesthetic techniques for opioid sparing effect.

Delirium is a part of the dying process and must be differentiated from an acute problem before treatment is initiated. If the delirium persists and terminal sedation is desired, titrate antipsychotics to effect. Initiate and titrate benzodiazepines and barbituates to effect to treat the delirium.

Reassessment

Patient contact for the actively delirious patient should be daily to several times a day. If the delirium state decreases with treatment, continue to keep in contact with the patient at least three times a week until they are stable.

Haloperidol

Overview

Haloperidol is a butyrophenone with the unique quality of being an antipsychotic. Haloperidol has strong anticholinergic effects, and is often used to treat schizophrenia and other similar disorders. Haloperidol can be used to treat delirium.

Description

Haloperidol is usually administered for the management of psychotic behavior, episodes, or disorders.

Drug-Drug Interactions

Anticonvulsants and phenindione (?) may result in drug-drug interactions.

Dosage and Administration

0.5-10 mg, po, IM, or IV* can be repeated every 30 to 45 minutes until symptom control is achieved then q 4-6 hrs prn. (Although not approved for IV use haloperidol is commonly and safely administered by this route.)

Ongoing Monitoring

If the clinician suspects that the patient is experiencing delirium caused by opioid treatment, the clinician should decrease the dose of the opioid by 30%-50% if the patient's pain level allows. Maximize co-analgesics for opioid sparing effect, and reassess the situation every twenty four to forty eight hours.

Possible Side Effects

Tardive dyskinesia, extrapyramidal systems, tardive distonia, and a variety of CNS effects are possible. Careful monitoring is required of the patient, as there have been cases of sudden death in patients being administered haloperidol. While the exact reason or relationship between haloperidol and the patient death has not been established, it is a possibility that should be carefully watched for.

Precautions

Haloperidol is contraindicated for patients that have or are likely to develop extrapyramidal symptoms, and is also contraindicated for patients with CNS depression or are in a comatose state. Asians have a stronger sensitivity to Haloperidol, and dosage should be adjusted accordingly.

Myoclonus

Guiding Principles

Myoclonic activity is known to be associated with opioid drugs and/or their metabolites. Myoclonic spasms are generally not a precursor to a seizure, though mild myoclonic jerks are usually more troubling to the family or caregiver than they are to the patient. Treatment of myoclonus is initiated based on patient safety concerns and patient determination of whether the symptom is bothersome. However, severe myoclonus (ie: >6 jerks/minute) should be evaluated for effect on patient safety.

For assessment of myoclonus, high dosage opioid treatments are likely the cause. Performing liver and kidney tests, and checking electrolytes are standard work-up procedures. Checking for concurrent drug interactions and performing a neurological exam is also recommended. Myoclonus should decrease with treatment over the course of two to three weeks. For more moderate to severe cases, contact with the patient should be made daily to several times a day.

Drugs to treat Myoclonus

| Drug | Dose and schedule | |
|---|---|---|
| Clonazepam | 0.5-4 | mg PO bid |
| Diazepam | 2-10 | mg PO/IV qid |
| Lorazepam | 0.5-4 | mg PO/IV tid |
| Baclofen | 10-20 | mg PO tid |

If myoclonus persists, review the patient's drug history. Maximize co-analgesics for opioid sparing effect and consider conducting sequential opioid trials. While the oral route is preferred, parenteral may be required for acute pain. Once the pain control has become adequate, reduce the dose of the opioid by 50%. Reassess every twenty four to forty eight hours.

If myoclonus still persists, consider Inpatient or Ambulatory Care Evaluation. Emergent therapy may require intravenous midazolam 0.5-1.0 mg IV q 10 minutes—titrate to effect, monitor oxygen saturation. Midazolam drip may be necessary during acute opioid toxicity and during opioid conversion. Evaluate for regional anesthetic techniques for opioid sparing effect. Consider use of local anesthetic only or local anesthetic/fentanyl for pain control during acute opioid withdrawal.

Reassessment

Contact for the patient with moderate to severe myoclonus should be daily to several times a day. If myoclonus decreases with treatment contact qd to 2-3× week until resolved.

Clonazepam

Overview

Clonazepam is a benzodiazepine derivative used to treat a variety of seizures. Peak plasma levels will take place within one day of administration. Clonazepam has a rapid onset.

Description

Clonazepam suppresses the spike and wave discharge in absence seizures and decreases the amplitude, duration, frequency, and spread of discharge in minor motor seizures.⁸ ⁸Nursing Management in Drug Therapy. Lippincott, Williams & Wilkins 1999

Dosage and Administration

4 mg po bid

Drug-Drug

Do not mix with alcohol due to the danger of CNS depression. Clonazepam has a tendency to increase in strength when in drug-drug reactions with cimetidine, omeprazole, disulfiram, and oral contraceptives. Phenytoin, carbamazepine, phenobarbitol, MAO, CNS depressants, TCA's, and anticonvulsants may have drug-drug interactions with clonazepam.

Ongoing Monitoring

Possible Side Effects

A variety of CNS depression effects are likely, including sedation, drowsiness, lightheadedness, and other symptoms. This drug has many side and adverse effects, so research should be conducted carefully to ensure the safety of the patient while Clonazepam is being administered.

Precautions

Clonazepam may cause seizures in patients prone to seizure disorders. Clonazepam is contraindicated if the patient has liver disease, renal failure, acute narrow-angle glaucoma, and a sensitivity to benzodiazepines or psychoses. Elderly and debilitated patients may have a prolonged effect from Clonazepam due to the relative inefficiency of their liver to process Clonazepam—beware of overdosing

Lorazepam

Overview

Lorazepam is an anxiolytic that causes sedation, induction and maintenance of anesthesia and anterograde amnesia of perioperative events.⁹ Lorazepam is absorbed from the GI system. ⁹Nursing Management in Drug Therapy, Lippincott. Williams & Wilkins 1999

Description

Lorazepam is used to treat a variety of side effects, including nausea and vomiting caused by drugs used in interaction with chemotherapy. It is metabolized in the liver. Lorazepam also provides anti-convulsant effects in the patient.

Drug-Drug Interactions

Drug-drug interactions between Lorazepam and benzodiazepines may cause psychotic episodes, and a variety of drug-drug interactions exist that will increase or decrease the effects of lorazepam in relation to other drugs. Flumazenil and lorazepam will result in the lorazepam's effects being reversed.

Dosage and Administration

1-4 mg po tid. Antacids will decrease the efficacy and absorption rate of Lorazepam.

Ongoing Monitoring

Possible Side Effects

The drug causes CNS depression, and should not be mixed with other drugs that are capable of CNS depression for fear of adverse side effects like respiratory depression. Less common effects include sedation, GI problems, changes in mood, changes in mental capability, hallucinations and impaired motor control.

Precautions

Lorazepam is contraindicated in patients with psychoses, a sensitivity to the drug category, or acute narrow-angle glaucoma. A variety of drug-drug interactions exist that will increase or decrease the effects of lorazepam in relation to other drugs. The sedative effects of lorazepam can be reversed by flumazenil. Patients with sleep apnea or other breathing obstruction causes may have difficulty with respiration while taking Lorazepam. Toxicity is possible in patients with liver problems, and more elderly patients should be initiated on smaller doses.

GI Distress

Guiding Principles

The use of NSAIDS has been associated with both minor and major GI toxicities. Be especially aware of potential GI bleed. Serious side effects are not always preceded by minor side effects. Patients should be taught to report any GI disturbances, no matter how minor.

When trying to account for the GI Distress side effect, perform a diagnostic work-up as appropriate and/or perform a GI consultation. Rule out other causes, assess oral intake and timing of NSAID indigestion, and assess concurrent drug interactions. Reassess three to seven days after the initiation of patient's therapy.

Drugs to treat GI Distress

| Drug Choice | Dosage and Schedule | |
|---|---|---|
| Ranitidine | 150 | mg PO bid |
| Cimetidine | 300 | mg PO qid |
| Omeprazole | 20 | mg PO qd |
| Misoprostol | 200 | mcg PO qid |
| Famotidine | 20 | mg PO/iv qd - bid |
| Nizatidine | 150 | mg PO bid |
| Lansoprazole | 30 | mg PO qd |

Diclofenac 50/75 mg with Misoprostol 200 mcg now available as combination product, though it is contraindicated in pregnant women.

If the patient is suffering GI Distress, and it is suspected that the problem is NSAID-induced, consider a drug change to one that will have the same analgesic effect, but will cause less GI-Distress. Several NSAIDS including salsalate, choline magnesium trisalicylate, and nabumetone have reportedly less GI toxicity. A trial of Diclofenac/Misoprostol combination product could be attempted if side effects persist, or contraindicating NSAIDS for the patient altogether. Watch for drug interactions with cimetidine and consider dosage reduction of the H2 blockers in patients with renal dysfunction.

Reassessment

Re-assess the patient's condition within 3-7 days of initiation of therapy. Changing to drugs such as salsalate, choline magnesium trisalicylate, and nabumetone have reportedly less GI toxicity. A trial of diclofenac/misoprostol could be effective. If GI Distress persists throughout all of these changes, discontinue NSAIDs.

Misoprostol

Overview

Misoprostol is a synthetic used to prevent NSAID-induced ulcers in patients with a high risk of ulcers by reducing acid secretion during digestion. Misoprostol is often used in combination with other drugs to reduce the chance for GI distress and toxicity.

Initiation

Taking this drug with any food delays its absorption rate. Antacids should not be simultaneously administered with misoprostol, as misoprostol will reduce the antacid's efficacy.

Description

Diclofenac is an acetic acid that has a long half-life, and is very effective for reducing the chance of toxicity due to the reduced need for constant dosage. This drug is effective for rheumatoid arthritis and osteoarthritis. A drawback of the drug is its high cost. Misoprostol protects the stomach lining, decreasing the chance of ulcers in patients that are taking NSAIDS. Misoprostol is most effective if taken three to four times daily.

Dosage and Administration

200 mcg po qid

Drug-Drug Interactions

Phenobarbitol, lithium, antacids, diuretics, aspirin, digoxin, antihypertensive agents, warfarin, oral hypoglycemics, methotrexate, and cyclosporin may have drug-drug interactions with Misoprostol.

Possible Side Effects

Misoprostol will frequently cause diarrhea, abdominal pain, nausea, cramps, vomiting, dyspepsia, flatulence, constipation, and headaches. Gynecologic problems include spotting, cramps, and menstrual disorders.¹⁰ ¹⁰Nursing Management in Drug Therapy. Lippincott, Williams & Wilkins 1999

Precautions

As the acetic acids are highly protein-bound, they may displace other protein-bound drugs, which can result in toxicity and reduction of analgesia. Misoprostol should not be given is contraindicated in pregnant women, as the drug will cause miscarriage and uterine contractions.

Nizatidine

Overview

Nizatidine is an anti-secretory agent that increases the effectiveness of salicylates in patients and reduces the amount of gastric acid. It is often used to treat ulcers. Nizatidine is partly metabolized in the liver.

Description

Nizatidine is a drug comparable to the cimetidine, a drug that is used to protect against peptic ulcers. Nizatidine is not used for treating pathological hypersecretory conditions and preventing upper GI bleeding, aspiration pneumonitis, and stress ulcers.¹¹ ¹¹Nursing Management in Drug Therapy. Lippincott. Williams & Wilkins 1999

Dosage and Administration

150 mg po bid

Drug-Drug Interactions

High doses of aspirin may have drug-drug interactions with Nizatidine.

Ongoing Monitoring

Possible Side Effects

While nizatidine causes fewer adverse effects than cimetidine, but is more likely to cause constipation. With the administration of nizatidine comes the effects of sweating, pruritus, and urticaria.

Precautions

In drug-drug interactions antacids, metoclopramide, and anticholinergics affect nizatidine's efficacy. Because nizatidine does not inhibit hepatic metabolizing enzymes, there are less drug interactions. Nizatidine should be administered in reduced dosage to patients with renal insufficiency.

Omeprazole

Overview

Omeprazole is an anti-secretory drug (a substituted benzimidazole) that suppresses the enzymes that produce gastric acid. This drug is used in the treatment of ulcers and patient GI distress.

Description

Omeprazole has a half-life of about an hour. Patients with hepatic problems may have stronger effects from Omeprazole due to an increased first-pass effect. It does not have anticholinergic properties.

Dosage and Administration

20 mg po qd to bid

Drug-Drug Interaction

Diazepam, phenytoin, warfarin, cyclosporine, disulfiram, benzodiazepines, ketoconazole, ampicillin esters, iron salts, cisapride, pimozide, and terfenadine may cause drug-drug interactions.

Possible Side Effects

Most patients tolerate Omeprazole. Side effects of headache, diarrhea, constipation, abdominal pain, nausea, vomiting, dizziness, cough, rash, asthenia, upper respiratory infection, and back pain.

Precautions

Omeprazole should be administered carefully to the elderly. Additionally, Omeprazole can cause diazepam, warfarin, and phenytoin to have a prolonged effect.

Ranitidine

Overview

Ranitidine is an anti-secretory agent that reduces the amount of gastric acid. It is often used to treat ulcers. Ranitidine is partly metabolized in the liver.

Description

Ranitidine is a drug comparable to the cimetidine, a drug that is used to protect against peptic ulcers. Ranitidine can be used for treating pathological hypersecretory conditions and preventing upper GI bleeding, aspiration pneumonitis, and stress ulcers.¹² Ranitidine is especially good for the prevention of GI damage from the use of NSAIDS, and it prevents erosive esophagitis from occurring. ¹²Nursing Management in Drug Therapy. Lippincott, Williams & Wilkins 1999

Dosage and Administration

150 mg po bid

Drug-Drug Interactions

Warfarin and triazolam are both drugs that may potentially cause drug-drug interactions with Ranitidine.

Possible Side Effects

Some tests have shown that Ranitidine causes fewer side effects in patients than cimetidine.

Precautions

In drug-drug interactions antacids, metoclopramide, and anticholinergics affect the efficacy of ranitidine. It increases the effect of warfarin and sulfonylureas and decreases the effect of diazepam. Ranitidine should be administered in reduced dosage to patients with renal insufficiency.

Alternative Routes

Parenteral Therapy

Guiding Principles

A patient is a candidate for parenteral therapy when less invasive routes are not effective in controlling the patient's pain. In the event that the patient is suffering severe intermittent pain requiring fast-acting analgesia, or is in need of rapid titration due to extraordinary pain levels, parenteral therapy can be the key. While this route is usually performed in the hospital environment, if there is a responsible caregiver available parenteral therapy can be administered in the home.

There are three kinds of pareinteral treatment choices available:

Intravenous (IV): Continuous intravenous infusions of opioids produce stable blood levels of drug, reducing the frequency and intensity of “peak” and “trough” levels associated with recurrent pain. Risks include potential for infection of IV site. Options also include implantable central venous access catheters, ports, and pumps. These devices are more expensive and require surgical implantation.

Subcutaneous: Intermittent and continuous subcutaneous infusion is an effective route for opioid administration. It provides analgesia that is equivalent to that achieved by the intravenous route. Site options most commonly used are the subclavicular region, anterior chest or abdomen. Needle placement is easily achieved and managed. Infusion generally limited to 2 cc/hr maximum infusion rate. Concern over irregular absorption due to uneven tissue perfusion exists with the subcutaneous route.

Intramuscular (IM): This method is undesirable for long term use because it is painful for the patient and difficult to manage at home. Absorption of the medication via this route is inconsistent. IM medication needs to be repeated regularly because of the short duration of action of most opioids. Intervals of at least 4 hours are common.

Reassessment

See Pain Crisis Intervention for acute dosing strategies.

The clinician should titrate the dosage and schedule according to the level of pain intensity. On the 1-10 pain scale, if the pain is less than 4, escalate up by 10% of hourly dose no more than once in a 24 hour period. If the pain level is from 4 to 6, escalate up by 30% of hourly dose two to three times per day. If the pain level is from 7 to 10, escalate up by 50% of hourly dose every three to four hours. Monitor the patient carefully during any accelerated titration process, and make sure that the patient knows to monitor and report all signs and symptoms of complications and/or side effects.

Regional Blockade

Guiding Principles

Regional blockades are useful for the immediate relief of regional pain, and can greatly reduce the need for opioids, allowing for a reduction in side effects. The key goal of regional blockade therapy is to interrupt nociceptive pathways without producing significant impairment of motor function. While the process of blocking neurotransmission to provide analgesic effect can be very effective for treating pain, the process may lead to temporary side effects that occasionally become long-term—such as motor weakness, hypotension, and bladder control problems.

Refer the patient for consultation to an anesthesiologist with training in regional anesthesia techniques, and be aware that once the block has taken effect there may be an increase in the side effects of the opioids still being used. Slowly titrate the opioids down to reduce the actualized or potential side effects.

Assessment

A patient is a candidate for regional blockade when they are experiencing regional pain, experiencing intractable pain with or without side effects, or experiencing neuropathic pain limited to two dermatomes and accessible to blockade. Neurolytic procedures are used only in patients with a limited prognosis (i.e.: to avoid long term complications of neuronal regeneration) and performed in regions without critical motor function. Be sure to consult an anesthesiologist who has training with regional blockades for all details regarding this procedure.

Treatment choices and uses:

Epidural injections of local anesthetic and steroid admixtures are used to treat pain caused by nerve root compression and irritation.

Diagnostic blocks with local anesthetic are used to determine neurolytic block probability of success.

Peripheral nerve destruction can be accomplished by the injection of ethanol, phenol, or other neurolytic agents at sites where previous test injection of local anesthetic have produced pain relief. Neurolysis provides pain relief for three to six months; pain may return in the same area due to tumor growth beyond nerve distribution.

Sympathetic ganglion destruction (i.e. celiac plexus) can be accomplished with alcohol or phenol. There is a small risk of paralysis.

Reassessment

If the patient reports a decrease in pain after the procedure, opioids should not be stopped abruptly due to risk of withdrawal. Additionally, patients receiving anti-tumor therapy or regional techniques may experience decreasing pain and a sudden increase in side effects related to pain medication. Titrate downward by 25-50% of the daily dose every one to two days until discontinuation or return of pain.

Rectal Opioids Administration

Guiding Principles

The clinician should use the rectal route when the po route is either contraindicated or a change in level of consciousness makes administering po medications difficult. The rectal route is significantly less expensive than the parenteral alternatives, and can be placed in a colostomy or a similar stoma.

Route limitations include decreased acceptance by patient and family, a relatively slow onset of action, and variable absorption. A compounding pharmacist may combine opioids with a variety of medications in a single suppository. Check with your local pharmacy for potential formulations and availability.

Assessment

The patient is a candidate for rectal administration if the stomal or rectal effluent is slow enough to allow for absorption, if there is a caregiver to assist with drug administration, and if the patient and family accept this prescribed route. Rectal administration is contraindicated in any patient if there is the presence of lesions of the anus, obstruction, copious discharge, and severe or ongoing constipation.

Rectal Route Drug Choices

Conversion from the oral route to the rectal route starts with same dose and frequency and is titrated to individual patient response. Vehicles can be either liquid or solid, and may include aqueous solutions, suppositories, and commercially available tablets intended for oral use (including time-release preparations). Contact your pharamacist for assistance in compounding suppositories.

Reassessment

Aggressive titration of drugs may be difficult due to the limited availability of commercial preparations in varying strengths. Additionally, limited space in the rectum generally precludes insertion of more than trio suppositories. Be sure to have the patient notify the clinician of any changes in bowel habits or loss of analgesic effect.

Spinal Administration

Guuiding Principles

Spinal Administration is useful for the treatment of persistent severe pain, particularly in the presence of side effects. A temporary percutaneous epidural or intrathecal catheter may be placed for a trial; consult your anesthesiologist or neurologist for information on this procedure.

All medication for epidural and intrathecal and equipment used for epidural administration should be free of toxic preservatives and approved for epidural use. Consult your pharmacist before injecting or infusing anything into an epidural catheter.

For pain of neuropathic origin, addition of bupivacaine or clonidine provides better pain relief than opioid alone. The skillful manipulation of epidural bupivacaine can create pain and sensory blockade while sparing motor function. The combination of opioid and bupivacaine is particularly helpful in the patient who continues to have severe pain with side effects of escalating opioid.

Admixtures may provide synergism that allows decreased dosages and toxicity for all medication in the admixture. Clonidine may be added to opioid and/or low concentration bupivacaine to achieve control of nenropathic pain with less risk of motor loss. Neurological procedures may be considered.

PCA administration allows rapid response to patient incident related and breakthrough pain. Bolus dosing should not exceed 20% of the hourly dose.

Assessment

The patient is a candidate for epidural/intrathecal therapy if there is the presence of severe pain with or without unmanageable side effects that would be affected by a regional blockade, and whether there has been a previous adequate trial of a less invasive opioid or co-analgesic administration (success in the oral, rectal, transdermal, or parenteral routes). A reliable caregiver is required, and

Drug Choices
Opioid

Effective for treatment of nociceptive pain, less effective for of severe
neuropathic pain. Convert current systemic opioid to epidural opioid (MS
10 mg IV = 3 mg epidural). Convert epidural to intrathecal opioid (MS
3 mg epidural = MS 0.3 mg intrathecally).
Some epidural opioids such as morphine may be administered as individ-
ual bolus dose injections on a scheduled basis (i.e. q8h, q6h). This
method is less costly.
Titrate epidural opioid based on pain intensity report:

| | |
|---|---|
| Pain <4 | escalate up by 10% hourly dose daily |
| Pain 4-6 | escalate up by 30% of hourly dose 2-3x per day |
| Pain 7-10 | escalate up by 50% of hourly dose every 3-4 hrs |

Titrate intrethecal opioid 10-30% daily for pain >3
Local anesthetic (epidural)
Effective for treatment of pain with nociceptive and neuropathic origin.
A starting concentration of bupivacaine for chronic administration is
0.11%.
Titrate epidural bupivacaine solution to pain relief using the following
general guidelines:

| | |
|---|---|
| Pain >4 | elevate bupivacaine concentration by 0.05% |
| Pain 4-6 | elevate bupivaccaine concentration by 0.01% |
| Pain 7-10 | elevate bupivacaine concentration by 0.03% |

Clonidine
Particularly effective for pain of neuropathic origin.
Tri-mixtures of opioid and bupivacaine with clonidine are possible. Effi-
cacy of agents (as well as side effects) may be synergistic.
Epidural clonidine cannot be administered by bolus injection
due to cardiovascular side effects.
Intrathecal clonidine os not FDA approved but is reported to be effective
in the literature.
In patients with significant dehydration, baseline hypotension, or long
standing hypertension consider IV fluids prior to starting epidural
clonidine.
Epidural conidine is initiated at 10-30 mcg per hour continuous infu-
sion. Patients need to be monitored for cardiovascular changes such as
postural hypotension and bradycardia for the first 24 hours and following
each dosage change.
Titrate clonidine no more than 10-15% of dose per 24 hour period to
avoid hypotension/bradycardia. Epidural clonidine commonly is dosed at
20-50 mcg/hr and has been utilized up to 100 mcg/hr when slowly.

Reassessment

Monitor the patient for sedation (opioid/clonidine), myoclonus (high dose spinal opioids), symptomatic orthostasis (bupivacaine/clonidine), and/or dermatomal numbness/weakness (bupivacaine).

Volumes of drug administered into the epidural space should not exceed 30 ml per hour. Optimal volume of infusate should be determined by epidurogram. Lower infusion rates (<10 ml/day) and fewer intrusions into the infusion system are associated with fewer infections. Be sure to instruct the patient to notify clinician of any sudden increases in pain, pain on injection, pain in the spinal region, or local signs of infection. Be aware that fever is not always present during episodes of epidural infection.

Rebound hypertension can result from abrupt withdrawal of epidural clonidine therapy. All patients require gradual weaning if therapy is to be discontinued. It is recommended that systemic clonidine be added during the taper down process especially if the patient has pre-existing hypertension (see process below).

A suitable gradual reduction schedule for epidural clonidine is a 20% reduction every one to two days; monitor the patient's blood pressure before each rate change. In the event that abrupt withdrawal is required, cover the patient concurrently with oral and transdermal medication. Taper the oral dose by 20% daily, and once again monitor the blood pressure daily during titration. Neurological procedures, including cordatomy and others, are non-reversible and usually less acceptable to patients. These may be reconsidered if the spinal protocol fails.

| Epidural dose | Transdermal dose | Oral dose |
|---|---|---|
| >700 mcg/day | TTS-3 | 0.1-0.2 mg po q6-8 hr |
| up to 700 mcg/day | TTS-2 | 0.1-0.2 mg po q6-8 hr |
| <200 mcg/day | TTS-1 | 0.1-0.2 mg po q6-8 hr |

Oral Transmucosal Fentanyl Citrate (OTFC) Administration

Guiding Principles

The medication is administered on a stick swabbed over the gums and other oral mucosal membranes; medication that is inadvertantly swallowed is broken down by the liver, and does not provide much analgesic effect. This route provides short-acting relief for breakthrough pain; fentanyl citrate is currently the only medication available for administration via this route. Caution must be taken when using this medication in the presence of children as units may be mistaken for a food substance.

Assessment

A patient is a candidate for OTFC if they are opioid tolerant (currently taking as much as 60 mg morphine daily, 50 mcg of transdermal fentanyl hourly, or equainalgesic opioid equivalents for one week or longer. OTFC is excellent for patients that are experiencing breakthrough or incident pain that requires rapid short-acting analgesia, are not experiencing moderate to severe oral mucositis. The patient must be educated on the precautions that must be taken when using this medicine in the presence of children. Instruct patient to dispose of partially used or unused units by dissolving medication under running hot water or by placing in child-resistant container.

Drug Choices

| | |
|---|---|
| Available unit strengths: | 200, 400, 600, 800, 1200, 1600 mcg/unit |

After the patient receives instruction on how to use the OTFC units, therapy may be initiated with 200 mcg units. The dose may be repeated 15 minutes after the previous unit is consumed, but with a limit of no more than two units per pain episode. If more than one unit is required to attain relief, increase the dose to the next highest strength; a successful dose is determined when adequate relief is achieved with a single OTFC unit.

Reassessment

Consumption of more than four units per day indicates a need for titration of ATC opioid or an increase of OTFC to next dosing strength.

Transdermal Opioids Administration

Guiding Principles

There are many factors that can influence absorption of medication by this route. These include the presence of fever, the amount of the patient's body hair, the vascularity of the underlying tissue, circulatory compromise, and the difficulty from the patient having low subcutaneous fat stores. Individual patient reaction to the patch adhesive, medication dosing, and efficacy will vary due to these factors.

Hair located under the patch should be clipped and not shaved due to potential increases in skin irritability. By no means should the patch be cut or torn; do not cut the patch in order to adjust the dose, as this will destroy the delivery system and cause the patient to receive extremely high doses of fentanyl. The patient may shower or swim with the patch on, but they should not stay for extended periods of time in baths, hot tubs, or saunas where the skin temperature could be dramatically raised and effect the patch's delivery system. All patients should have a short-acting opioid available to manage breakthrough pain, as the patch is only suitable for the treatment of continuous pain. Obviously the patch is not appropriate for patient's needing rapid titration. For the opioid naïve patient initiate with a patch that has a dose strength of 25 mcg/hour; monitor efficacy and side effects as drug levels rise initially over the next 12-18 hours.

Assessment

The patient is a candidate for transdermal fentanyl if there is the presence of continuous pain, if they could gain benefit from the pharmacologic profile of fentanyl (less constipation, less frequent dosing schedule), and if there is the absence of rapidly escalating pain. If the patient has an inability to tolerate oral or rectal routes, the transdermal route may be an excellent alternative. The patient must have a negative history of sensitivity to skin adhesives.

Transdermal fentanyl is not the best choice when the patient is suffering from uncontrolled pain requiring rapid titration, when they have fever or diaphoresis, or extreme cachesia.

DRUG CHOICES

| | |
|---|---|
| Available patch strengths | 25, 50, 75, 100 mcg/hr |

The recommended process for initiating the transdermal patch starts with the education and instruction of the patient on how to place the patch. Initiate with an equainalgesic dose using the Equainalgesic Conversion Table. Use short-acting opioids to cover breakthrough pain for up to eighteen hours until the patch has become fully effective. Let the patient know that if the patch is damaged or cut in any way, the controlled drug delivery will not be possible; if they were wearing the patch at the time of the cutting or tearing, they should contact their clinician immediately.

Oral or parenteral analgesics must be continued for a period of 18 hours until a steady rate of transdermal fentanyl delivery is achieved. Replace the patch once every seventy two hours, and ensure that rescue opioids are available for the patient. Only increase the dose at the seventy two hour change mark, and ensure that the patch replacement schedule guarantees that all of the patches are changed simultaneously. If the transdermal patches are being changed throughout the seventy two hour period, it is possible that errors or dose problems may result. Some clinicians suggest dose finding with intravenous fentanyl (for inpatients) and then converting to the transdermal patch.

Administration

Do not use soaps or lotions on skin under patch. Wash with water.

Apply to an area of skin on the upper torso that is not irritated, has not been irradiated, and is free of hair. Hair may be clipped but not shaved so as to avoid skin irritation.

Apply with firm pressure with a warm hand for ≈30 seconds to ensure good seal.

Reassessment

Titrate after seventy two hours to a new dosage equivalent to the amount of breakthrough medication used by the patient. Transdermal fentanyl is not suitable for rapid dose titration due to slow onset and long duration of action. If more than 300 mcg per hour is required, the literature recommends changing to another agent/route. Use the patches with caution if WBC's or platelet counts are low, and in patients with skin irritation due to increased risk of infection/bleeding.

Fentanyl Conversion Table

| Fentanyl (mcg/hr) | po morphine equivalents (over 24-hours) |
|---|---|
| 25 | 0-88 |
| 50 | 89-148 |
| 75 | 149-208 |
| 100 | 209-268 |
| 125 | 269-328 |
| 150 | 329-388 |
| 175 | 389-448 |
| 200 | 449-508 |

Patient and Family Issues

Goals of Care

Palliative medicine is centered on the identification and incorporation of the patient's goals of care. For centuries there has been the perspective that the physician knows best and that treatment planning was the isolated domain of the physician. With the advent of more holistic models of care it has become clear that patient participation is a central component to successful health and wellness. In pain management, although we have very successful options for treatment, it is clear that patients often determine their own “algorithm” of treatment.

Pain and Symptom Management as Adjunctive to Active Treatment

For the cancer patient the treatment of the underlying disease is often a life saving therapy and as such takes precedence over virtually every other aspect of treatment. In some cases patients may ignore pain and symptoms or expect to have to “put up with them” as a part of the fight against the cancer. There is an underlying perception that to survive you have to “fight through” the pain. Many times patients will not want to take the time to report pain because they have such a limited number of minutes to spend with their health care provider. Understandably they want that time to be concentrated on what to do next in the anti-tumor battle. Often times as a provider there is no way to cover all the review of systems necessary to impact all symptoms in the time allotted for the patient visit. It is in this setting that tools can be most helpful in streamlining care. Pain assessment and side effect rating scales, patient diaries, and patient education materials for ambulatory care can facilitate communication and enhance the integration of symptom management into the active treatment of cancer. An institutional commitment to providing quality pain and symptom management during all phases of cancer treatment (i.e. surgical, medical, radiation, rehab) is often necessary to make this goal a reality.

Pain and Symptom Management in the Patient with Controlled Disease

The good news is that cancer is being cured; the bad news is that we are often left dealing with long-term pain conditions as a result of cancer treatment. This category of patients is likely to grow. Neuropathic pain syndromes are highly represented in this group and do constitute a more difficult type of pain to treat in general. Early identification of persistent pain and an “all hands on deck” multidisciplinary approach to long term treatment planning is often needed.

Pain and Symptom Management in the Dying Patient

Hospice has led the way over the last 25 years in prioritizing the patient and family's wishes in end of life care. Patients with advanced disease in the algorithm study had an average of 5 different pain locations, almost all with “mixed” pain character, indicating some soft tissues and bone pain as well as neuropathic pain. The majority of these patients will have multiple other symptoms as well. The single most important feature of pain and symptom treatment planning in this population may be to seek out the patient's priority for treatment—Is it pain? Is it drowsiness? Is it sleeplessness? Prioritizing what can be a long list of physical and psychosocial symptoms puts the patient in control and assists the provider in focusing on the top 2 or 3 problems for any one visit or phone call.

Defining the Patient and Family Priorities

It is certainly the dream of many providers to have the luxury of spending 45 minutes or an hour conferencing with patients and families about the plan of care. In the past we have had social work staff or psychosocial nurse specialists who perform this function in “non-revenue producing” positions that have long since been eliminated or “downsized”. So how do we manage this disengagement of need from resources available without neglecting it completely. Oncologists consistently have to educate patients to weigh choices that need to be made about all aspects of care. We need to be able to put systems in place to allow for continuous reinforcement of education and re-assessment of patient priorities. For example with side effect assessment the algorithm assessment includes the following flow of questions: Do you have nausea? How distressing is it? Is treating this a priority? Generally this kind of targeted assessment will elicit a short list of symptoms that are most problematic for the patient, even if there are in fact many symptoms that are present. Interestingly family members may not always agree with the patient's priorities.

The Integrated Plan of Care

The treatment of pain and symptoms is in fact like a negotiated settlement where the array of possible treatment options at the disposal of the provider must be integrated with the priorities expressed by the patient. Too often this step does not occur for many reasons. The best possible solution is to have some form of a pain management treatment plan in writing—either as a part of a flow chart, a part of a patient education check-off sheet, or a part of a doctor-patient communication tool. This integration of treatment should allow for optimized pharmacologic management options as well as the patient's own personal priorities including nonpharmacologic efforts, herbal remedies, etc. A continuous re-visiting of the plan can dramatically open the lines of communication between patient and provider and prevent poor outcomes that result from miscues and missed opportunities.

Fear of Addiction

Fact vs. Fiction

Drug addiction carries a heavy spectre of dread. It is pervasive. It is not going away any time soon. Several large studies have shown that less than one percent of patients who take opioids for pain relief will become psychologically dependent on opioids. In one pivotal study by Friedman of 24,000 patients treated with opioids for pain, only 7 patients became psychologically dependent (0.03%). It happens so rarely in cancer patients that one can safely say that it is more likely that they will be hit by a train than develop true drug addiction. In England where heroin was routinely used for a time in hospice care (before they concluded that morphine was just as good if not better) at least 2 studies in over 500 patients showed no patients becoming psychologically dependent. Education and reinforcement of these facts will be necessary every day in the average oncology practice.

Patient Education

“Some days I think every patient that walks into clinic is afraid of drug addiction,” one nurse told us, “It's like a full time job just to deal with that”. Much of the misperception is based on the confusion surrounding physical dependence versus psychological dependence. Physical dependence is manifested during abrupt withdrawal from the opioids (and many other agents). Most if not all patients that have been on opioids for more than days to weeks will go through an expected set of “flu-like” symptoms as well as some restless agitation if the drug is stopped too abruptly. This is easily avoided by tapering down slowly (the same way we do with corticosteroids). In other words physical dependence occurs in virtually all patient on chronic opioids. Just the fact that patients are taking opioids for pain relief is not addiction, regardless of the dose or length of time on opioids.

True drug addiction is defined as a psychological dependence or “a pattern of compulsive drug use characterized by a continued craving for an opioid and the need to use the opioid for effects other than pain relief”. Most patients can understand this differentiation when asked the simple question, “If you didn't have pain would you want to take this medicine?”. Generally the quick response is “No! It makes me constipated and sometimes queasy.”

Still a third term that is used frequently is tolerance. Tolerance is a decrease in effectiveness over time with a stable dose of opioids. This phenomenon is being studied aggressively. The NMDA receptor in the central circuitry appears to play a role in mediating opioid receptor binding with chronic use. It does not appear to exist uniformly in all patients on chronic opioids, and in cancer patients it is widely understood that most dose escalations are occurring as a result of tumor progression and not tolerance.

The take home message for patient education is the physical dependence and tolerance to opioid therapy is expected and does not pose a significant problem to our ability to use these agents. Psychological dependence is extremely rare and is characterized by a craving for the non-pain relieving effects of the drug.

Provider Communication

All avenues of communication including physician to patient, nurse to patient, physician to nurse, nurse to nurse, etc. have the capacity to retard or promote what we call “opiophobia” (fear of opioid addiction). The classic one is provider to provider communication involving “the drug seeker”. At the change of shift one nurse reports off that Mrs. J in 601 is a drug seeker. This is a terribly destructive term that stigmatizes the patient and destroys the patient-provider partnership. What is almost always true is that Mrs. J is in fact a pain relief seeker, trying to make people aware that the medication is not covering her pain, or the frequency of the schedule is too long to cover the pain. Another example is the patient who asks the physician for a pain medicine by name. Mrs. J. asks “Dr. Smith can I get Dilaudid instead of Morphine?” Some physicians will be so incensed that they will go out of their way to order anything besides the drug requested. Could it be that the patient is simply operating on experience of what works best for them? Would we question a patient that appears to be knowledgeable about which bronchodilators work best or what insulin configuration is most effective? Margo McCaffrey suggests the following three questions be explored in these types of situation:

If this is a label, not a diagnosis, what has Mrs. J done to cause us to believe she is drug seeking or addicted?

Is there any other way to explain Mrs. J's behavior that seems to indicate drug seeking or addiction? In other words, might she behave in this way for some reason other than drug seeking?

Could Mrs. J be seeking pain relief rather than drugs for nonmedical uses?

Finally there were a number of times during the algorithm study where patients were told by their health care providers that they might become addicted to the opioids. This has a profound impact on patients and should be avoided at all costs. While it is true that there is a slim possibility that substance abuse could occur, this kind of statement from a provider will almost surely directly dissuade the 99% of patients who have nothing to worry about from taking their pain medications.

The Ex-Abuser

The patient with a history or current substance abuse problem can be a significant challenge. The patient with a history of psychological dependence, either with illicit drugs or alcohol, may fall into two difficult management arenas: unwilling to take any opioids because of fear of relapse, or very tolerance to opioids requiring huge doses to obtain relief. We interviewed patients who felt that despite an understanding of physical versus psychological dependence, their previous history of substance abuse absolutely precluded their willingness to take opioids. Often in these situations patients and providers must negotiation for non-opioid approaches (co-analgesics, nonpharmacologic options, interventional techniques) or risk a situation where the provider continues to prescribe and the patient simply does not take the medication and suffers in silence.

The patient with “super-tolerance” may be in jeopardy of not getting enough pain relief because the provider is not comfortable prescribing high doses of opioids. It is not uncommon for these patients to get inadequate doses of opioids, but high doses of benzodiazepines or antihistamines to try to “boost” the opioid in lieu of just simply increasing the opioid to adjust for tolerance. A pain team consult in this situation (when available) is helpful. The bottom line is the patient will likely experience more pain relief and significantly less side effects if they are just given higher doses of opioids.

Managing the cancer pain patient with an active illicit drug dependence is perhaps the greatest challenge, both pharmacologically as well as bioethically. Consults to the pain management team, an addictionologist, and even the ethics committee will make treatment planning easier. The biggest dilemma is—if the illicit drug use continues, is the provider willing to prescribe pain medication. If support resources are not available the basic underlying principles for treatment are:

Establish a written contract with the patient that includes a one prescriber, one pharmacy clause

For outpatients supply only limited quantities of opioid for frequent pick-up (from daily to not greater than once a week)

Optimize co-analgesics and include their utilization in the contract

If abstinence is a part of the contract, random urine testing may be necessary to assure compliance

Pain Interference

Pain interference is like a “mini quality of life” check on the patient who is living with pain. Patients tell us that they often make decisions about taking their pain medication based on functionality that can be enhanced by pain relief or diminished by side effects. The pain interference scale is located in the Tools section of this manual. In general the pain interference scale asks patients to quantify how much the pain interferes with walking, working, sleeping, sitting, relationships with other, etc. Although this level of assessment may not be possible with each patient contact, some check-in with the patient and family in this area should occur on a regular basis. Tailoring of analgesic therapy can be done to accommodate priority functional areas such as taking a walk with a spouse or doubling the bedtime dose of opioid if pain is particularly bad at night.

Pain Tolerability

One interesting finding from our algorithm work has been that patients often make decisions about their pain medication based on a level that they perceive as being “acceptable” or “tolerable”, below which they may choose not to medicate. Results from the study showed that the average level of pain “tolerability” for 200 cancer patients was a 4 on a scale of 0-10. Occasionally patients told us that a 7 on a 0-10 scale was an acceptable level or that anything above a 0 was not tolerable. This information is very valuable in elucidating areas that require some focused dialogue. Patient's perception of what is a tolerable level of pain may not match with the provider's perspective at all, but the mismatch will likely influence analgesic decision making on the part of both.

Non-Adherence

Thirty percent of patients were non-adherent to analgesic therapy regardless of whether or not they had a nurse dedicated to facilitating their pain management. This finding was very surprising to us. We knew, of course, that some patients did not want to take opioids, however, we were sure that when a “pain algorithm nurse” was involved in direct patient education and follow-up that they would be “compliant”. When asked about these findings, Betty Ferrell, PhD, RN would later relate to us that “patients have their own algorithms”. As a result we shifted from the term non-compliance to non-adherent, trying for a less judgemental approach. Patients make a conscious choice not to take their medications for a variety of reasons: side effects, a general feeling of being “anti-pill, fear or addiction, cost, a desire for natural remedies, a feeling of “being tough and fighting through” the pain and many others too numerous to mention.

We have come to use the term “negotiation” when discussing the relationship of medication adherence to ongoing treatment planning. The provider must ask the key question—“What of the medications ordered for you are you currently taking and how are you taking it?” A comparison of what has been ordered, what is actually being taken, and at least the degree of distress that any side effects are causing. For an example see the pain and side effect assessment tool in the Tools section of this manual. Analgesic decision making that is based on a combination of evidence-based drug choices, and a treatment plan that the patient is willing to adhere to will clearly produce the best outcomes.

Patient Satisfaction

Striving to be Patient-Centric

If nothing else health care in the 90's has pushed us more towards a patient-centric model. Patients have had to take more responsibility for figuring out their health coverage, and the advent of more preventive health care information in heart disease and oncology has pushed us towards more of a self-care model. Now with the internet providing virtually limitless health related information to the consumer it is not uncommon to find some patients that know more about the latest pain medicines than we do. The new buzz word is “patient participation”. For us this move toward patient-centric care is manifested in the patient self-report assessment that literally drives all aspects of the algorithm. In a sense, the patient directs their care via reporting the level of pain present initially, we validate their input by acting upon it, and subsequently they evulate that care by assessing the degree to which efficacy has been achieved. Over time is actually streamlines the process of patient care . . . and after all it is the patient's pain. We think you will find that moving more toward a patient-centric model will begin a rapid shift toward outcomes focused pain management.

Outcome Monitoring

Patient satisfaction has always been a part of the JCAHO regulations. With the new pain standards coming on line. pain management will need to become more routinely included in those satisfaction surveys. We asked patients to rate their satisfaction with pain management and generally patients were very satisfied, even in the presence of significant unrelieved pain. This is not without precedent in other pain management surveys. It is clear that patients feel that when surveyed about their care that they are been asked to participate in a referendum on their doctor . . . and for most oncology patients their doctor is saving their life. In the future we need to design patient satisfaction surveys that are specifically targeting their pain therapy, for example:

The effectiveness of the drug selected

The side effects of the drug selected

The dosing schedule of the drug selected

The cost of the drug selected

The availability of non-drug options

The more specifically we design these satisfaction surveys the less likely we will get answers that are not targeted enough to help us improve the system.

Follow-Up

If there is any one high risk area for failure it is surely in the area of follow-up. In the algorithm study in was not uncommon for patients to have had severe unrelieved pain for weeks to months. The algorithm provides specific reassessment parameters that clearly can be a challenge to meet in the current hectic environment, and don't always get achieved. For example in the setting of severe pain—defined as constant pain over a 7 on the pain scale—contact with the outpatient should be daily. However in many situations it took the nurse up to 7 days to make a follow-up phone call to the patient because of schedules and priorities. This sounds like an awfully long time for someone to have severe pain, however this is clearly an improvement over several weeks or months. In addition the referral to home care is indicated for patients with complex pain issues (of course reimbursement is now a major issue with home care referrals as well). The rigidity of the reassessment parameters is there to raise awareness of the need to continuously reassess and to set benchmarks for the system, in reality the clinician has the autonomy to flex the reassessment schedule based on the priorities of multiple patients all impinging at once.

Frequency of Re-Assessment

The highest level of re-assessment is extended to those patients with severe constant pain, particularly those patient who have had severe constant pain for many hours or days. Contact with those patients should be at least daily until the pain is at least under a 7 on the 0-10 scale. Generally patients have at least weekly contact for moderate pain (4-6 on the 0-10 scale) and at least weekly contact for significant side effects. The reader is referred to the various flow charts for specific reassessment frequent parameters. The frequency of reassessment is tied to pain intensity and side effects for the purpose of promoting rapid and intensive intervention during times where pain and side effects are uncontrolled. Not only does this promote better quality of life for our patients but if symptoms are rapidly brought back under control unnecessary hospitalizations may be avoided.

Method of Re-Assessment

In general patients with persistent severe pain and multiple severe side effects should be re-assessed urgently, probably in the hospital or at least in a extended outpatient visit. This allows for rapid opioid escalation in addition to any exam and diagnostics functions that the provider may require to plan further care. Early identification of the need for a home care nurse should be encouraged for patients with complex pain and side effect profiles, limited caregiver availability, and for patients who are generally too debilitated to come into the clinic frequently.

[image]

### Things to Report to Your Doctor or Nurse

[symbol] Any new pain

[symbol] Pain that is constantly above a 5 on a 0-10 scale even with your pain medicine

[symbol] Severe episodes of pain even with your pain medicine

[symbol] Stools that are hard and difficult to pass, or if you are moving your bowels only every 2^{nd} or 3^{rd} day or less

[symbol] Feeling very drowsy after taking your medicine

[symbol] Having bad dreams or “seeing things”

[symbol] Nausea, vomiting, or stomachache after taking your medicine

[symbol] Having dry mouth despite drinking fluids

[symbol] Muscle twitching or jerking

### Other Issues Important to Discuss with the Doctor or Nurse

[symbol] Not having enough instruction on how to take your medicines

[symbol] Not being able to afford your medicine

[symbol] Worries about taking pain medicine

[image]

[image]

[image]

[image]

A New Users Guide to WebX

WebX, the web-based algorithm reference tool, is a powerful mechanism for nurses, doctors, and clinicians that work with cancer patients. WebX allows the user to have access, twenty-four hours a day, to information about procedures, dosing guidelines, drug conversion tables, side effect protocols, sequential opioid trials, and up-to-date information relating to the treatment of cancer pain. The WebX tool is easy to use, is accessable from any computer with web accessibility, and provides a wealth of crucial information for the user with only a few keystrokes.

Going On-Line

Any computer that can access the World Wide Web can access the WebX program. Just like a person would go to a webpage about baseball or the daily news, you can “surf the web” to the WebX site just like you would to any other page. You don't need to learn any programming languages or have the help of your local computer guru to use our service, as it is designed to be intuitive even to users who may just be getting used to using the Web.

Getting to WebX On-Line

Get onto the Web using your computer's net-surfing software. Click on the address line with your mouse, and then delete everything that is already in the Address line. Once your address line is blank (much like a blank envelope that is yet to be addressed) you will want to type the following “web address” in using all lowercase letters.

www.painconsult.com

Once you have finished typing www.painconsult.com in on the Address line, hit the Enter key on the keyboard. In a few seconds, your computer should have “jumped” to the PainConsult.com page. which should have a title banner just like this:

[image]

[image]

[image]

Now, notice the brown and red subject headers on the right hand side. The larger brown headers represent categories of information, while the red headers are the specific tools, documents, and references within each section. (Imagine the brown headers being like a box, with the different tools, documents, and references being inside the box.) When you move your mouse over one of the red headers, it will turn blue, just like the menu options turned yellow when the mouse was over them. If you click on a blue subject header, you will be taken to the page it represents, much like flipping to an article in a magazine after looking up the page number in the table of contents.

Special Options

If you want to Search for a specific topic or title, you can click on the Search subject header to be taken to a screen where you can search for documents inside WebX. If there are certain pages that you want to have accessible all the time when you are using WebX, you will want to check out the Customize option. If you want to look at through a table of contents for a list of every document title, and tool within WebX you can use the Site Map to find things quickly, or to explore for things you haven't seen before! Lastly, if you want to quit your session in WebX, you merely need to click on the Log Off subject header to log out of the WebX program.

1. A patient pain management method, comprising:
assessing patient history;
determining a drug treatment in response to assessing patient history; and
repeatedly reassessing patient pain and assessing side-effects experienced by the patient and adjusting the drug treatment repeatedly to minimize patient pain.

2. A patient pain management method, comprising:
assessing patient history, comprising assessing patient pain and analgesic history;
determining an analgesic drug choice in response to assessing patient history;
administering the analgesic drug choice; and
reassessing patient pain and assessing side-effects experienced by the patient, and adjusting the analgesic drug choice, repeatedly, to minimize patient pain.

3. A process for pain treatment, comprising:
assessing pain and analgesic treatment history;
administering a pain treatment regimen in accordance with and in response to the assessment of pain and analgesic treatment history;
continuously reassessing pain and side-effects of the pain treatment regimen and adjusting the pain treatment regimen to minimize pain and negative side effects.

4. A system for pain management, comprising:
pain assessment tools for assessing a patient's pain and treatment history;
treatment choice tools for determining a pain treatment protocol in response to the assessment of the patient's pain and treatment history;
pain reassessment tools for reassessing patient pain in response to the pain treatment protocol; and
side-effect assessment tools for assessing the side-effects experienced by the patient in response to the pain treatment protocol.
